SEQUENCE LISTING (138 sequences)

SEQ ID NO: 1 - protein, 9 aa, synthetic construct
  REGION 1..9: note = synthetic peptide
  SITE 1: note = MISC_FEATURE - X is D or G
  PEPTIDE 1..9
  SITE 2: note = MISC_FEATURE - X is I or G
  SITE 4: note = MISC_FEATURE - X is V or L
  SITE 5: note = MISC_FEATURE - X is V or A
XXSXXYGLR

SEQ ID NO: 2 - protein, 15 aa, synthetic construct
  REGION 1..15: note = synthetic peptide
  PEPTIDE 1..15
  SITE 3: note = MISC_FEATURE - X is T or V
  SITE 4: note = MISC_FEATURE - X is Y or P
  SITE 5: note = MISC_FEATURE - X is D or N
  SITE 7: note = MISC_FEATURE - X is D or G
  SITE 8: note = MISC_FEATURE - X is I or G
  SITE 10: note = MISC_FEATURE - X is V or L
  SITE 11: note = MISC_FEATURE - X is V or A
VDXXXGXXSXXYGLR

SEQ ID NO: 3 - protein, 15 aa, synthetic construct
  REGION 1..15: note = synthetic peptide
  PEPTIDE 1..15
  SITE 5: note = MISC_FEATURE - X is D or N
  SITE 7: note = MISC_FEATURE - X is D or G
  SITE 8: note = MISC_FEATURE - X is I or G
  SITE 10: note = MISC_FEATURE - X is V or L
  SITE 11: note = MISC_FEATURE - X is V or A
VDTYXGXXSXXYGLR

SEQ ID NO: 4 - protein, 15 aa, synthetic construct
  REGION 1..15: note = synthetic peptide
  PEPTIDE 1..15
  SITE 7: note = MISC_FEATURE - X is D or G
  SITE 8: note = MISC_FEATURE - X is I or G
  SITE 10: note = MISC_FEATURE - X is V or L
  SITE 11: note = MISC_FEATURE - X is V or A
VDTYDGXXSXXYGLR

SEQ ID NO: 5 - protein, 15 aa, synthetic construct
  REGION 1..15: note = synthetic peptide
  PEPTIDE 1..15
  SITE 7: note = MISC_FEATURE - X is D or G
  SITE 8: note = MISC_FEATURE - X is I or G
VDTYDGXXSVVYGLR

SEQ ID NO: 6 - protein, 15 aa, synthetic construct
  REGION 1..15: note = synthetic peptide
  PEPTIDE 1..15
VDTYDGGISVVYGLR

SEQ ID NO: 7 - protein, 15 aa, synthetic construct
  REGION 1..15: note = synthetic peptide
  PEPTIDE 1..15
VDTYDGDISVVYGLR

SEQ ID NO: 8 - protein, 14 aa, synthetic construct
  REGION 1..14: note = synthetic peptide
  PEPTIDE 1..14
DTYDGDISVVYGLR

SEQ ID NO: 9 - protein, 14 aa, synthetic construct
  REGION 1..14: note = synthetic peptide
  PEPTIDE 1..14
TYDGDISVVYGLRS

SEQ ID NO: 10 - protein, 13 aa, synthetic construct
  REGION 1..13: note = synthetic peptide
  PEPTIDE 1..13
TYDGDISVVYGLR

SEQ ID NO: 11 - protein, 13 aa, synthetic construct
  REGION 1..13: note = synthetic peptide
  PEPTIDE 1..13
YDGDISVVYGLRS

SEQ ID NO: 12 - protein, 12 aa, synthetic construct
  REGION 1..12: note = synthetic peptide
  PEPTIDE 1..12
YDGDISVVYGLR

SEQ ID NO: 13 - protein, 12 aa, synthetic construct
  REGION 1..12: note = synthetic peptide
  PEPTIDE 1..12
DGDISVVYGLRS

SEQ ID NO: 14 - protein, 11 aa, synthetic construct
  REGION 1..11: note = synthetic peptide
  PEPTIDE 1..11
DGDISVVYGLR

SEQ ID NO: 15 - protein, 11 aa, synthetic construct
  REGION 1..11: note = synthetic peptide
  PEPTIDE 1..11
GDISVVYGLRS

SEQ ID NO: 16 - protein, 10 aa, synthetic construct
  REGION 1..10: note = synthetic peptide
  PEPTIDE 1..10
GDISVVYGLR

SEQ ID NO: 17 - protein, 10 aa, synthetic construct
  REGION 1..10: note = synthetic peptide
  PEPTIDE 1..10
DISVVYGLRS

SEQ ID NO: 18 - protein, 9 aa, synthetic construct
  REGION 1..9: note = synthetic peptide
  PEPTIDE 1..9
DISVVYGLR

SEQ ID NO: 19 - protein, 15 aa, synthetic construct
  REGION 1..15: note = synthetic peptide
  PEPTIDE 1..15
VDVPNGDISLAYGLR

SEQ ID NO: 20 - protein, 15 aa, synthetic construct
  REGION 1..15: note = synthetic peptide
  PEPTIDE 1..15
DVPNGDISLAYGLRS

SEQ ID NO: 21 - protein, 14 aa, synthetic construct
  REGION 1..14: note = synthetic peptide
  PEPTIDE 1..14
DVPNGDISLAYGLR

SEQ ID NO: 22 - protein, 14 aa, synthetic construct
  REGION 1..14: note = synthetic peptide
  PEPTIDE 1..14
VPNGDISLAYGLRS

SEQ ID NO: 23 - protein, 13 aa, synthetic construct
  REGION 1..13: note = synthetic peptide
  PEPTIDE 1..13
VPNGDISLAYGLR

SEQ ID NO: 24 - protein, 13 aa, synthetic construct
  REGION 1..13: note = synthetic peptide
  PEPTIDE 1..13
PNGDISLAYGLRS

SEQ ID NO: 25 - protein, 12 aa, synthetic construct
  REGION 1..12: note = synthetic peptide
  PEPTIDE 1..12
PNGDISLAYGLR

SEQ ID NO: 26 - protein, 12 aa, synthetic construct
  REGION 1..12: note = synthetic peptide
  PEPTIDE 1..12
NGDISLAYGLRS

SEQ ID NO: 27 - protein, 11 aa, synthetic construct
  REGION 1..11: note = synthetic peptide
  PEPTIDE 1..11
NGDISLAYGLR

SEQ ID NO: 28 - protein, 11 aa, synthetic construct
  REGION 1..11: note = synthetic peptide
  PEPTIDE 1..11
GDISLAYGLRS

SEQ ID NO: 29 - protein, 10 aa, synthetic construct
  REGION 1..10: note = synthetic peptide
  PEPTIDE 1..10
GDISLAYGLR

SEQ ID NO: 30 - protein, 10 aa, synthetic construct
  REGION 1..10: note = synthetic peptide
  PEPTIDE 1..10
DISLAYGLRS

SEQ ID NO: 31 - protein, 9 aa, synthetic construct
  REGION 1..9: note = synthetic peptide
  PEPTIDE 1..9
DISLAYGLR

SEQ ID NO: 32 - protein, 15 aa, synthetic construct
  REGION 1..15: note = synthetic peptide
  PEPTIDE 1..15
VDTYDGDGSVVYGLR

SEQ ID NO: 33 - protein, 15 aa, synthetic construct
  REGION 1..15: note = synthetic peptide
  PEPTIDE 1..15
VDVPEGDISLAYGLR

SEQ ID NO: 34 - protein, 14 aa, synthetic construct
  REGION 1..14: note = synthetic peptide
  PEPTIDE 1..14
VDTYDGDISVVYGL

SEQ ID NO: 35 - protein, 13 aa, synthetic construct
  REGION 1..13: note = synthetic peptide
  PEPTIDE 1..13
VDTYDGDISVVYG

SEQ ID NO: 36 - protein, 12 aa, synthetic construct
  REGION 1..12: note = synthetic peptide
  PEPTIDE 1..12
VDTYDGDISVVY

SEQ ID NO: 37 - protein, 11 aa, synthetic construct
  REGION 1..11: note = synthetic peptide
  PEPTIDE 1..11
VDTYDGDISVV

SEQ ID NO: 38 - protein, 10 aa, synthetic construct
  REGION 1..10: note = synthetic peptide
  PEPTIDE 1..10
VDTYDGDISV

SEQ ID NO: 39 - protein, 9 aa, synthetic construct
  REGION 1..9: note = synthetic peptide
  PEPTIDE 1..9
VDTYDGDIS

SEQ ID NO: 40 - protein, 16 aa, synthetic construct
  REGION 1..16: note = synthetic peptide
  PEPTIDE 1..16
VDTYDGRGDSVVYGLR

SEQ ID NO: 41 - protein, 14 aa, synthetic construct
  REGION 1..14: note = synthetic peptide
  PEPTIDE 1..14
VDVPNGDISLAYGL

SEQ ID NO: 42 - protein, 13 aa, synthetic construct
  REGION 1..13: note = synthetic peptide
  PEPTIDE 1..13
VDVPNGDISLAYG

SEQ ID NO: 43 - protein, 11 aa, synthetic construct
  REGION 1..11: note = synthetic peptide
  PEPTIDE 1..11
VDVPNGDISLA

SEQ ID NO: 44 - protein, 9 aa, synthetic construct
  REGION 1..9: note = synthetic peptide
  PEPTIDE 1..9
VDVPNGDIS

SEQ ID NO: 45 - protein, 16 aa, synthetic construct
  REGION 1..16: note = synthetic peptide
  PEPTIDE 1..16
GDPNDGRGDSVVYGLR

SEQ ID NO: 46 - protein, 14 aa, synthetic construct
  REGION 1..14: note = synthetic peptide
  PEPTIDE 1..14
GDPNGDISVVYGLR

SEQ ID NO: 47 - protein, 15 aa, synthetic construct
  REGION 1..15: note = synthetic peptide
  PEPTIDE 1..15
VDVPNGDISLAYRLR

SEQ ID NO: 48 - protein, 15 aa, synthetic construct
  REGION 1..15: note = synthetic peptide
  PEPTIDE 1..15
VDVPEGDISLAYRLR

SEQ ID NO: 49 - protein, 15 aa, synthetic construct
  REGION 1..15: note = synthetic peptide
  PEPTIDE 1..15
  SITE 2: note = MISC_FEATURE - X is beta D
VXTYDGDISVVYGLR

SEQ ID NO: 50 - protein, 15 aa, synthetic construct
  REGION 1..15: note = synthetic peptide
  PEPTIDE 1..15
  SITE 5: note = MISC_FEATURE - X is beta D
VDTYXGDISVVYGLR

SEQ ID NO: 51 - protein, 15 aa, synthetic construct
  REGION 1..15: note = synthetic peptide
  SITE 7: note = MISC_FEATURE - X is beta D
VDTYDGXISVVYGLR

SEQ ID NO: 52 - protein, 15 aa, synthetic construct
  REGION 1..15: note = synthetic peptide
  PEPTIDE 1..15
VDTYDGDISVVYGLS

SEQ ID NO: 53 - protein, 13 aa, synthetic construct
  REGION 1..13: note = synthetic peptide
  PEPTIDE 1..13
DTYDGDISVVYGL

SEQ ID NO: 54 - protein, 12 aa, synthetic construct
  REGION 1..12: note = synthetic peptide
  PEPTIDE 1..12
DTYDGDISVVYG

SEQ ID NO: 55 - protein, 12 aa, synthetic construct
  REGION 1..12: note = synthetic peptide
  PEPTIDE 1..12
TYDGDISVVYGL

SEQ ID NO: 56 - protein, 11 aa, synthetic construct
  REGION 1..11: note = synthetic peptide
  PEPTIDE 1..11
DTYDGDISVVY

SEQ ID NO: 57 - protein, 11 aa, synthetic construct
  REGION 1..11: note = synthetic peptide
  PEPTIDE 1..11
TYDGDISVVYG

SEQ ID NO: 58 - protein, 11 aa, synthetic construct
  REGION 1..11: note = synthetic peptide
  PEPTIDE 1..11
YDGDISVVYGL

SEQ ID NO: 59 - protein, 10 aa, synthetic construct
  REGION 1..10: note = synthetic peptide
  PEPTIDE 1..10
DTYDGDISVV

SEQ ID NO: 60 - protein, 10 aa, synthetic construct
  REGION 1..10: note = synthetic peptide
  PEPTIDE 1..10
TYDGDISVVY

SEQ ID NO: 61 - protein, 10 aa, synthetic construct
  REGION 1..10: note = synthetic peptide
  PEPTIDE 1..10
YDGDISVVYG

SEQ ID NO: 62 - protein, 10 aa, synthetic construct
  REGION 1..10: note = synthetic peptide
  PEPTIDE 1..10
DGDISVVYGL

SEQ ID NO: 63 - protein, 9 aa, synthetic construct
  REGION 1..9: note = synthetic peptide
  PEPTIDE 1..9
VDTYDGDIS

SEQ ID NO: 64 - protein, 9 aa, synthetic construct
  REGION 1..9: note = synthetic peptide
  PEPTIDE 1..9
DTYDGDISV

SEQ ID NO: 65 - protein, 9 aa, synthetic construct
  REGION 1..9: note = synthetic peptide
  PEPTIDE 1..9
TYDGDISVV

SEQ ID NO: 66 - protein, 9 aa, synthetic construct
  REGION 1..9: note = synthetic peptide
  PEPTIDE 1..9
YDGDISVVY

SEQ ID NO: 67 - protein, 9 aa, synthetic construct
  REGION 1..9: note = synthetic peptide
  PEPTIDE 1..9
DGDISVVYG

SEQ ID NO: 68 - protein, 9 aa, synthetic construct
  REGION 1..9: note = synthetic peptide
  PEPTIDE 1..9
GDISVVYGL

SEQ ID NO: 69 - protein, 9 aa, synthetic construct
  REGION 1..9: note = synthetic peptide
  PEPTIDE 1..9
ISVVYGLRS

SEQ ID NO: 70 - protein, 8 aa, synthetic construct
  REGION 1..8: note = synthetic peptide
  PEPTIDE 1..8
VDTYDGDI

SEQ ID NO: 71 - protein, 8 aa, synthetic construct
  REGION 1..8: note = synthetic peptide
  PEPTIDE 1..8
DTYDGDIS

SEQ ID NO: 72 - protein, 8 aa, synthetic construct
  REGION 1..8: note = synthetic peptide
  PEPTIDE 1..8
TYDGDISV

SEQ ID NO: 73 - protein, 8 aa, synthetic construct
  REGION 1..8: note = synthetic peptide
  PEPTIDE 1..8
YDGDISVV

SEQ ID NO: 74 - protein, 8 aa, synthetic construct
  REGION 1..8: note = synthetic peptide
  PEPTIDE 1..8
DGDISVVY

SEQ ID NO: 75 - protein, 8 aa, synthetic construct
  REGION 1..8: note = synthetic peptide
  PEPTIDE 1..8
GDISVVYG

SEQ ID NO: 76 - protein, 8 aa, synthetic construct
  REGION 1..8: note = synthetic peptide
  PEPTIDE 1..8
DISVVYGL

SEQ ID NO: 77 - protein, 8 aa, synthetic construct
  REGION 1..8: note = synthetic peptide
  PEPTIDE 1..8
ISVVYGLR

SEQ ID NO: 78 - protein, 13 aa, synthetic construct
  REGION 1..13: note = synthetic peptide
  PEPTIDE 1..13
DVPNGDISLAYGL

SEQ ID NO: 79 - protein, 12 aa, synthetic construct
  REGION 1..12: note = synthetic peptide
  PEPTIDE 1..12
VDVPNGDISLAY

SEQ ID NO: 80 - protein, 12 aa, synthetic construct
  REGION 1..12: note = synthetic peptide
  PEPTIDE 1..12
DVPNGDISLAYG

SEQ ID NO: 81 - protein, 12 aa, synthetic construct
  REGION 1..12: note = synthetic peptide
  PEPTIDE 1..12
VPNGDISLAYGL

SEQ ID NO: 82 - protein, 11 aa, synthetic construct
  REGION 1..11: note = synthetic peptide
  PEPTIDE 1..11
DVPNGDISLAY

SEQ ID NO: 83 - protein, 11 aa, synthetic construct
  REGION 1..11: note = synthetic peptide
  PEPTIDE 1..11
VPNGDISLAYG

SEQ ID NO: 84 - protein, 11 aa, synthetic construct
  REGION 1..11: note = synthetic peptide
  PEPTIDE 1..11
PNGDISLAYGL

SEQ ID NO: 85 - protein, 10 aa, synthetic construct
  REGION 1..10: note = synthetic peptide
  PEPTIDE 1..10
VDVPNGDISL

SEQ ID NO: 86 - protein, 10 aa, synthetic construct
  REGION 1..10: note = synthetic peptide
  PEPTIDE 1..10
DVPNGDISLA

SEQ ID NO: 87 - protein, 10 aa, synthetic construct
  REGION 1..10: note = synthetic peptide
  PEPTIDE 1..10
VPNGDISLAY

SEQ ID NO: 88 - protein, 10 aa, synthetic construct
  REGION 1..10: note = synthetic peptide
  PEPTIDE 1..10
PNGDISLAYG

SEQ ID NO: 89 - protein, 10 aa, synthetic construct
  REGION 1..10: note = synthetic peptide
  PEPTIDE 1..10
NGDISLAYGL

SEQ ID NO: 90 - protein, 9 aa, synthetic construct
  REGION 1..9: note = synthetic peptide
  PEPTIDE 1..9
DVPNGDISL

SEQ ID NO: 91 - protein, 9 aa, synthetic construct
  REGION 1..9: note = synthetic peptide
  PEPTIDE 1..9
VPNGDISLA

SEQ ID NO: 92 - protein, 9 aa, synthetic construct
  REGION 1..9: note = synthetic peptide
  PEPTIDE 1..9
PNGDISLAY

SEQ ID NO: 93 - protein, 9 aa, synthetic construct
  REGION 1..9: note = synthetic peptide
  PEPTIDE 1..9
NGDISLAYG

SEQ ID NO: 94 - protein, 9 aa, synthetic construct
  REGION 1..9: note = synthetic peptide
  PEPTIDE 1..9
GDISLAYGL

SEQ ID NO: 95 - protein, 9 aa, synthetic construct
  REGION 1..9: note = synthetic peptide
  PEPTIDE 1..9
ISLAYGLRS

SEQ ID NO: 96 - protein, 8 aa, synthetic construct
  REGION 1..8: note = synthetic peptide
  PEPTIDE 1..8
VDVPNGDI

SEQ ID NO: 97 - protein, 8 aa, synthetic construct
  REGION 1..8: note = synthetic peptide
  PEPTIDE 1..8
DVPNGDIS

SEQ ID NO: 98 - protein, 8 aa, synthetic construct
  REGION 1..8: note = synthetic peptide
  PEPTIDE 1..8
VPNGDISL

SEQ ID NO: 99 - protein, 8 aa, synthetic construct
  REGION 1..8: note = synthetic peptide
  PEPTIDE 1..8
PNGDISLA

SEQ ID NO: 100 - protein, 8 aa, synthetic construct
  REGION 1..8: note = synthetic peptide
  PEPTIDE 1..8
NGDISLAY

SEQ ID NO: 101 - protein, 8 aa, synthetic construct
  REGION 1..8: note = synthetic peptide
  PEPTIDE 1..8
GDISLAYG

SEQ ID NO: 102 - protein, 8 aa, synthetic construct
  REGION 1..8: note = synthetic peptide
  PEPTIDE 1..8
DISLAYGL

SEQ ID NO: 103 - protein, 8 aa, synthetic construct
  REGION 1..8: note = synthetic peptide
  PEPTIDE 1..8
ISLAYGLR

SEQ ID NO: 104 - protein, 16 aa, synthetic construct
  REGION 1..16: note = synthetic peptide
  PEPTIDE 1..16
VDVPNGRGDSLAYGLR

SEQ ID NO: 105 - protein, 7 aa, synthetic construct
  REGION 1..7: note = synthetic peptide
  SITE 1: note = MISC_FEATURE - X is E or G
  PEPTIDE 1..7
  SITE 3: note = MISC_FEATURE - X is S or T
XLXYGIK

SEQ ID NO: 106 - protein, 16 aa, synthetic construct
  REGION 1..16: note = synthetic peptide
  PEPTIDE 1..16
  SITE 2: note = MISC_FEATURE - X is C, P or G
  SITE 5: note = MISC_FEATURE - X is E or G
  SITE 6: note = MISC_FEATURE - X is C, Dor I
  SITE 7: note = MISC_FEATURE - X is D, I, S or G
  SITE 8: note = MISC_FEATURE - X is S, D or G
  SITE 10: note = MISC_FEATURE - X is E or G
  SITE 12: note = MISC_FEATURE - X is S or T
KXLAXXXXIXLXYGIK

SEQ ID NO: 107 - protein, 16 aa, synthetic construct
  REGION 1..16: note = synthetic peptide
  PEPTIDE 1..16
  SITE 2: note = MISC_FEATURE - X is C, P or G
  SITE 5: note = MISC_FEATURE - X is E or G
  SITE 6: note = MISC_FEATURE - X is C, I or absent
  SITE 7: note = MISC_FEATURE - X is D, G or absent
  SITE 8: note = MISC_FEATURE - X is S, G or absent
  SITE 10: note = MISC_FEATURE - X is E or G
KXLAXXXXIXLSYGIK

SEQ ID NO: 108 - protein, 13 aa, synthetic construct
  REGION 1..13: note = synthetic peptide
  PEPTIDE 1..13
  SITE 2: note = MISC_FEATURE - X is C, P or G
  SITE 5: note = MISC_FEATURE - X is E or G
  SITE 7: note = MISC_FEATURE - X is E or G
KXLAXIXLSYGIK

SEQ ID NO: 109 - protein, 8 aa, synthetic construct
  REGION 1..8: note = synthetic peptide
  PEPTIDE 1..8
IELSYGIK

SEQ ID NO: 110 - protein, 13 aa, synthetic construct
  REGION 1..13: note = synthetic peptide
  PEPTIDE 1..13
AEIDSIELSYGIK

SEQ ID NO: 111 - protein, 16 aa, synthetic construct
  REGION 1..16: note = synthetic peptide
  PEPTIDE 1..16
KPLAEIDSIELSYGIK

SEQ ID NO: 112 - protein, 16 aa, synthetic construct
  REGION 1..16: note = synthetic peptide
  REGION 1..16: note = MISC_FEATURE - cyclic peptide
KCLAECDSIELSYGIK

SEQ ID NO: 113 - protein, 16 aa, synthetic construct
  REGION 1..16: note = synthetic peptide
  PEPTIDE 1..16
KPLAEDISIELSYGIK

SEQ ID NO: 114 - protein, 16 aa, synthetic construct
  REGION 1..16: note = synthetic peptide
  PEPTIDE 1..16
KPLAEISDIELSYGIK

SEQ ID NO: 115 - protein, 16 aa, synthetic construct
  REGION 1..16: note = synthetic peptide
  PEPTIDE 1..16
KPLAEIGDIELSYGIK

SEQ ID NO: 116 - protein, 15 aa, synthetic construct
  REGION 1..15: note = synthetic peptide
  PEPTIDE 1..15
KPLAEGDIELSYGIK

SEQ ID NO: 117 - protein, 13 aa, synthetic construct
  REGION 1..13: note = synthetic peptide
  PEPTIDE 1..13
KPLAEIELSYGIK

SEQ ID NO: 118 - protein, 16 aa, synthetic construct
  REGION 1..16: note = synthetic peptide
  PEPTIDE 1..16
KPLAEIDSIELTYGIK

SEQ ID NO: 119 - protein, 16 aa, synthetic construct
  REGION 1..16: note = synthetic peptide
  PEPTIDE 1..16
KPLAEIDGIELSYGIK

SEQ ID NO: 120 - protein, 16 aa, synthetic construct
  REGION 1..16: note = synthetic peptide
  PEPTIDE 1..16
KPLAEIDGIELTYGIK

SEQ ID NO: 121 - protein, 16 aa, synthetic construct
  REGION 1..16: note = synthetic peptide
  PEPTIDE 1..16
KPLAEIGSIELSYGIK

SEQ ID NO: 122 - protein, 16 aa, synthetic construct
  REGION 1..16: note = synthetic peptide
  PEPTIDE 1..16
KGLAEIDSIELSYGIK

SEQ ID NO: 123 - protein, 16 aa, synthetic construct
  REGION 1..16: note = synthetic peptide
  PEPTIDE 1..16
KPLAGIDSIGLSYGIK

SEQ ID NO: 124 - protein, 16 aa, synthetic construct
  REGION 1..16: note = synthetic peptide
  REGION 1..16: note = misc_feature - cyclic peptide
KCLAEIDSCELSYGIK

SEQ ID NO: 125 - protein, 14 aa, synthetic construct
  REGION 1..14: note = synthetic peptide
  PEPTIDE 1..14
LAEIDSIELSYGIK

SEQ ID NO: 126 - protein, 12 aa, synthetic construct
  REGION 1..12: note = synthetic peptide
  PEPTIDE 1..12
EIDSIELSYGIK

SEQ ID NO: 127 - protein, 11 aa, synthetic construct
  REGION 1..11: note = synthetic peptide
  PEPTIDE 1..11
IDSIELSYGIK

SEQ ID NO: 128 - protein, 10 aa, synthetic construct
  REGION 1..10: note = synthetic peptide
  PEPTIDE 1..10
DSIELSYGIK

SEQ ID NO: 129 - protein, 9 aa, synthetic construct
  REGION 1..9: note = synthetic peptide
  PEPTIDE 1..9
SIELSYGIK

SEQ ID NO: 130 - protein, 8 aa, synthetic construct
  REGION 1..8: note = synthetic peptide
  REGION 1..8: note = MISC_FEATURE - cyclic peptide
CLAEIDSC

SEQ ID NO: 131 - protein, 18 aa, synthetic construct
  REGION 1..18: note = synthetic peptide
  REGION 1..18: note = MISC_FEATURE - cyclic peptide
CFKPLAEIDSIECSYGIK

SEQ ID NO: 132 - protein, 13 aa, synthetic construct
  REGION 1..13: note = synthetic peptide
  REGION 1..13: note = MISC_FEATURE - cyclic peptide
CFKPLAEIDSIEC

SEQ ID NO: 133 - protein, 15 aa, synthetic construct
  REGION 1..15: note = synthetic peptide
  PEPTIDE 1..15
KPLAEIDSIELSYGI

SEQ ID NO: 134 - protein, 14 aa, synthetic construct
  REGION 1..14: note = synthetic peptide
  PEPTIDE 1..14
KPLAEIDSIELSYG

SEQ ID NO: 135 - protein, 13 aa, synthetic construct
  REGION 1..13: note = synthetic peptide
  PEPTIDE 1..13
KPLAEIDSIELSY

SEQ ID NO: 136 - protein, 12 aa, synthetic construct
  REGION 1..12: note = synthetic peptide
  PEPTIDE 1..12
KPLAEIDSIELS

SEQ ID NO: 137 - protein, 11 aa, synthetic construct
  REGION 1..11: note = synthetic peptide
  PEPTIDE 1..11
KPLAEIDSIEL

SEQ ID NO: 138 - protein, 10 aa, synthetic construct
  REGION 1..10: note = synthetic peptide
  PEPTIDE 1..10
KPLAEIDSIE

(19) United States
(12) Patent Application Publication
(10) Pub. No.: US 2025/0326799 A1
(43) Pub. Date: Oct. 23, 2025

(54) AGENTS INDUCING VASCULARISATION

(71) Applicant: Coegin Pharma AB, Lund (SE)

(72) Inventors: Anna Hultgårdh Nilsson, Genarp (SE); Jan Nilsson, Genarp (SE)

(21) Appl. No.: 18/864,609

(22) PCT Filed: May 15, 2023
(86) PCT No.: PCT/EP2023/062956

§ 371 Date: Nov. 11, 2024

(30) Foreign Application Priority Data
May 13, 2022 (EP) 22173230.8

(51) Int. Cl. C07K 7/64; A61K 38/00; A61L 31/10; C07K 7/06; C07K 7/08; C08L 89/00; C12N 9/10

(52) U.S. Cl. CPC C07K 7/64; A61L 31/10; C07K 7/06; C07K 7/08; C08L 89/00; C12N 9/1088; A61K 38/00; C07K 2319/21; C07K 2319/705; C12Y 205/01018

(57) ABSTRACT

The present invention relates to agents capable of inducing vascularisation. The disclosure also relates to treatment of diseases, such as cardiovascular diseases using said agents.

### TECHNICAL FIELD

The present invention relates to agents capable of inducing vascularisation. The disclosure also relates to treatment of diseases, such as cardiovascular diseases using said agents.

### BACKGROUND

Cardiovascular disease (CVD) is a class of diseases that involve the heart or blood vessels. CVD includes coronary artery diseases (CAD) such as angina and myocardial infarction (commonly known as a heart attack). Other CVDs include stroke, heart failure, hypertensive heart disease, rheumatic heart disease, cardiomyopathy, abnormal heart rhythms, congenital heart disease, valvular heart disease, carditis, aortic aneurysms, peripheral artery disease, thromboembolic disease, and venous thrombosis.

The underlying mechanisms vary depending on the disease. It is estimated that dietary risk factors are associated with 53% of CVD deaths. Coronary artery disease, stroke, and peripheral artery disease involve atherosclerosis. This may be caused by high blood pressure, smoking, diabetes mellitus, lack of exercise, obesity, high blood cholesterol, poor diet, excessive alcohol consumption, and poor sleep, among other things. High blood pressure is estimated to account for approximately 13% of CVD deaths, while tobacco accounts for 9%, diabetes 6%, lack of exercise 6%, and obesity 5%. Rheumatic heart disease may follow untreated strep throat.

### SUMMARY

The inventors of the present disclosure have identified peptides capable of inducing vascularisation. Thus, said peptides are promising candidates for the treatment of various diseases or disorders associated with reduced angiogenesis, including cardiovascular diseases.

One aspect of the disclosure provides for an agent comprising:
a) a peptide selected from the group consisting of:
(i) a peptide comprising or consisting of an amino acid sequence of the general formula:

(SEQ ID NO: 1)
X₅X₆SX₇X₈YGLR

wherein:
X₅ is D or G;
X₆ is I or G;
X₇ is V or L;
X₈ is V or A;
(ii) a peptide comprising or consisting of an amino acid sequence of the general formula:

(SEQ ID NO: 105)
X₁₄LX₁₅YGIK

wherein:
X₁₄ is E or G;
X₁₅ is S or T;
(iii) a peptide comprising or consisting of an amino acid sequence selected from the group consisting of:

(SEQ ID NO: 34)
VDTYDGDISVVYGL,
(SEQ ID NO: 35)
VDTYDGDISVVYG,
(SEQ ID NO: 36)
VDTYDGDISVVY,
(SEQ ID NO: 37)
VDTYDGDISVV,
(SEQ ID NO: 38)
VDTYDGDISV,
(SEQ ID NO: 39)
VDTYDGDIS,
(SEQ ID NO: 40)
VDTYDGRGDSVVYGLR,
(SEQ ID NO: 41)
VDVPNGDISLAYGL,
(SEQ ID NO: 42)
VDVPNGDISLAYG,
(SEQ ID NO: 43)
VDVPNGDISLA,
(SEQ ID NO: 44)
VDVPNGDIS,
(SEQ ID NO: 45)
GDPNDGRGDSVVYGLR,
(SEQ ID NO: 46)
LDGLVRAYDNISPVG,
(SEQ ID NO: 47)
GDPNGDISVVYGLR
(SEQ ID NO: 48)
VDVPNGDISLAYRLR,
(SEQ ID NO: 49)
VDVPEGDISLAYRLR,
(SEQ ID NO: 50)
V(beta-D)TYDGDISVVYGLR,
(SEQ ID NO: 51)
VDTY(beta-D)GDISVVYGLR,
(SEQ ID NO: 52)
VDTYDG(beta-D)ISVVYGLR;
(SEQ ID NO: 130)
CLAEIDSC (Cyclic),
(SEQ ID NO: 131)
CFKPLAEIDSIECSYGIK (Cyclic),
(SEQ ID NO: 132)
Cyclic CFKPLAEIDSIEC,
(SEQ ID NO: 133)
KPLAEIDSIELSYGI,
(SEQ ID NO: 134)
KPLAEIDSIELSYG,
(SEQ ID NO: 135)
KPLAEIDSIELSY,
(SEQ ID NO: 136)
KPLAEIDSIELS,
(SEQ ID NO: 137)
KPLAEIDSIEL,
and
(SEQ ID NO: 138)
KPLAEIDSIE;

b) a polynucleotide encoding upon expression, the peptide of a);
c) a vector comprising the polynucleotide of b); or
d) a cell comprising the polynucleotide of b), or the vector of c),
   for use in the treatment of and/or the prevention of a disease or disorder associated with reduced or impaired angiogenesis in a subject.

In one aspect, the present invention relates to a method for treating or preventing a disease or disorder in a subject, wherein the disease or disorder is selected from the group consisting of:
i. a disease of the circulatory system,
ii. an injury of external cause,
iii. a disease of the immune system,
iv. a disease of the nervous system, and
v. a disease of the musculoskeletal system or connective tissue,
   the method comprising administering a therapeutically effective amount of an agent as herein to an subject in need thereof.

In one aspect, the present invention relates to use of an agent as defined herein for the manufacture of a medicament for the treatment or prevention of a disease or disorder selected from the group consisting of:
i. a disease of the circulatory system,
ii. an injury of external cause,
iii. a disease of the immune system,
iv. a disease of the nervous system, and
v. a disease of the musculoskeletal system or connective tissue.

In one aspect, the present invention relates to an agent as defined herein for use in improving vascularisation post surgery in a subject.

In one aspect, the present invention relates to a method for inducing vascularization, said method comprising administering the agent as defined herein to a subject.

In one aspect, the present invention relates to an implant comprising a peptide as defined herein.

### DESCRIPTION OF DRAWINGS

FIG. 1: FOL26 stimulates endothelial and vascular smooth muscle cell proliferation. Human umbilical cord endothelial cells (A) and human coronary artery smooth muscle cells (B) were treated with FOL26 in concentrations of 10 nM, 100 nM, and 1000 nM, and DNA synthesis analyzed by measuring the uptake of BrdU. An increase in O.D. (450 nm) for the FOL 26 treated cell lines compared to the control.

FIG. 2: FOL26 enhances endothelial cell tube formation and expression of PECAM-1. Human umbilical cord endothelial cells were treated with FOL26 in concentrations of 10 nM, 100 nM, and 1000 nM, and endothelial tube formation and gene expression of PECAM-1 (CD31) analyzed. FOL26 increased tube formation compared to control. The expression of PECAM-1 was increased for 100 nM and 1000 nM FOL26.

FIG. 3: FOL26 inhibits endothelial and arterial smooth muscle cell apoptosis. Cultured human umbilical cord endothelial and coronary artery smooth cells were exposed to soluble Fas ligand (sFasL) to induce apoptosis that subsequently was assessed by determining activation of caspase 3 and 7. Cells were also treated with FOL 26 in concentrations of 10 nM, 100 nM, and 1000 nM to determine the effect on apoptosis activated by sFasL. FOL26 was capable of reversing the sFasL-induced apoptosis, to the highest extent at the 100 nM concentration.

FIG. 4: FOL26 affects the gene expression of VEGF and the VEGF receptor family in endothelial cells. Human umbilical cord endothelial cells were treated with FOL 26 in concentrations of 10 nM, 100 nM, and 1000 nM and the expression of the genes encoding c-Met, neuropilin-1 (NRP-1), VEGF-A and VEGF receptor-2 (VEGFR-2) determined by polymerase chain reaction (PCR). FOL026 was reducing the c-Met expression, in the lowest dose reducing however in the 100 nM and 1000 nM doses were inducing NRP-1 expression. FOL026 had no effect on VEGF-A expression, however in 10 nM it reduced and in 1000 nM it induced the expression of VEGFR-2 levels.

FIG. 5: FOL26 affects the gene expression of cytokines and matrix metalloproteinases in endothelial cells. Human umbilical cord endothelial cells were treated with FOL 26 in concentrations of 10 nM, 100 nM, and 1000 nM and the expression of the genes encoding TNF-α, IL-6, MMP2 and MMP3 determined by polymerase chain reaction (PCR). TNF-α is strong pro-inflammatory cytokine while MMP2 is required for activation of smooth muscle cell proliferation and migration. FOL026 reduced the TNF-α expression in 10 nM and 100 nM doses, had no effect on IL-6 or MMP3 expression, however, MMP2 expression was induced with 200 nM and 1000 nM doses.

FIG. 6: FOL26 affects the gene expression of VEGF and the VEGF receptor family in arterial smooth muscle cells. Human coronary artery smooth cells were treated with FOL26 in concentrations of 10 nM, 100 nM, and 1000 nM and the expression of the genes encoding c-Met, neuropilin-1 (NRP-1), VEGF-A and VEGF receptor-2 (VEGFR-2) determined by polymerase chain reaction (PCR). FOL026 reduced the c-MET expression in 10 nM and 1000 nM doses, as well as the NRP-1 expression in 10, 100, and 1000 nM doses. FOL026 induced in 10, 100, and 1000 nM doses the VEGF-A expression, as well as for FOL026 100 nM dose only.

FIG. 7: FOL26 affects the gene expression of cytokines and matrix metalloproteinases in arterial smooth muscle cells. Human coronary artery smooth cells were treated with FOL 26 in concentrations of 10 nM, 100 nM, and 1000 nM and the expression of the genes encoding TNF-α, IL-6, MMP2 and MMP3 determined by polymerase chain reaction (PCR). TNF-α is strong pro-inflammatory cytokine while MMP2 is required for activation of smooth muscle cell proliferation and migration. FOL026 100 and 1000 nM induced the TNF-α, IL-6 and MMP3 expression, furthermore, FOL026 induced in 10, 100, and 1000 nM doses the MMP2 expression.

FIG. 8: FOL26 stimulation of endothelial cell proliferation is NRP-1 dependent. Human umbilical cord endothelial cells were first treated with a scramble non-coding siRNA (NC) or siRNA for NRP-1, subsequently they were treated with FOL26 in concentrations of 10 nM, 100 nM, and 1000 nM, and DNA synthesis analyzed by measuring the uptake of BrdU. An increase in O.D. (450 nm) for the FOL 26 treated cell lines compared to the control showed that FOL26 induced angiogenesis may be NRP-1 dependent as a reduction was observed for FOL26 10 and 100 nM.

FIG. 9: Human umbilical cord endothelial cells were treated with a scramble non-coding siRNA (NC) or siRNA for NRP-1 in concentrations of 1 nM, and 5 nM, and the NRP-1 expression values and Glyceraldehyde-phosphate dehydrogenase (GAPDH) mRNA expression values was evaluated, the latter was used as an internal control to normalize the data.

FIG. 10: FOL26 enhances endothelial cell tube in an NRP-1 specific manner. Human umbilical cord endothelial cells were first treated with a scramble non-coding siRNA (NC) or siRNA for NRP-1, subsequently treated with FOL26 in concentrations of 10 nM, 100 nM, and 1000 nM, and endothelial tube formation and the total master segemnts were measured using ImageJ. gene expression of PECAM-1 (CD31) analyzed. FOL26 increased tube formation compared to control in a NRP-1 dependent way. Scale bar: 150 μm.

FIG. 11: FOL56 stimulates endothelial and vascular smooth muscle cell proliferation. Human umbilical cord endothelial cells (A) and human coronary artery smooth muscle cells (B) were treated with FOL 56 in concentrations of 10 nM, 100 nM, and 1000 nM and DNA synthesis analyzed by measuring the uptake of BrdU. FOL56 increases proliferation at all concentrations in both HUVECs and HCASMCs compared to the control.

FIG. 12: FOL56 enhances endothelial cell tube formation and expression of PECAM-1. Human umbilical cord endothelial cells were treated with FOL 56 in concentrations of 10 nM, 100 nM, and 1000 nM and endothelial tube formation and gene expression of PECAM-1 (CD31) analyzed. FOL56 increased tube formation at all concentrations compared to control, and also increased expression of PECAM-1.

FIG. 13: FOL56 inhibits endothelial and arterial smooth muscle cell apoptosis. Cultured human umbilical cord endothelial and coronary artery smooth cells were exposed to soluble Fas ligand (sFasL) to induce apoptosis that subsequently was assessed by determining activation of caspase 3 and 7. Cells were also treated with FOL 56 in concentrations of 10 nM, 100 nM, and 1000 nM to determine the effect on apoptosis activated by sFasL. FOL56 was capable of inhibiting, or trended towards inhibiting apoptosis in both HUVECs and HCASMCs.

FIG. 14: FOL56 affects the gene expression of VEGF and the VEGF receptor family in endothelial cells. Human umbilical cord endothelial cells were treated with FOL 56 in concentrations of 10 nM, 100 nM, and 1000 nM and the expression of the genes encoding c-Met, neuropilin-1 (NRP-1), VEGF-A and VEGF receptor-2 (VEGFR-2) determined by polymerase chain reaction (PCR).

FIG. 15: FOL56 affects the gene expression of cytokines and matrix metalloproteinases in endothelial cells. Human umbilical cord endothelial cells were treated with FOL 56 in concentrations of 10 nM, 100 nM, and 1000 nM and the expression of the genes encoding TNF-α, IL-6, MMP2 and MMP3 determined by polymerase chain reaction (PCR). TNF-α is strong pro-inflammatory cytokine while MMP2 is required for activation of smooth muscle cell proliferation and migration.

FIG. 16: FOL56 affects the gene expression of VEGF and the VEGF receptor family in arterial smooth muscle cells. Human coronary artery smooth cells were treated with FOL 56 in concentrations of 10 nM, 100 nM, and 1000 nM and the expression of the genes encoding c-Met, neuropilin-1 (NRP-1), VEGF-A and VEGF receptor-2 (VEGFR-2) determined by polymerase chain reaction (PCR).

FIG. 17: FOL56 affects the gene expression of cytokines and matrix metalloproteinases in arterial smooth muscle cells. Human coronary artery smooth cells were treated with FOL 56 in concentrations of 10 nM, 100 nM, and 1000 nM and the expression of the genes encoding TNF-α, IL-6, MMP2 and MMP3 determined by polymerase chain reaction (PCR). TNF-α is strong pro-inflammatory cytokine while MMP2 is required for activation of smooth muscle cell proliferation and migration.

FIG. 18. Effect of FOL-026 peptide on endothelial cell function and gene expression. The impact of FOL-026 peptide on human umbilical vascular endothelial cells (HUVECs) proliferation (A) and apoptosis (B) induced with low-oxygen condition was determined by BrdU uptake (n=8-9 per group) and active caspase-3/-7 (n=7-10 per group) separately. The ROS level (C) activated by 50 ug/ml oxidized Low-density Lipoprotein (oxLDL) for 2 hours in cells pre-incubated with FOL-026 peptides was assessed using H2O2 measurement (n=4-8 per group).

FIG. 19. Representative pictures of endothelial migration into an in vitro scratch injury are shown (A) and the wound closure rate (B) in HUVECs stimulated with increasing concentration of FOL-026 peptide was quantified by image J software (n=8-9 per group). Scale bar=150 um.

FIG. 20. Effect of FOL-026 peptide on angiogenesis in vitro and vivo. Representative images of tube formation in HUVECs treated with FOL-026 peptides (A). Quantified measurement of total length (B), total master segments length (C), total branching length (D) and total segments length (E) were performed to evaluate angiogenesis in vitro (n=7 per group for B-E).

FIG. 21. The quantification of vessel density was assessed with CD31+ positive area (A) (n=13 per group). Western blot analysis of phosphorylated AKT (pAKT-T308), AKT, phosphorylated ERK1/2 (p-ERK1/2) and ERK1/2 in endothelial cells stimulated with FOL-26 peptides for 48 hours (B). Quantification of phosphorylation intensities normalized to respective total expressions (C and D, n=3 per group).

FIG. 22. Effect of NRP-1 knock-down on endothelial cell function induced with FOL-026 peptide. The efficiency of small interfering RNA transfection for 48 hours in HUVECs was confirmed by qRT-PCR (n=4) (A). Effect of FOL-026 peptide stimulation on cell proliferation (B) and cell apoptosis at 0.1% oxygen (C) in HUVECs transfected with siRNA-NC or siNRP-1 was evaluated by BrdU incorporation (n=4-7 per group) and active caspase-3/-7 (n=4-7 per group).

FIG. 23. Representative pictures of endothelial cells at 0 and 5 hours after scratch wounding are shown in (A). The wound closure rate (B) of siRNA-NC/siNRP-1-transfected HUVECs stimulated with increasing concentration of FOL-026 peptide was quantified by image J software (n=9-12 per group). Scale bar=150 um.

FIG. 24. Effect of NRP-1 knock-down on tube formation induced with FOL-026 peptide. Tube formation with rising dose of FOL-026 peptide treatment in siRNA-NC or siNRP-1 group (A), total length (B), total master segments length (C), total branching length (D) and total segments length (E) was calculated using Angiogenesis Analyzer plug-in ImageJ (n=7-12 per group). Scale bar=150 um.

FIG. 25. Effect of FOL-026 peptide on smooth muscle cell function. The effect of FOL-026 peptide treatment on smooth muscle cell proliferation (A) and apoptosis (B) induced with low-oxygen condition was evaluated with the uptake of BrdU (n=10 per group) and active caspase-3/-7 (n=6-8 per group) separately. H2O2 measurement (C) of smooth muscle cells pre-incubated with FOL-26 peptides after 50 ug/ml oxidized Low-density Lipoprotein (oxLDL) stimulation for 2 hours (n=6-7 per group).

FIG. 26. Representative images of scratch wound assay in vitro are shown in (A) and the ability of wound healing (B) in human coronary smooth muscle cells (HCASMCs) treated with FOL-026 peptides was measured by image J software (n=10 per group). Scale bar=150 um.

FIG. 27. Effect of NRP-1 knock-down on smooth muscle cell function induced with FOL-026 peptide. qRT-PCR analysis of NRP-1 mRNA expression in HCASMCs with small interfering RNA transfection for 48 hours (n=4) (A). The assessment of BrdU incorporation (B) (n=10-11) and caspase-3/-7 activation (C), (n=3-10). in siRNA-NC/siNRP-1-transfected HUVECs treated with indicated concentration of FOL-026 peptide.

FIG. 28. Representative images of smooth muscle cells at 0 and 5 hours after scratch wounding are shown in (A). The wound closure rate (B) of HCASMCs in siRNA-NC and siNRP-1 group stimulated with increasing concentration of FOL-026 peptide was quantified by image J software (n=9-12 per group). Scale bar=150 um.

FIG. 29. Effect of FOL-005 peptide on endothelial and smooth muscle cell proliferation and wound healing ability. The endothelial cell proliferation (A) was assessed with BrdU uptake in human umbilical vascular endothelial cells (HUVECs) stimulated with increasing concentration of FOL-005 peptide for 48 hours (n=7-10 per group). The wound closure rate (B) in HUVECs with indicated dose of FOL-005 treatment was quantified by image J software (n=12-14 per group) and representative images of at 0 and 6 hours after scratch wounding are shown in (C).

FIG. 30. The effect of FOL-005 peptide on smooth muscle cell proliferation (A) was determined with BrdU incorporation in peptide-treated human coronary smooth muscle cells (HCASMCs).

Scratch wound assay (B) of HCASMCs incubated with FOL-005 peptides for 5 hours was measured by image J software (n=10-12 per group) and representative pictures of smooth muscle cell migration into an in vitro scratch injury are shown in (C). Scale bar =150 um.

FIG. 31. Effect of FOL-005 peptide on tube formation and the role of NRP-1 in that. Representative images of tube formation in human umbilical vascular endothelial cells (HUVECs) treated with FOL-005 peptides are shown in (A and B).

Scale bar=150 um.

### DETAILED DESCRIPTION

### Definitions

As used herein, the singular forms “a”, “an” and “the” include plural referents unless the context clearly states otherwise.

The term “some embodiments” can include one, or more than one embodiment.

The use of the word “a” or “an” when used throughout the text or in conjunction with the term “comprising” in the claims and/or the specification may mean “one,” but it is also consistent with the meaning of “one or more,” “at least one,” and “one or more than one.” “Preventing” or “Prevention” as used herein, includes delaying, stopping, reducing the risk of the onset, of disease, disorder, or condition.

### Agent

The agent of the present invention may be a peptide; a polynucleotide encoding said peptide; a vector comprising said polynucleotide; or a cell comprising said polynucleotide or vector.

### Peptides

In one embodiment, the agent is a peptide or a pharmaceutically acceptable salt thereof.

The term ‘amino acid’ as used herein includes the standard twenty genetically-encoded amino acids and their corresponding stereoisomers in the ‘D’ form (as compared to the natural ‘L’ form), omega-amino acids and other naturally-occurring amino acids, unconventional amino acids (e.g., α,a-disubstituted amino acids, N-alkyl amino acids, etc.) and chemically derivatized amino acids (see below).

When an amino acid is being specifically enumerated, such as ‘alanine’ or ‘Ala’ or ‘A’, the term refers to both L-alanine and D-alanine unless explicitly stated otherwise. Other unconventional amino acids may also be suitable components for peptides of the present disclosure, as long as the desired functional property is retained by the peptide. For the peptides shown, each encoded amino acid residue, where appropriate, is represented by a single letter designation, corresponding to the trivial name of the conventional amino acid.

In one embodiment, the peptide is non-naturally occurring, such as a peptide comprising non-proteinogenic amino acid residues.

In one embodiment, the agent comprises or consists of a tandem repeat comprising two or more repeat units. In one embodiment, the repeat unit comprises or consists of the amino acid sequence of any one or more of the sequences as described herein.

In one embodiment, the peptide is cyclic. The cyclic structure may be achieved by any suitable method of synthesis. Thus, heterodetic linkages may include, but are not limited to formation via disulphide, cysteine, alkylene or sulphide bridges. In one embodiment, the peptide is capable of forming at least one intramolecular cysteine bridge.

In one embodiment, the agent comprises or consists of a peptide comprising or consisting of an amino acid sequence of the general formula:

(SEQ ID NO: 1)
X₅X₆SX₇X₈YGLR

wherein:
   X₅ is D or G;
   X₆ is I or G;
   X₇ is V or L; and
   X₈ is V or A.

In one embodiment, the peptide comprises or consists of an amino acid sequence of the general formula:

(SEQ ID NO: 2)
VDX₂X₃X₄GX₅X₆SX₇X₈YGLR

wherein:
   X₂ is T or V;
   X₃ is Y or P;
   X₄ is D or N;
   X₅ is D or G;
   X₆ is I or G;
   X₇ is V or L; and
   X₈ is V or A.

In one embodiment, the peptide comprises or consists of an amino acid sequence of the general formula:

(SEQ ID NO: 3)
VDTYX₄GX₅X₆SX₇X₈YGLR

wherein:
   X₄ is D or N;
   X₅ is D or G;
   X₆ is I or G;
   X₇ is V or L; and
   X₈ is V or A.

In one embodiment, the peptide comprises or consists of an amino acid sequence of the general formula:

(SEQ ID NO: 4)
VDTYDGZ₇Z₈SZ₁₀Z₁₁YGLR

wherein:
   X₅ is D or G;
   X₆ is I or G;
   X₇ is V or L; and
   X₃ is V or A.

In one embodiment, the peptide comprises or consists of an amino acid sequence of the general formula:

(SEQ ID NO: 5)
VDTYDGZ₇Z₈SVVYGLR

wherein:
   X₅ is D or G; and
   X₆ is I or G;

In one embodiment, the agent comprises or consists of a peptide comprising or consisting of an amino acid sequence of the general formula:

(SEQ ID NO: 105)
X₁₄LX₁₅YGIK

wherein:
   X₁₄ is E or G; and
   X₁₅ is S or T.

In one embodiment, the peptide comprises or consists of an amino acid sequence of the general formula:

(SEQ ID NO: 106)
KX₉LAX₁₀X₁₁X₁₂X₁₃IX₁₄LX₁₅YGIK

wherein:
   X₉ is C, P or G;
   X₁₀ is E or G;
   X₁₁ is C, D or I;
   X₁₂ is D, I, S or G;
   X₁₃ is S, D or G;
   X₁₄ is E or G; and
   X₁₅ is S or T.

In one embodiment, the peptide comprises or consists of an amino acid sequence of the general formula:

(SEQ ID NO: 107)
KX₉LAX₁₀X₁₁X₁₂X₁₃IX₁₄LSYGIK

wherein:
   X₉ is C, P or G;
   X₁₀ is E or G;
   X₁₁ is C, I or absent;
   X₁₂ is D, G or absent;
   X₁₃ is S, G or absent; and
   X₁₄ is E or G.

In one embodiment, the peptide comprises or consists of an amino acid sequence of the general formula:

(SEQ ID NO: 108)
KX₉LAX₁₀IX₁₄LSYGIK

wherein:
   X₉ is C, P or G;
   X₁₀ is E or G; and
   X₁₄ is E or G.

In one embodiment, the peptide comprises or consisted of the amino acid sequence IELSYGIK (SEQ ID NO: 109).

In one embodiment, the peptide comprises or consists of VDTYDGGISVVYGLR (SEQ ID NO: 6). In one embodiment, the peptide comprises or consists of AEIDSIELSYGIK (SEQ ID NO: 110). In one embodiment, the peptide comprises or consists of VDTYDGDISVVYGLR (SEQ ID NO: 7). In one embodiment, the peptide comprises or consists of DTYDGDISVVYGLR (SEQ ID NO: 8). In one embodiment, the peptide comprises or consists of TYDGDISVVYGLRS (SEQ ID NO: 9). In one embodiment, the peptide comprises or consists of TYDGDISVVYGLR (SEQ ID NO: 10). In one embodiment, the peptide comprises or consists of YDGDISWYGLRS (SEQ ID NO: 11). In one embodiment, the peptide comprises or consists of YDGDISVVYGLR (SEQ ID NO: 12). In one embodiment, the peptide comprises or consists of DGDISVVYGLRS (SEQ ID NO: 13). In one embodiment, the peptide comprises or consists of DGDISVVYGLR (SEQ ID NO: 14). In one embodiment, the peptide comprises or consists of GDISVVYGLRS (SEQ ID NO: 15). In one embodiment, the peptide comprises or consists of GDISVVYGLR (SEQ ID NO: 16). In one embodiment, the peptide comprises or consists of DISVVYGLRS (SEQ ID NO: 17). In one embodiment, the peptide comprises or consists of DISVVYGLR (SEQ ID NO: 18). In one embodiment, the peptide comprises or consists of VDVPNGDISLAYGLR (SEQ ID NO: 19). In one embodiment, the peptide comprises or consists of DVPNGDISLAYGLRS (SEQ ID NO: 20). In one embodiment, the peptide comprises or consists of DVPNGDISLAYGLR (SEQ ID NO: 21). In one embodiment, the peptide comprises or consists of VPNGDISLAYGLRS (SEQ ID NO: 22). In one embodiment, the peptide comprises or consists of VPNGDISLAYGLR (SEQ ID NO: 23). In one embodiment, the peptide comprises or consists of PNGDISLAYGLRS (SEQ ID NO: 24). In one embodiment, the peptide comprises or consists of PNGDISLAYGLR (SEQ ID NO: 25). In one embodiment, the peptide comprises or consists of NGDISLAYGLRS (SEQ ID NO: 26). In one embodiment, the peptide comprises or consists of NGDISLAYGLR (SEQ ID NO: 27). In one embodiment, the peptide comprises or consists of GDISLAYGLRS (SEQ ID NO: 28). In one embodiment, the peptide comprises or consists of GDISLAYGLR (SEQ ID NO: 29). In one embodiment, the peptide comprises or consists of DISLAYGLRS (SEQ ID NO: 30). In one embodiment, the peptide comprises or consists of DISLAYGLR (SEQ ID NO: 31). In one embodiment, the peptide comprises or consists of VDTYDGDGSVVYGLR (SEQ ID NO: 32). In one embodiment, the peptide comprises or consists of VDVPEGDISLAYGLR (SEQ ID NO: 33). In one embodiment, the peptide comprises or consists of KPLAEIDSIELSYGIK (SEQ ID NO: 111). In one embodiment, the peptide comprises or consists of KCLAECDSIELSYGIK (Cyclic) (SEQ ID NO: 112). In one embodiment, the peptide comprises or consists of KPLAEDISIELSYGIK (SEQ ID NO: 113). In one embodiment, the peptide comprises or consists of KPLAEISDIELSYGIK (SEQ ID NO: 114). In one embodiment, the peptide comprises or consists of KPLAEIGDIELSYGIK (SEQ ID NO: 115). In one embodiment, the peptide comprises or consists of KPLAEGDIELSYGIK (SEQ ID NO: 116). In one embodiment, the peptide comprises or consists of KPLAEIELSYGIK (SEQ ID NO: 117). In one embodiment, the peptide comprises or consists of KPLAEIDSIELTYGIK (SEQ ID NO: 118). In one embodiment, the peptide comprises or consists of KPLAEIDGIELSYGIK (SEQ ID NO: 119). In one embodiment, the peptide comprises or consists of KPLAEIDGIELTYGIK (SEQ ID NO: 120). In one embodiment, the peptide comprises or consists of KPLAEIGSIELSYGIK (SEQ ID NO: 121). In one embodiment, the peptide comprises or consists of KGLAEIDSIELSYGIK (SEQ ID NO: 122). In one embodiment, the peptide comprises or consists of KPLAGIDSIGLSYGIK (SEQ ID NO: 123). In one embodiment, the peptide comprises or consists of Cyclic KCLAEIDSCELSYGIK (SEQ ID NO: 124). In one embodiment, the peptide comprises or consists of LAEIDSIELSYGIK (SEQ ID NO: 125). In one embodiment, the peptide comprises or consists of EIDSIELSYGIK (SEQ ID NO: 126).

In one embodiment, the peptide comprises or consists of IDSIELSYGIK (SEQ ID NO: 127). In one embodiment, the peptide comprises or consists of DSIELSYGIK (SEQ ID NO: 128). In one embodiment, the peptide comprises or consists of SIELSYGIK (SEQ ID NO: 129). In one embodiment, the peptide comprises or consists of IELSYGIK (SEQ ID NO: 109). In one embodiment, the peptide comprises or consists of VDTYDGDISVVYGL (SEQ ID NO: 34). In one embodiment, the peptide comprises or consists of VDTYDGDISVVYG (SEQ ID NO: 35). In one embodiment, the peptide comprises or consists of VDTYDGDISVVY (SEQ ID NO: 36). In one embodiment, the peptide comprises or consists of VDTYDGDISVV (SEQ ID NO: 37). In one embodiment, the peptide comprises or consists of VDTYDGDISV (SEQ ID NO: 38). In one embodiment, the peptide comprises or consists of VDTYDGDIS (SEQ ID NO: 39). In one embodiment, the peptide comprises or consists of VDTYDGRGDSVVYGLR (SEQ ID NO: 40). In one embodiment, the peptide comprises or consists of VDVPNGDISLAYGL (SEQ ID NO: 41). In one embodiment, the peptide comprises or consists of VDVPNGDISLAYG (SEQ ID NO: 42). In one embodiment, the peptide comprises or consists of VDVPNGDISLA (SEQ ID NO: 43). In one embodiment, the peptide comprises or consists of VDVPNGDIS (SEQ ID NO: 44). In one embodiment, the peptide comprises or consists of GDPNDGRGDSVVYGLR (SEQ ID NO: 45 In one embodiment, the peptide comprises or consists of LDGLVRAYDNISPVG (SEQ ID NO: 46). In one embodiment, the peptide comprises or consists of GDPNGDISVVYGLR (SEQ ID NO: 47). In one embodiment, the peptide comprises or consists of VDVPNGDISLAYRLR (SEQ ID NO: 48). In one embodiment, the peptide comprises or consists of VDVPEGDISLAYRLR (SEQ ID NO: 49). In one embodiment, the peptide comprises or consists of V(beta-D)TYDGDISVVYGLR (SEQ ID NO: 50). In one embodiment, the peptide comprises or consists of VDTY(beta-D)GDISVVYGLR (SEQ ID NO: 51 In one embodiment, the peptide comprises or consists of VDTYDG(beta-D)ISVVYGLR (SEQ ID NO: 52). In one embodiment, the peptide comprises or consists of CLAEIDSC (Cyclic) (SEQ ID NO: 130). In one embodiment, the peptide comprises or consists of CFKPLAEIDSIECSYGIK (Cyclic) (SEQ ID NO: 131). In one embodiment, the peptide comprises or consists of Cyclic CFKPLAEIDSIEC (SEQ ID NO: 132). In one embodiment, the peptide comprises or consists of KPLAEIDSIELSYGI (SEQ ID NO: 133). In one embodiment, the peptide comprises or consists of KPLAEIDSIELSYG (SEQ ID NO: 134). In one embodiment, the peptide comprises or consists of KPLAEIDSIELSY (SEQ ID NO: 135), KPLAEIDSIELS (SEQ ID NO: 136). In one embodiment, the peptide comprises or consists of KPLAEIDSIEL (SEQ ID NO: 137). In one embodiment, the peptide comprises or consists of KPLAEIDSIE (SEQ ID NO: 138).

### Salts and Prodrugs

The agent as defined herein can be in the form of a pharmaceutically acceptable salt or prodrug of said agent. In one embodiment, the agent as defined herein can be formulated as a pharmaceutically acceptable addition salt or hydrate of said agent, such as but not limited to K⁺, Na⁺, as well as non-salt e.g. H⁺.

### Modifications

In one embodiment, the agent is a chemical derivative of a peptide. Chemical derivatives of one or more amino acids may be achieved by reaction with a functional side group. Such derivatives include, for example, those molecules in which free amino groups have been derivatized to form amine hydrochlorides, p-toluene sulphonyl groups, carboxybenzoxy groups, t-butyloxycarbonyl groups, chloroacetyl groups or formyl groups. Free carboxyl groups may be derivatized to form salts, methyl and ethyl esters or other types of esters and hydrazides. Free hydroxyl groups may be derivatized to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g. acetylation or thioglycolic acid amidation), terminal carboxylamidation (e.g. with ammonia or methylamine), and the like terminal modifications.

In one embodiment, the agent is further modified such by glycosylation, PEGylation, amidation, esterification, acylation, acetylation and/or alkylation.

In some embodiments, the agent is further conjugated to a moiety, which may be selected from the group consisting of polyethylene glycol (PEG), monosaccharides, fluorophores, chromophores, radioactive compounds, and cell-penetrating peptides. In one embodiment, the fluorophore is selected from the group consisting of Lucifer yellow, biotin, 5,6-carboxyltetramethylrhodamine (TAMRA), indodicarbocyanine (C5) Alexa Fluor®488, Alexa Fluor®532, Alexa Fluor®647, ATTO 488, ATTO 532, 6-carboxyfluorescein (6-FAM), Alexa Fluor®350, DY-415, ATTO 425, ATTO 465, Bodipy® FL, fluorescein isothiocyanate, Oregon Green® 488, Oregon Green® 514, Rhodamine Green™, 5′-Tetrachloro-Fluorescein, ATTO 520, 6-carboxy-4′,5′-dichloro-2′,7′-dimethoxyfluoresceine, Yakima Yellow™ dyes, Bodipy® 530/550, hexachloro-fluorescein, Alexa Fluor®555, DY-549, Bodipy® TMR-X, cyanine phosphoramidites (cyanine 3, cyanine 3.5, cyanine 5, cyanine 5.5, cyanine 7.5), ATTO 550, Rhodamine Red™, ATTO 565, Carboxy-X-Rhodamine, Texas Red (Sulforhodamine 101 acid chloride), LightCycler® Red 610, ATTO 594, DY-480-XL, DY-610, ATTO 610, LightCycler® Red 640, Bodipy 630/650, ATTO 633, Bodipy 650/665, ATTO 647N, DY-649, LightCycler® Red 670, ATTO 680, LightCycler® Red 705, DY-682, ATTO 700, ATTO 740, DY-782, IRD 700, IRD 800, CAL Fluor® Gold 540 nm, CAL Fluor® Gold 522 nm, CAL Fluor® Gold 544 nm, CAL Fluor® Orange 560 nm, CAL Fluor® Orange 538 nm, CAL Fluor® Orange 559 nm, CAL Fluor® Red 590 nm, CAL Fluor® Red 569 nm, CAL Fluor® Red 591 nm, CAL Fluor® Red 610 nm, CAL Fluor® Red 590 nm, CAL Fluor® Red 610 nm, CAL Fluor® Red 635 nm, Quasar® 570 nm, Quasar® 548 nm, Quasar® 566 nm (Cy 3), Quasar® 670 nm, Quasar® 647 nm, Quasar® 670 nm, Quasar® 705 nm, Quasar® 690 nm, Quasar® 705 nm (Cy 5.5), Pulsar® 650 Dyes, SuperRox® Dyes.). In one embodiment, the agent further comprises a detectable moiety, such as a moiety that is detectable by an imaging technique such as SPECT, PET, MRI, optical or ultrasound imaging. In one embodiment, the detectable moiety comprises or consists of a radioisotope, such as selected from the group consisting of ^{99m}Tc, ¹¹¹In, ⁶⁷Ga, ⁶⁸Ga, ⁷²As, ⁸⁹Zr, ¹²³I and ²⁰¹Tl.

In one embodiment, the peptide comprises or consists of a fusion. For example, the peptide may comprise a fusion of two amino acid sequences as disclosed herein.

The term ‘fusion’ of a peptide relates to an amino acid sequence fused to another peptide. For example, the said peptide may be fused to a polypeptide such as glutathione-S-transferase (GST) or protein A in order to facilitate purification of said peptide. Examples of such fusions are well known to those skilled in the art. Similarly, the said peptide may be fused to an oligo-histidine tag such as His6 or to an epitope recognised by an antibody such as the well-known Myc tag epitope. Fusions to any variant or derivative of said peptide are also included in the scope of the disclosure. Alternatively, the fused portion may be a lipophilic molecule or peptide domain that is capable of promoting cellular uptake of the polypeptide, as known to those skilled in the art.

### Peptide Length

In one embodiment, said peptide is of no more than no more than 85, such as no more than 80, such as no more than 75, such as no more than 70, such as no more than 65, such as no more than 60, such as nor more than 55, such as no more than 50, such as no more than 55, such as no more than 40 amino acids, such as no more than 35, such as no more than 30, such as no more than 28, such as no more than 26, such as no more than 24, such as no more than 22, such as no more than 20, such as no more than 19, such as no more than 18, such as no more than 17, such as no more than 16, such as no more than 15, such as no more than 14, such as no more than 13, such as no more than 12, such as no more than 11, such as no more than 10 amino acids in length.

In one embodiment, the peptide comprises at least 2 additional amino acids, such as at least 3, such as at least 4, such as at least 5, such as at least 6, such as at least 7, such as at least 8, such as at least 9, such as at least 10, such as at least 15 or such as at least 20 amino acids conjugated to the N- or C-terminus of the peptide.

In one embodiment, said peptide is between 5 and 30 amino acids in length, such as between 5 and 20, such as between 8 and 20, such as between 8 and 18, such as between 10 and 16 amino acids in length.

In one embodiment, when X₁₄ is T, then the peptide comprises no more than 25 amino acid residues.

In yet another embodiment, the agent is a fragment of a peptide described herein, and the fragment comprises 15 or fewer amino acids in length, such as fewer than 14 amino acids, such as fewer than 13 amino acids, such as fewer than 12 amino acids, such as fewer than 11 amino acids, such as fewer than 10 amino acids, such as fewer than 9 amino acids, such as fewer than 8 amino acids, such as fewer than 7 amino acids, such as fewer than 6 amino acids, such as fewer than 5 amino acids in length.

By “fragment”, at least 5 contiguous amino acids of the amino acid sequence are included, for example at least 6, 7, 8, 9, 10, 11, 12, 13, 14 or 15 contiguous amino acids of the amino acid sequence. The fragment may be 15 or fewer amino acids in length, for example 14, 13, 12, 11, 10, 9, 8, 7, 6 or 5 amino acids in length.

### Variants

In one embodiment, the agent is a variant of a peptide as described herein, wherein the variant comprises or consists of a sequence wherein any one amino acid has been altered for another proteinogenic or non-proteinogenic amino acid, with the proviso that no more than five amino acids are so altered.

The term “variant” refers to a peptide that does not share 100% amino acid sequence identity with the parent peptide, i.e. one or more amino acids must be mutated. “Mutated” refers to altering an amino acid at a specified position in the parent peptide. For example, an amino acid at a specified position may be deleted, altered, substituted or may be the site of an insertion/addition of one or more amino acids. It will be appreciated by persons skilled in the art that the substitutions may be conservative or non-conservative.

In one embodiment, said peptide variant comprises or consists of a sequence wherein no more than five amino acids are altered for another proteinogenic or non-proteinogenic amino acid, such as no more than 4 amino acids, such as no more than 3 amino acids, such as no more than 2 amino acids, such as no more than 1 amino acid is altered. In one embodiment, one or more amino acids are conservatively substituted. “Conservatively substituted” refers to a substitution of one amino acid with another with similar properties (size, hydrophobicity, etc.), such that the function of the peptide is not significantly altered. Thus, by “conservative substitutions” is intended combinations such as Gly, Ala; Val, Ile, Leu; Asp, Glu; Asn, Gln; Ser, Thr; Lys, Arg; and Phe, Tyr.

In one embodiment, the peptide has one additional amino acid. In one embodiment, said peptide comprises or consists of one or more additional amino acids, inserted at the N- and/or C-terminus and/or internally within the sequence. In one embodiment, at least 2 additional amino acids, such as at least 3, such as at least 4, such as at least 5, such as at least 6, such as at least 7, such as at least 8, such as at least 9, such as at least 10, such as at least 15 or such as at least 20 additional amino acids are inserted.

In one embodiment, the agent increases angiogenesis in the subject. In one embodiment, the agent is an angiogenesis inducer. In one embodiment, the agent is capable of improving myocyte survival in cardiovascular disease. In one embodiment, the agent is capable of improving neural cell survival in cerebrovascular disease.

### Polynucleotides, Vectors and Cells

In embodiment, the agent is a polynucleotide encoding upon expression a peptide as described herein. In embodiment, the agent is a vector comprising a polynucleotide as described herein. In embodiment, the agent is a cell comprising a polynucleotide or a vector as described herein.

### Compositions and Coatings

In one embodiment, the agent is in a composition. In one embodiment, the composition is a pharmaceutical composition. In one embodiment, the composition is a cosmetic composition. In one embodiment, the composition is a coating.

Coatings on various implants are known in the art. Applications in humans include central venous catheters, coronary stents, ventricular assist devices, extracorporeal blood circuits, blood sampling devices, and vascular grafts. Such coatings can be in a gel or non-gel form. As used herein, a “coating comprising the agent” includes that the agent adsorbed to the surface, bonded to the surface, and imbedded in the polymer surface.

### Implants

In one aspect, the present invention relates to an implant comprising the agent described herein. In one embodiment, the implant is coated with a composition comprising the agent. Thus, the agent may for example be adsorbed to the surface, bonded to the surface, and imbedded in the polymer surface of the implant.

In one embodiment, the implant is of a biomaterial, such as bone.

In one embodiment, the implant is a medical device, such as a stent.

### Medical Use

The agents disclosed herein have been shown to induce angiogenesis. This property can be employed to treat various diseases and disorders. Such diseases and disorders will typically be associated with an abnormal level of angiogenesis, such as reduced angiogenesis compared to healthy tissue.

One embodiment of the present disclosure provides for the agent as disclosed herein, for use in increasing angiogenesis in a subject.

One embodiment of the present disclosure provides for the agent as disclosed herein, for use as an angiogenesis inducer.

It may be desired to increase angiogenesis in the myocardium, e.g. by increasing angiogenesis in affected tissues. Various cardiovascular diseases are associated with ischaemia, e.g. reduced oxygen supply to affected tissues. Thus, one embodiment of the present disclosure provides for the agent as disclosed herein, for use in improving myocyte survival in cardiovascular disease.

Certain cerebrovascular diseases are associated with reduced oxygen flow to affected tissues. It may be beneficial increasing vascularisation to affected tissues, e.g. to improve oxygen supply. One embodiment of the present disclosure provides for the agent as disclosed herein, for use in improving neural cell survival in cerebrovascular disease.

One embodiment of the present disclosure provides for the agent as disclosed herein for use in the treatment of a disease or disorder associated with reduced or impaired angiogenesis.

One embodiment of the disclosure provides for a method for inducing vascularization, said method comprising administering the agent of the disclosure to a subject.

Many diseases and disorders are associated with reduced or impaired vascularisation, or can advantageously be ameliorated by increasing vascularisation in the affected tissues. One embodiment of the present disclosure provides for the agent as disclosed herein for use in the treatment or prevention of a disease or disorder in a subject, wherein the disease or disorder is selected from the group consisting of:
i. a disease of the circulatory system,
ii. an injury of external cause,
iii. a disease of the immune system,
iv. a disease of the nervous system, and
v. a disease of the musculoskeletal system or connective tissue.

Diseases of the circulatory system refers to diseases of the organ system that passes nutrients (such as amino acids, electrolytes and lymph), gases, hormones, blood cells, etc. to and from cells in the body to help fight diseases, stabilize body temperature and pH, and to maintain homeostasis. An injury of external cause means physical or physiological bodily harm resulting from interaction of the body with energy (mechanical, thermal, electrical, chemical or radiant, or due to extreme pressure) in an amount, or at a rate of transfer, that exceeds physical or physiological tolerance. Injury can also result from lack of vital elements, such as oxygen. Poisoning by and toxic effects of substances are included, as is damage of or due to implanted devices. Injury usually has rapid onset in response to a well-defined event (e.g. a car crash, striking the ground after falling, drinking a strongly alkaline liquid, an overdose of a medication, a burn sustained during a surgical procedure). These events are often referred to as external causes of injury. The injurious energy can, however, originate from the injured person and/or from his or her immediate environment (e.g. a person running on a hot day sustains heat exhaustion), and injury can be caused by the injured person (i.e. intentional self-harm). Injury includes manifestations that are evident immediately after onset, which may persist or not, and manifestations that first become evident at a later date.

In one embodiment, the disease or disorder associated with reduced or impaired angiogenesis is a complication associated with a disease or disorder selected from the group consisting of:
i. a disease of the circulatory system,
ii. an injury of external cause,
iii. a disease of the immune system,
iv. a symptom, sign, or clinical finding of the circulatory system,
v. a disease of the myocardium or cardiac chambers, and
vi. an endocrine, nutritional or metabolic disease.

One embodiment of the present disclosure provides for the agent as disclosed herein for use in the treatment or prevention of a complication associated with a disease or disorder selected from the group consisting of:
vii. a disease of the circulatory system,
viii. an injury of external cause,
ix. a disease of the immune system,
x. a symptom, sign, or clinical finding of the circulatory system,
xi. a disease of the myocardium or cardiac chambers, and
xii. an endocrine, nutritional or metabolic disease.

In one embodiment of the present disclosure the disease of the circulatory system is selected from the group consisting of:
i. an ischaemic heart disease,
ii. a cerebrovascular disease,
iii. a disease of coronary artery,
iv. a disease of the arteries, arterioles, or capillaries,
v. a symptom, sign, or clinical finding of the circulatory system,
vi. a disease of the myocardium or cardiac chambers, and
vii. an endocrine, nutritional or metabolic disease.

Cerebrovascular diseases are a group of brain dysfunctions related to disease of the blood vessels supplying the brain. This includes “stroke”, which includes the following entities—. Intracerebral haemorrhage; Subarachnoid haemorrhage; cerebral ischemic stroke, and Stroke not known if ischaemic or haemorrhagic.

Clinical findings of the circulatory system include those found using physical, laboratory and imaging techniques. Diseases can manifest in many ways and in different body systems. Such specific manifestations may be a reason for treatment or encounter, with or without identifying or addressing the underlying condition. This includes the less well-defined conditions and symptoms that, without the necessary study of the case to establish a final diagnosis, could be designated ‘not otherwise specified’, ‘unknown aetiology’ or ‘transient’. The conditions and signs or symptoms included consist of: cases for which no more specific diagnosis can be made even after all the facts bearing on the case have been investigated; signs or symptoms existing at the time of initial encounter that proved to be transient and whose causes could not be determined; provisional diagnoses in a patient who failed to return for further investigation or care; cases referred elsewhere for investigation or treatment before the diagnosis was made; cases in which a more precise diagnosis was not available for any other reason; certain symptoms, for which supplementary information is provided, that represent important problems in medical care in their own right.

Diseases of the myocardium or cardiac chambers include diseases of a type of involuntary striated muscle found in the walls and histological foundation of the heart, with specific reference to the atrial and ventricular chambers, as well as the myocardium itself.

In one embodiment of the present disclosure, the ischaemic heart disease is selected from the group consisting of:
i. acute ischaemic heart disease, such as myocardial infarction, such as acute myocardial infarction and unspecified acute ischaemic heart disease (acute coronary syndrome),
ii. chronic ischaemic heart disease, such as other specific chronic ischaemic heart disease (cardiovascular arteriosclerosis),
iii. angina pectoris, such as unstable angina pectoris, and
iv. obstructive arteriosclerosis.

Chronic heart disease is seen due to the atherosclerosis of coronary arteries. It is characterised by angina pectoris and unstable angina.

In one embodiment, the myocardial infarction is ST elevation myocardial infarction (STEMI) or non-ST elevation myocardial infarction (NSTEMI). In one embodiment the STEMI or NSTEMI presents with subsequent certain current complications, such as within a 28 day period.

In one embodiment the disease of the circulatory system is cardiovascular sclerosis. In one embodiment, the disease of the circulatory system is systemic sclerosis or associated with systemic sclerosis.

In one embodiment the cerebrovascular disease is selected from the group consisting of:
i. cerebral ischaemia, such as cerebral ischaemic stroke (stroke), such as cerebral infarction, and
ii. asymptomatic stenosis of intracranial or extracranial artery (cerebral arteriosclerosis).

Acute focal neurological dysfunction caused by focal infarction at single or multiple sites of the brain. Evidence of acute infarction may come either from a) symptom duration lasting more than 24 hours, or b) neuroimaging or other technique in the clinically relevant area of the brain.

In one embodiment, the cerebral ischaemic stroke is associated with a patient history of transient ischaemic attack (TIA) or cerebral infarction without residual deficits. Transient episode of focal neurological dysfunction caused by focal brain ischemia without acute infarction in the clinically relevant area of the brain or transient monocular visual loss due to retinal ischemia. Symptoms should resolve completely within 24 hours.

In one embodiment the cerebral ischaemic stroke is associated with a patient history of traumatic brain injury or sequelae of cerebrovascular disease.

In one embodiment, the cerebrovascular disease is selected from the group consisting of:
i. nontraumatic subarachnoid haemorrhage,
ii. nontraumatic intracerebral haemorrhage,
iii. other and unspecified nontraumatic intracranial haemorrhage,
iv. cerebral infarction,
v. occlusion and stenosis of precerebral arteries not resulting in cerebral infarction,
vi. occlusion and stenosis of cerebral arteries, not resulting in cerebral infarction,
vii. other cerebrovascular diseases,
viii. cerebrovascular disorder associated with other diseases (cerebrovascular disorders classified elsewhere), and
ix. sequelae of cerebrovascular disease.

In one embodiment, the disease of the arteries, arterioles, or capillaries is selected from the group consisting of:
i. atherosclerosis,
ii. aortic aneurysm and dissection,
iii. an other aneurysm,
iv. an other peripheral vascular disease,
v. arterial embolism and thrombosis,
vi. atheroembolism,
vii. septic arterial embolism,
viii. an other disorder of arteries and arterioles,
ix. a disease of the capillaries, and
x. a disorder of the arteries, arterioles, or capillaries associated with another disease (a disorder of the arteries, arterioles, or capillaries in a disease classified elsewhere).

In one embodiment, the disease of the arteries, arterioles, or capillaries is chronic arterial occlusive disease, such as atherosclerotic chronic arterial occlusive disease or vascular sclerosis.

In one embodiment, the symptom, sign, or clinical finding of the circulatory system is a symptom or sign involving the circulatory system, such as an abnormal blood-pressure reading without diagnosis, such as cardiac arrest.

In one embodiment the disease of the myocardium or cardiac chambers is cardiomyopathy, such as dilated cardiomyopathy. These are myocardial disorders in which the heart muscle is structurally and functionally abnormal, in the absence of coronary artery disease, hypertension, valvular disease and congenital heart disease sufficient to cause the observed myocardial abnormality.

In one embodiment, the disease of the immune system is non-organ specific systemic autoimmune disorder, such as vasculitis, such as thromboangiitis obliterans (Buerger's disease).

In one embodiment, the disease of the nervous system is selected from the group consisting of:
i. a movement disorder, such as parkinsonism, such as Parkinson's disease,
ii. multiple sclerosis or other white matter disorders, such as multiple sclerosis, and
iii. disorders with neurocognitive impairment as a major feature, such as Alzheimer's disease.

In one embodiment, the disease of the musculoskeletal system or connective tissue is a condition associated with the spine, such as herniated disc.

In one embodiment, the disease of the circulatory system, the disease of the immune system, the disease of the nervous system, or the disease of the musculoskeletal system or connective tissue is or is associated with diabetes mellitus. In one embodiment, the diabetes mellitus is selected from Type 1 diabetes mellitus and Type 2 diabetes mellitus. In one embodiment, the subject is suffering from diabetes mellitus.

In one embodiment, the subject is a mammal. In one embodiment, the mammal is a human.

One embodiment of the present disclosure provides for a method for treating or preventing a disease or disorder in a subject, wherein the disease or disorder is selected from the group consisting of:
i. a disease of the circulatory system,
ii. an injury of external cause,
iii. a disease of the immune system,
iv. a disease of the nervous system, and
v. a disease of the musculoskeletal system or connective tissue,
the method comprising administering a therapeutically effective amount of an agent as defined herein to an subject in need thereof.

On embodiment of the disclosure provides for a use of the agent as defined herein for the manufacture of a medicament for the treatment or prevention of a disease or disorder selected from the group consisting of:
i. a disease of the circulatory system,
ii. an injury of external cause,
iii. a disease of the immune system,
iv. a disease of the nervous system, and
v. a disease of the musculoskeletal system or connective tissue,

One aspect of the present disclosure provides for the agent as disclosed herein, for use in increasing angiogenesis in a subject.

One aspect of the present disclosure provides for the agent as disclosed herein, for use as an angiogenesis inducer.

One aspect of the present disclosure provides for the agent as disclosed herein for use in the treatment of a disease or disorder associated with reduced or impaired angiogenesis.

One aspect of the disclosure provides for a method for inducing vascularization, said method comprising administering the agent of the disclosure to a subject.

One aspect of the present disclosure provides for the agent as disclosed herein for use in the treatment or prevention of a disease or disorder in a subject, wherein the disease or disorder is selected from the group consisting of:
i. a disease of the circulatory system,
ii. an injury of external cause,
iii. a disease of the immune system,
iv. a disease of the nervous system, and
v. a disease of the musculoskeletal system or connective tissue.

One aspect of the present disclosure provides for the agent as disclosed herein for use in the treatment or prevention of a complication associated with a disease or disorder selected from the group consisting of:
i. a disease of the circulatory system,
ii. an injury of external cause,
iii. a disease of the immune system,
iv. a symptom, sign, or clinical finding of the circulatory system,
v. a disease of the myocardium or cardiac chambers, and
vi. an endocrine, nutritional or metabolic disease.

### Surgery, Implants, and Transplants

One embodiment of the present disclosure provides for the agent of the disclosure, for use in improving vascularisation post surgery. In one embodiment, the surgery is surgery of the cardiovascular system. In one embodiment, the surgery of the cardiovascular system is a vascular graft. In one embodiment, the implant is of a biomaterial. In one embodiment the biomaterial is bone. In one embodiment, the implant is a medical device. In one embodiment, the medical device is a stent.

One embodiment of the disclosure provides for the disclosed agent for use in increasing angiogenesis in a subject undergoing or having undergone transplant. In one embodiment, the transplant is of an organ, of a tissue, or of a cell. In one embodiment, the transplant of a cell is of bone marrow cells. In one embodiment, the transplant of an organ is of a heart or a cardiovascular tissue.

### Items

1. An agent comprising:
   a) a peptide selected from the group consisting of:
      (i) a peptide comprising or consisting of an amino acid sequence of the general formula:

(SEQ ID NO: 1)
X₅X₆SX₇X₈YGLR

wherein:
   X₅ is D or G;
   X₆ is I or G;
   X₇ is V or L;
   X₈ is V or A;
(ii) a peptide comprising or consisting of an amino acid sequence of the general formula:

(SEQ ID NO: 105)
X₁₄LX₁₅YGIK

wherein:
   X₁₄ is E or G;
   X₁₅ is S or T;
(iii) a peptide comprising or consisting of an amino acid sequence selected from the group consisting of:

(SEQ ID NO: 34)
VDTYDGDISVVYGL,
(SEQ ID NO: 35)
VDTYDGDISVVYG,
(SEQ ID NO: 36)
VDTYDGDISVVY,
(SEQ ID NO: 37)
VDTYDGDISVV,
(SEQ ID NO: 38)
VDTYDGDISV,
(SEQ ID NO: 39)
VDTYDGDIS,
(SEQ ID NO: 40)
VDTYDGRGDSVVYGLR,
(SEQ ID NO: 41)
VDVPNGDISLAYGL,
(SEQ ID NO: 42)
VDVPNGDISLAYG,
(SEQ ID NO: 43)
VDVPNGDISLA,
(SEQ ID NO: 44)
VDVPNGDIS,
(SEQ ID NO: 45)
GDPNDGRGDSVVYGLR,
(SEQ ID NO: 46)
LDGLVRAYDNISPVG,
(SEQ ID NO: 47)
GDPNGDISVVYGLR
(SEQ ID NO: 48)
VDVPNGDISLAYRLR,
(SEQ ID NO: 49)
VDVPEGDISLAYRLR,
(SEQ ID NO: 50)
V(beta-D)TYDGDISVVYGLR,
(SEQ ID NO: 51)
VDTY(beta-D)GDISVVYGLR,
(SEQ ID NO: 52)
VDTYDG(beta-D)ISVVYGLR;
(SEQ ID NO: 130)
CLAEIDSC (Cyclic),
(SEQ ID NO: 131)
CFKPLAEIDSIECSYGIK (Cyclic),
(SEQ ID NO: 132)
Cyclic CFKPLAEIDSIEC,
(SEQ ID NO: 133)
KPLAEIDSIELSYGI,
(SEQ ID NO: 134)
KPLAEIDSIELSYG,
(SEQ ID NO: 135)
KPLAEIDSIELSY,
(SEQ ID NO: 136)
KPLAEIDSIELS,
(SEQ ID NO: 137)
KPLAEIDSIEL,
and
(SEQ ID NO: 138)
KPLAEIDSIE;

b) a polynucleotide encoding upon expression, the peptide of a);
c) a vector comprising the polynucleotide of b); or
d) a cell comprising the polynucleotide of b), or the vector of c).
2. The agent according to item1, wherein the peptide comprises an amino acid sequence of the general formula:

(SEQ ID NO: 2)
VDX₂X₃X₄GX₅X₆SX₇X₈YGLR

wherein:
X₂ is T or V;
X₃ is Y or P;
X₄ is D or N;
X₅ is D or G;
X₆ is I or G;
X₇ is V or L; and
X₈ is V or A.
3. The agent according to any one of the preceding items, wherein the peptide comprises an amino acid sequence of the general formula:

(SEQ ID NO: 3)
VDTYX₄GX₅X₆SX₇X₈YGLR

wherein:
X₄is D or N;
X₅ is D or G;
X₆ is I or G;
X₇ is V or L; and
X₈ is V or A.
4. The agent according to any one of the preceding items, wherein the peptide comprises an amino acid sequence of the general formula:

(SEQ ID NO: 4)
VDTYDGZ₇Z₈SZ₁0Z₁₁YGLR

wherein:
X₅ is D or G;
X₆ is I or G;
X₇ is V or L; and
X₈ is V or A.
5. The agent according to any one of the preceding items, wherein the peptide comprises an amino acid sequence of the general formula:

(SEQ ID NO: 5)
VDTYDGZ₇Z₈SVVYGLR

wherein:
X₅ is D or G; and
X₆ is I or G;
6. The agent according to item 1, wherein the peptide comprises an amino acid sequence of the general formula:

(SEQ ID NO: 106)
KX₉LAX₁₀X₁₁X₁₂X₁₃IX₁₄LX₁₅YGIK

wherein:
X₉ is C, P or G;
X₁₀ is E or G;
X₁₁ is C, D or I;
X₁₂ is D, I, S or G;
X₁₃ is S, D or G;
X₁₄ is E or G; and
X₁₅ is S or T.
7. The agent according to item 1, wherein the peptide comprises an amino acid sequence of the general formula:

(SEQ ID NO: 107)
KX₉LAX₁₀X₁₁X₁₂X₁₃IX₁₄LSYGIK

wherein:
X₉ is C, P or G;
X₁₀ is E or G;
X₁₁ is C, I or absent;
X₁₂ is D, G or absent;
X₁₃ is S, G or absent; and
X₁₄ is E or G.
8. The agent according to item 1, wherein the peptide comprises an amino acid sequence of the general formula:

(SEQ ID NO: 108)
KX₉LAX₁₀IX₁₄LSYGIK

wherein:
X₉ is C, P or G;
X₁₀ is E or G; and
X₁₄ is E or G.
9. The agent according to item 1, wherein the peptide comprises the amino acid sequence IELSYGIK (SEQ ID NO: 109).
10. The agent according to item 1, with the proviso that if X₁₄ is T, the peptide comprises no more than 25 amino acid residues.
11. The agent according to item 1, wherein the peptide comprises or consists of an amino acid sequence selected from the group consisting of:

(SEQ ID NO: 6)
VDTYDGGISVVYGLR,
(SEQ ID NO: 7)
VDTYDGDISVVYGLR,
(SEQ ID NO: 8)
DTYDGDISVVYGLR,
(SEQ ID NO: 9)
TYDGDISVVYGLRS,
(SEQ ID NO: 10)
TYDGDISVVYGLR,
(SEQ ID NO: 11)
YDGDISVVYGLRS,
(SEQ ID NO: 12)
YDGDISVVYGLR,
(SEQ ID NO: 13)
DGDISVVYGLRS,
(SEQ ID NO: 14)
DGDISVVYGLR,
(SEQ ID NO: 15)
GDISVVYGLRS,
(SEQ ID NO: 16)
GDISVVYGLR,
(SEQ ID NO: 17)
DISVVYGLRS,
(SEQ ID NO: 18)
DISVVYGLR,
(SEQ ID NO: 19)
VDVPNGDISLAYGLR,
(SEQ ID NO: 20)
DVPNGDISLAYGLRS,
(SEQ ID NO: 21)
DVPNGDISLAYGLR,
(SEQ ID NO: 22)
VPNGDISLAYGLRS,
(SEQ ID NO: 23)
VPNGDISLAYGLR,
(SEQ ID NO: 24)
PNGDISLAYGLRS,
(SEQ ID NO: 25)
PNGDISLAYGLR,
(SEQ ID NO: 26)
NGDISLAYGLRS,
(SEQ ID NO: 27)
NGDISLAYGLR,
(SEQ ID NO: 28)
GDISLAYGLRS,
(SEQ ID NO: 29)
GDISLAYGLR,
(SEQ ID NO: 30)
DISLAYGLRS,
(SEQ ID NO: 31)
DISLAYGLR,
(SEQ ID NO: 32)
VDTYDGDGSVVYGLR,
(SEQ ID NO: 33)
VDVPEGDISLAYGLR.
(SEQ ID NO: 110)
AEIDSIELSYGIK,
(SEQ ID NO: 111)
KPLAEIDSIELSYGIK,
(SEQ ID NO: 112)
KCLAECDSIELSYGIK (Cyclic),
(SEQ ID NO: 113)
KPLAEDISIELSYGIK,
(SEQ ID NO: 114)
KPLAEISDIELSYGIK,
(SEQ ID NO: 115)
KPLAEIGDIELSYGIK,
(SEQ ID NO: 116)
KPLAEGDIELSYGIK,
(SEQ ID NO: 117)
KPLAEIELSYGIK,
(SEQ ID NO: 118)
KPLAEIDSIELTYGIK,
(SEQ ID NO: 119)
KPLAEIDGIELSYGIK,
(SEQ ID NO: 120)
KPLAEIDGIELTYGIK,
(SEQ ID NO: 121)
KPLAEIGSIELSYGIK,
(SEQ ID NO: 122)
KGLAEIDSIELSYGIK,
(SEQ ID NO: 123)
KPLAGIDSIGLSYGIK,
(SEQ ID NO: 124)
Cyclic KCLAEIDSCELSYGIK,
(SEQ ID NO: 125)
LAEIDSIELSYGIK,
(SEQ ID NO: 126)
EIDSIELSYGIK,
(SEQ ID NO: 127)
IDSIELSYGIK,
(SEQ ID NO: 128)
DSIELSYGIK,
(SEQ ID NO: 129)
SIELSYGIK,
and
(SEQ ID NO: 109)
IELSYGIK.

12. The agent according to item 1, wherein the peptide comprises or consists of an amino acid sequence VDTYDGGISVVYGLR (SEQ ID NO: 6).
13. The agent according to item 1, wherein the peptide comprises or consists of an amino acid sequence VDTYDGDISVVYGLR (SEQ ID NO: 7).
14. The agent according to item 1, wherein the peptide comprises or consists of an amino acid sequence AEIDSIELSYGIK (SEQ ID NO: 110).
15. The agent according to any one of the preceding items, wherein the peptide comprises no more than 85, such as no more than 80, such as no more than 75, such as no more than 70, such as no more than 65, such as no more than 60, such as nor more than 55, such as no more than 50, such as no more than 55, such as no more than 40 amino acids, such as no more than 35, such as no more than 30, such as no more than 28, such as no more than 26, such as no more than 24, such as no more than 22, such as no more than 20, such as no more than 19, such as no more than 18, such as no more than 17, such as no more than 16, such as no more than 15, such as no more than 14, such as no more than 13, such as no more than 12, such as no more than 11, such as no more than 10 amino acids.
16. The agent according to any one of the preceding items, wherein the peptide comprises at least 2 additional amino acids, such as at least 3, such as at least 4, such as at least 5, such as at least 6, such as at least 7, such as at least 8, such as at least 9, such as at least 10, such as at least 15 or such as at least 20 amino acids conjugated to the N- or C-terminus of the peptide.
17. The agent according to any one of the preceding items, wherein the agent is non-naturally occurring.
18. The agent according to any one of the preceding items, wherein the agent is conjugated to a moiety.
19. The agent according to item 15, wherein the moiety is selected from the group consisting of polyethylene glycol (PEG), monosaccharides, fluorophores, chromophores, radioactive compounds, and cell-penetrating peptides.
20. The agent according to any one of the preceding items, wherein the agent is further modified such as by glycosylation, PEGylation, amidation, esterification, acylation, acetylation and/or alkylation.
21. The agent according to any one of the preceding items, wherein the agent comprises or consists of a tandem repeat comprising two or more repeat units.
22. The agent according to item 21, wherein the repeat unit comprises or consists of the amino acid sequence of any one or more of the sequences as described in the preceding items.
23. The agent according to any of the preceding items, wherein the agent is fused to another polypeptide.
24. The agent according to item 20, wherein the said polypeptide is selected from the group consisting of glutathione-S-transferase (GST) and protein A.
25. The agent according to any of the preceding items, wherein the agent is fused to a tag.
26. The agent according to item 25, wherein the tag is an oligo-histidine tag.
27. The agent according to any of the preceding items, wherein the agent is cyclic.
28. The agent according to any of the preceding items, wherein the agent is capable of forming at least one intramolecular cysteine bridge.
29. The agent according to any one of the preceding items, wherein the agent is a variant of the peptide, wherein the variant comprises or consists of a sequence wherein any one amino acid has been altered for another proteinogenic or non-proteinogenic amino acid, with the proviso that no more than five amino acids are so altered.
30. The agent according to item 29, wherein the variant comprises or consists of a sequence wherein no more than five amino acids are altered for another proteinogenic or non-proteinogenic amino acid, such as no more than 4 amino acids, such as no more than 3 amino acids, such as no more than 2 amino acids, such as no more than 1 amino acid is altered.
31. The agent according to any one of the preceding items, wherein one or more amino acids are conservatively substituted.
32. The agent according to any one of the preceding items, wherein the peptide comprises or consists of one or more additional amino acids, inserted at the N- and/or C-terminus and/or internally within the sequence.
33. The agent according to any one of the preceding items, wherein the peptide has one additional amino acid.
34. The agent according to any of the preceding items, wherein the agent further comprises a detectable moiety.
35. The agent according to item 34, wherein the detectable moiety comprises or consists of a radioisotope.
36. The agent according to item 35, wherein the radioisotope is selected from the group consisting of ^{99m}Tc, ¹¹¹In, ⁶⁷Ga, ⁶⁸Ga, ⁷²As, ⁸⁹Zr, ¹²³I and ²⁰¹Tl.
37. The agent according to item 34, wherein the detectable moiety is detectable by an imaging technique such as SPECT, PET, MRI, optical or ultrasound imaging.
38. A composition comprising the agent according to any one of the preceding items.
39. The composition according to item 38, wherein the composition is a pharmaceutical composition.
40. The composition according to item 38, wherein the composition is a cosmetic composition.
41. The composition according to any one of the preceding items, wherein the composition is a coating.
42. An implant comprising an agent according to any one of the preceding items.
43. The implant according to item 42, wherein the implant is coated with a composition comprising an agent according to any one of the preceding items.
44. The implant according to item 42, wherein the implant is of a biomaterial.
45. The implant according to item 44, wherein the biomaterial is bone.
46. The implant according to item 42, wherein the implant is a medical device.
47. The implant according to item 46, wherein the medical device is a stent.
48. The agent according to any one of the preceding items for use in increasing angiogenesis in a subject.
49. The agent according to any one of the preceding items for use as an angiogenesis inducer.
50. The agent according to any one of the preceding items for use in improving myocyte survival in cardiovascular disease.
51. The agent according to any one of the preceding items for use in improving neural cell survival in cerebrovascular disease.
52. The agent according to any one of the preceding items for use in the treatment of a disease or disorder associated with reduced or impaired angiogenesis.
53. A method for inducing vascularization, said method comprising administering the agent according to any one of the preceding items to a subject.
54. The agent according to any one of the preceding items for use in the treatment or prevention of a disease or disorder in a subject, wherein the disease or disorder is selected from the group consisting of:
   i. a disease of the circulatory system,
   ii. an injury of external cause,
   iii. a disease of the immune system,
   iv. a disease of the nervous system, and
   v. a disease of the musculoskeletal system or connective tissue.
55. The agent for use according to any one of the preceding items, for use in the treatment or prevention of a complication associated with a disease or disorder selected from the group consisting of:
   i. a disease of the circulatory system,
   ii. an injury of external cause,
   iii. a disease of the immune system,
   iv. a symptom, sign, or clinical finding of the circulatory system,
   v. a disease of the myocardium or cardiac chambers, and
   vi. an endocrine, nutritional or metabolic disease.
56. The agent for use according to any one of the preceding items, wherein the disease of the circulatory system is selected from the group consisting of:
   i. an ischaemic heart disease,
   ii. a cerebrovascular disease,
   iii. a disease of coronary artery,
   iv. a disease of the arteries, arterioles, or capillaries,
   v. a symptom, sign, or clinical finding of the circulatory system,
   vi. a disease of the myocardium or cardiac chambers, and
   vii. an endocrine, nutritional or metabolic disease.
57. The agent for use according to any one of the preceding items, wherein the ischaemic heart disease is selected from the group consisting of:
   i. acute ischaemic heart disease, such as myocardial infarction, such as acute myocardial infarction and unspecified acute ischaemic heart disease (acute coronary syndrome),
   ii. chronic ischaemic heart disease, such as other specific chronic ischaemic heart disease (cardiovascular arteriosclerosis),
   iii. angina pectoris, such as unstable angina pectoris, and
   iv. obstructive arteriosclerosis.
58. The agent for use according to any one of the preceding items, wherein the myocardial infarction is ST elevation myocardial infarction (STEMI) or non-ST elevation myocardial infarction (NSTEMI).
59. The agent for use according to any one of the preceding items, wherein the STEMI or NSTEMI presents with subsequent certain current complications, such as within a 28 day period.
60. The agent for use according to any one of the preceding items, wherein the disease of the circulatory system is cardiovascular sclerosis.
61. The agent for use according to any one of the preceding items, wherein the disease of the circulatory system is systemic sclerosis or associated with systemic sclerosis.
62. The agent for use according to any one of the preceding items, wherein the cerebrovascular disease is selected from the group consisting of:
   i. cerebral ischaemia, such as cerebral ischaemic stroke (stroke), such as cerebral infarction, and
   ii. asymptomatic stenosis of intracranial or extracranial artery (cerebral arteriosclerosis).
63. The agent for use according to any one of the preceding items, wherein the cerebral ischaemic stroke is associated with a patient history of transient ischaemic attack (TIA) or cerebral infarction without residual deficits.
64. The agent for use according to any one of the preceding items, wherein the cerebral ischaemic stroke is associated with a patient history of traumatic brain injury or sequelae of cerebrovascular disease.
65. The agent for use according to any one of the preceding items, wherein the cerebrovascular disease is selected from the group consisting of:
   x. nontraumatic subarachnoid haemorrhage,
   xi. nontraumatic intracerebral haemorrhage,
   xii. other and unspecified nontraumatic intracranial haemorrhage,
   xiii. cerebral infarction,
   xiv. occlusion and stenosis of precerebral arteries not resulting in cerebral infarction,
   xv. occlusion and stenosis of cerebral arteries, not resulting in cerebral infarction,
   xvi. other cerebrovascular diseases,
   xvii. cerebrovascular disorder associated with other diseases (cerebrovascular disorders classified elsewhere), and
   xviii. sequelae of cerebrovascular disease.
66. The agent for use according to any one of the preceding items, wherein the disease of the arteries, arterioles, or capillaries is selected from the group consisting of:
   i. atherosclerosis,
   ii. aortic aneurysm and dissection,
   iii. an other aneurysm,
   iv. an other peripheral vascular disease,
   v. arterial embolism and thrombosis,
   vi. atheroembolism,
   vii. septic arterial embolism,
   viii. an other disorder of arteries and arterioles,
   ix. a disease of the capillaries, and
   x. a disorder of the arteries, arterioles, or capillaries associated with another disease (a disorder of the arteries, arterioles, or capillaries in a disease classified elsewhere).
67. The agent for use according to any one of the preceding items, wherein the disease of the arteries, arterioles, or capillaries is chronic arterial occlusive disease, such as atherosclerotic chronic arterial occlusive disease or vascular sclerosis.
68. The agent for use according to any one of the preceding items, wherein the symptom, sign, or clinical finding of the circulatory system is a symptom or sign involving the circulatory system, such as an abnormal blood-pressure reading without diagnosis, such as cardiac arrest.
69. The agent for use according to any one of the preceding items, wherein the disease of the myocardium or cardiac chambers is cardiomyopathy, such as dilated cardiomyopathy.
70. The agent for use according to any one of the preceding items, wherein the disease of the immune system is non-organ specific systemic autoimmune disorder, such as vasculitis, such as thromboangiitis obliterans (Buerger's disease).
71. The agent for use according to any one of the preceding items, wherein the disease of the nervous system is selected from the group consisting of:
   i. a movement disorder, such as parkinsonism, such as Parkinson's disease,
   ii. multiple sclerosis or other white matter disorders, such as multiple sclerosis, and
   iii. disorders with neurocognitive impairment as a major feature, such as Alzheimer's disease.
72. The agent for use according to any one of the preceding items, wherein the disease of the musculoskeletal system or connective tissue is a condition associated with the spine, such as herniated disc.
73. The agent for use according to any one of the preceding items, wherein the disease of the circulatory system, the disease of the immune system, the disease of the nervous system, or the disease of the musculoskeletal system or connective tissue is or is associated with diabetes mellitus.
74. The agent for use according to any one of the preceding items, wherein the diabetes mellitus is selected from Type 1 diabetes mellitus and Type 2 diabetes mellitus.
75. The agent for use according to any one of the preceding items, wherein the subject is suffering from diabetes mellitus.
76. The agent for use according to any one of the preceding items, wherein the subject is a mammal.
77. The agent for use according to item 76, wherein the mammal is a human.
78. A method for treating or preventing a disease or disorder in a subject, wherein the disease or disorder is selected from the group consisting of:
   i. a disease of the circulatory system,
   ii. an injury of external cause,
   iii. a disease of the immune system,
   iv. a disease of the nervous system, and
   v. a disease of the musculoskeletal system or connective tissue, the method comprising administering a therapeutically effective amount of an agent according to any one of the preceding items to an subject in need thereof.
79. Use of an agent according to any one of the preceding items for the manufacture of a medicament for the treatment or prevention of a disease or disorder selected from the group consisting of:
   i. a disease of the circulatory system,
   ii. an injury of external cause,
   iii. a disease of the immune system,
   iv. a disease of the nervous system, and
v. a disease of the musculoskeletal system or connective tissue.
80. The agent according to any one of the preceding items, for use in improving vascularisation post surgery.
81. The agent for use according to any one of the preceding items, wherein the surgery is surgery of the cardiovascular system.
82. The agent for use according to any one of the preceding items, wherein the surgery of the cardiovascular system is a vascular graft.
83. The agent according to any one of the preceding items for use in increasing angiogenesis in a subject having an implant.
84. The agent for use according to any one of the preceding items, wherein the implant is of a biomaterial.
85. The agent for use according to any one of the preceding items, wherein the biomaterial is bone.
86. The agent for use according to any one of the preceding items, wherein the implant is a medical device.
87. The agent for use according to any one of the preceding items, wherein the medical device is a stent.
88. The agent according to any one of the preceding items for use in increasing angiogenesis in a subject undergoing or having undergone transplant.
89. The agent for use according to any one of the preceding items, wherein the transplant is of an organ, of a tissue, or of a cell.
90. The agent for use according to any one of the preceding items, wherein the transplant of a cell is of bone marrow cells.
91. The agent for use according to any one of the preceding items, wherein the transplant of an organ is of a heart or a cardiovascular tissue.

### EXAMPLES

### Example 1: Polypeptides of the Disclosure Stimulates Endothelial and Vascular Smooth Muscle Cell Proliferation

### Methods

Cell proliferation was evaluated with BrdU Cell Proliferation Elisa Kit (Abcam, UK). After incubation at 37° C. overnight with respective complete medium, for NRP-1 knock down-relative experiments, HUVECs and HCASMCs on 96-well plates were transfected with siRNA/siRNA-NC directly; for test polypeptide stimulations, HUVECs and HCASMCs were starved with low-serum condition for additional 24 hours and 48 hours before treatments. At 32 hours after transfection or polypeptide stimulations, cells were cultured with BrdU reagent for another 16 hours to determine cell proliferation. Subsequent steps were performed as manufacturer's instructions and data were read by Wallac 1420 Victor 2 (Perkin Elmer, USA).

For stimulation of endothelial cell proliferation, human umbilical cord endothelial cells were first treated with a scramble non-coding siRNA (NC) or siRNA for NRP-1, subsequently they were treated with FOL26 in concentrations of 10 nM, 100 nM, and 1000 nM, and DNA synthesis analyzed by measuring the uptake of BrdU.

### Results

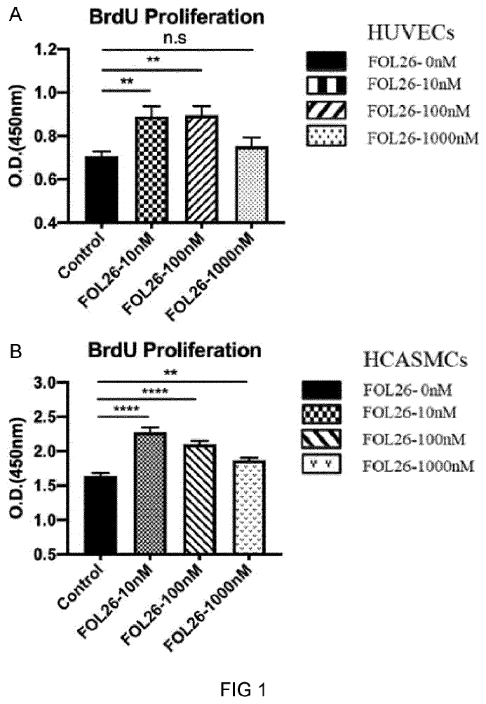

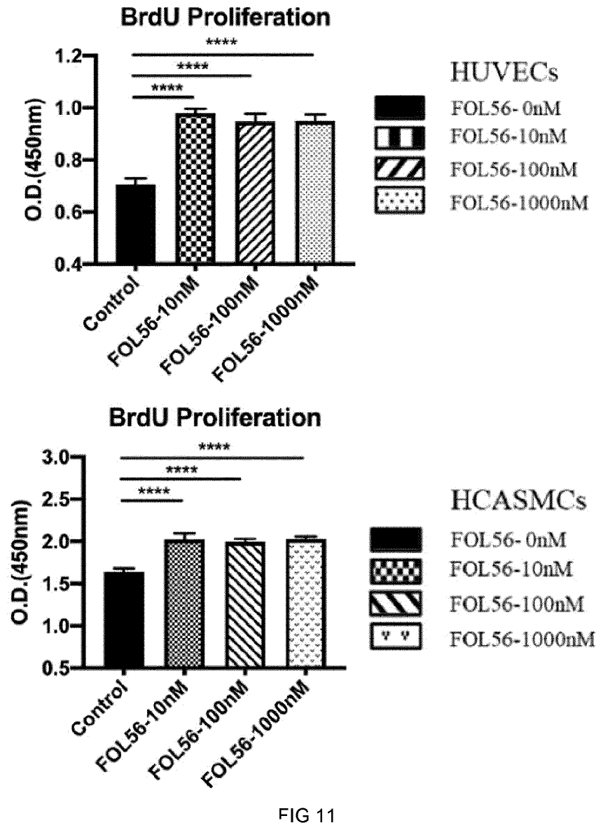

The results are shown in FIG. 1 for FOL26 and FIG. 11 for FOL56. Both test polypeptides induced a significant increase in proliferation of the HUVECs and HCASMCs.

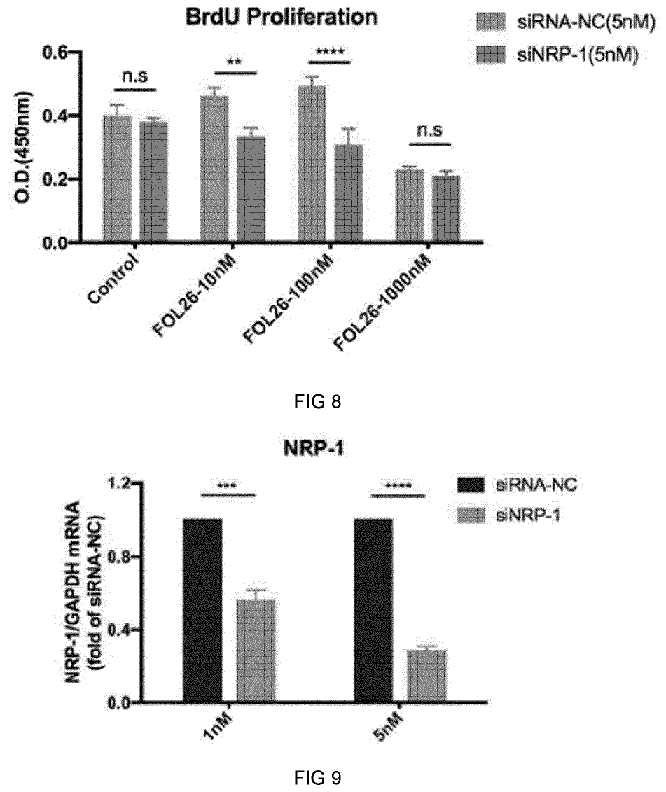

FOL26 stimulation of endothelial cell proliferation is NRP-1 dependent (FIG. 8).

### Conclusion

The polypeptides of the disclosure stimulates endothelial and vascular smooth muscle cell proliferation.

### Example 2: Polypeptides of the Disclosure Induces Tube Formation in HUVECs

### Methods

Cells were transfected for 48 hours or performed with 0.5% supplement starvation for 24 hours on 6-well plates as described above and dissociated by Accutase (Gibco, USA) to obtain cell suspension. Geltrex reduced growth factor basement membrane matrix ((Invitrogen, CA, USA) was thawed at 4° C. overnight and added into a 96-well plate (50 pl/well) next. The coated 96-well plate was placed in the incubator more than 30 mins for further use and the cell suspension was seeded into it at a density of 1.5 x 104/well. For NRP-1 silencing experiments, cells were cultured with complete medium. For polypeptide treatment experiments, cells were stimulated with complete medium plus the different concentration of polypeptide. For NRP-1 rescued experiments, cells transfected with siRNA were cultured with complete medium and NRP-1 stimulations. After a 24-hour incubation, images were recorded by an inverted microscope (Nikon, Eclipse TE2000-U, Japan) and the sum length of master segments of vessels in each image were quantified using Angiogenesis Analyzer plug-in ImageJ software program (pixel/unit). Data were analyzed with photographs from 8-14 wells per condition.

Human umbilical cord endothelial cells were first treated with a scramble non-coding siRNA (NC) or siRNA for NRP-1, subsequently treated with FOL26 in concentrations of 10 nM, 100 nM, and 1000 nM, and endothelial tube formation and the total master segemnts were measured using ImageJ. gene expression of PECAM-1 (CD31) analyzed.

### Results

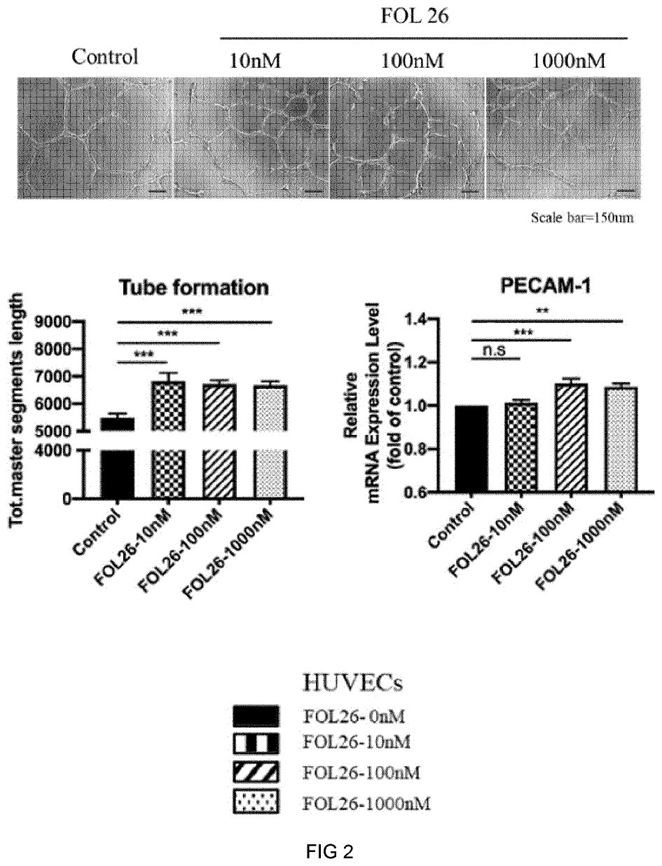

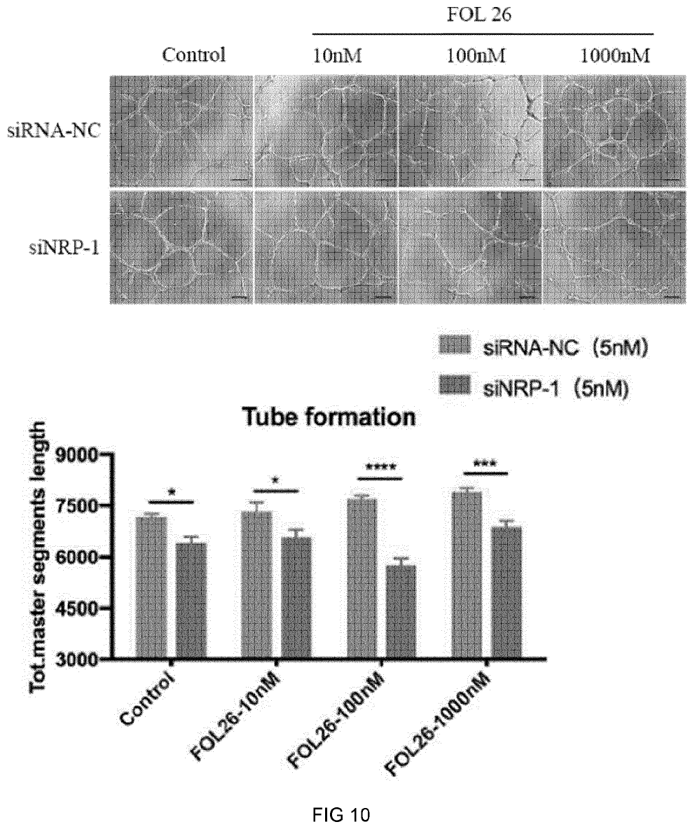

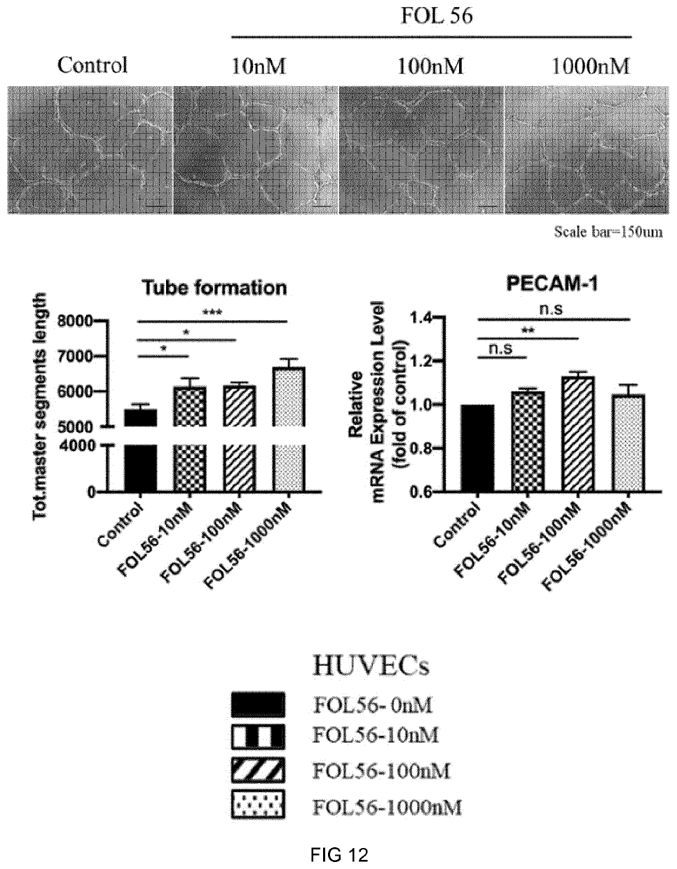

The results are shown in FIG. 2 for FOL26 and in FIG. 12 for FOL56. The test polypeptides significantly increased tube formation in the HUVECs. The polypeptides also increased expression of PECAM-1. FIG. 10 shows that FOL26 increased tube formation compared to control in a NRP-1 dependent way.

### Conclusion

Polypeptides of the disclosure induces tube formation in HUVECs.

### Example 4: Polypeptides of the Disclosure Prevents Apoptosis in HUVECs and HCASMCs

### Methods

Human Umbilical Vein Endothelial Cells (HUVECs) and Human Coronary Artery Smooth Muscle Cells (HCASMCs) were purchased from Thermo Fisher. Cells were grown in Medium 200 containing 2% Low Serum Growth Supplement and Medium 231 with 5% Smooth Muscle Growth Supplement (Invitrogen, CA, USA) respectively with addition of Antibiotic-Antimycotic (1%, Invitrogen, CA, USA). Test polypeptide and sFasL (PeproTech, NJ, USA) were dissolved in the respective complete medium.

Caspase-Glo 3/7 Assay (Promega, USA) was performed to evaluate cell apoptosis. HUVECs and HCASMCs were seeded into 96-well plates at a density of 3-5×103 per well. For additional NRP-1 on cells transfected siRNA, NRP-1 was added 24 hours after transfection and the mixture of Caspase-Glo 3/7 was added 48 hours after polypeptide stimulations. Subsequent steps were performed as manufacturer's instructions using the FOL26 and FOL56 as test compounds. Data were read by Wallac 1420 Victor 2 (Perkin Elmer, USA).

### Results

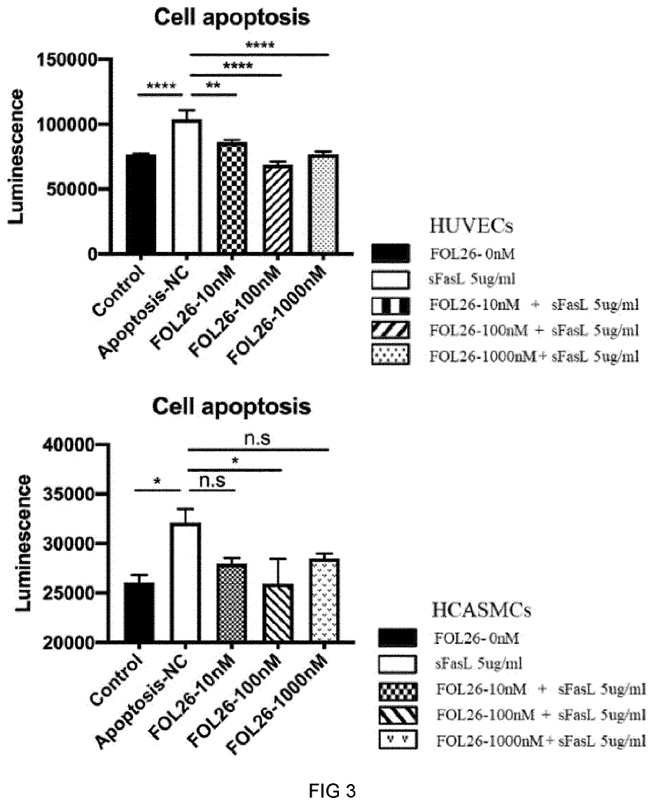

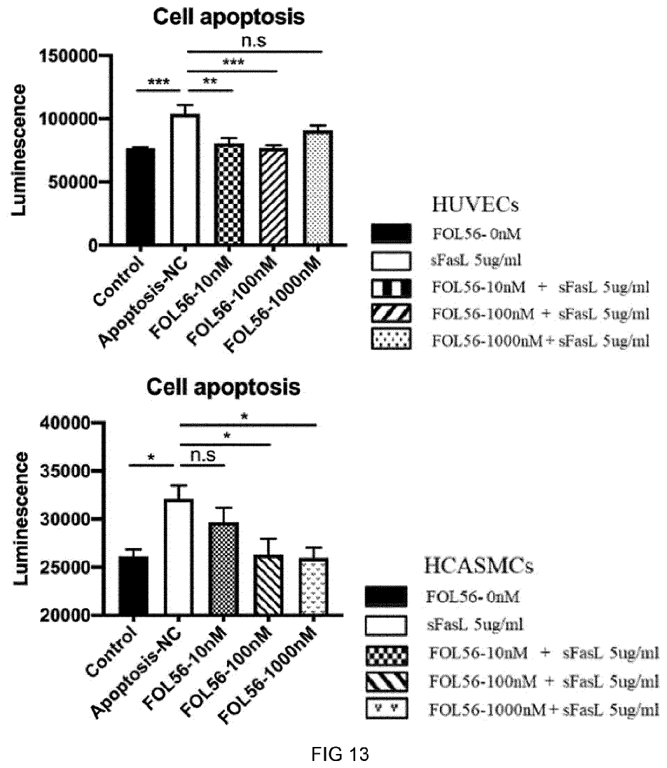

The results are shown in FIG. 3 for FOL26 and FIG. 13 for FOL56 on both HUVECs and HCASMCs. Both polypeptides reduced sFasL-induced apoptosis in the cell lines.

### Conclusion

Polypeptides of the disclosure prevents apoptosis in HUVECs and HCASMCs.

### Example 5: Effect on Gene Expression of Polypeptides of the Disclosure in HUVECs and HCASMCs

### Methods

Cells on 6-well plates were washed with cold DPBS (Gibco, USA) and homogenized in Trizol reagent (Invitrogen, CA, USA). Total RNA was extracted using PureLink RNA Mini Kit (Invitrogen, CA, USA), followed by nucleic acid quantification with NanoDrop 2000c (Thermo Fisher Scientific, USA). To determine mRNA expression of target genes, real-time qPCR was performed using by KAPA SYBR FAST One-Step qRT-PCR Master Mix Kit (KAPA Biosystems, USA) on LightCycler480 system (Roche, Mannheim, Germany). All primers were purchased from miScript Primer Assays (Qiagen, Valencia, CA) and GAPDH mRNA expression of each sample was used for normalization. Cells on 6-well plates were washed with cold DPBS and lysed with the mixture of lysis buffer and protease inhibitors on ice, then centrifuged at 15,000 g for 20 min at 4° C. Supernatant was performed to measure the protein concentration using BCA Protein Assay Kit (Thermo Fisher Scientific, USA). Protein sample was loaded onto SDS-PAGE gels and separated with electrophoresis, followed by transferred to PVDF membranes (Millipore, USA). Membranes were blocked with 5% nonfat milk. Primary antibodies (Abcam, UK) and secondary antibodies were performed to incubate with membranes at 4° C. overnight and at room temperature for 1 hour respectively.

### Results

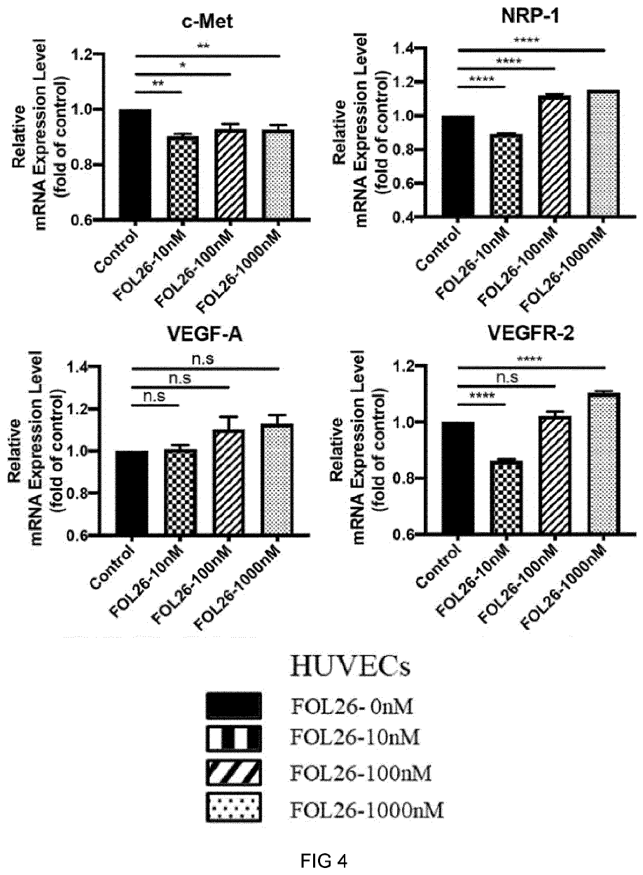

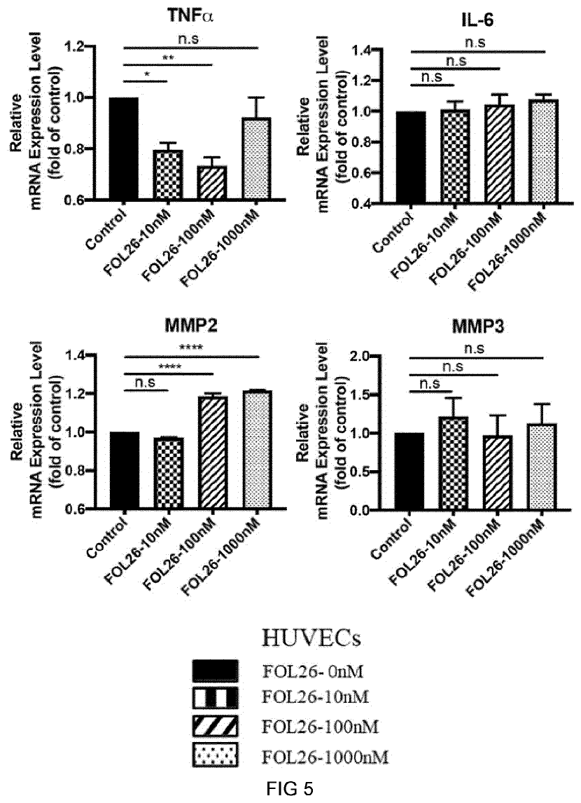

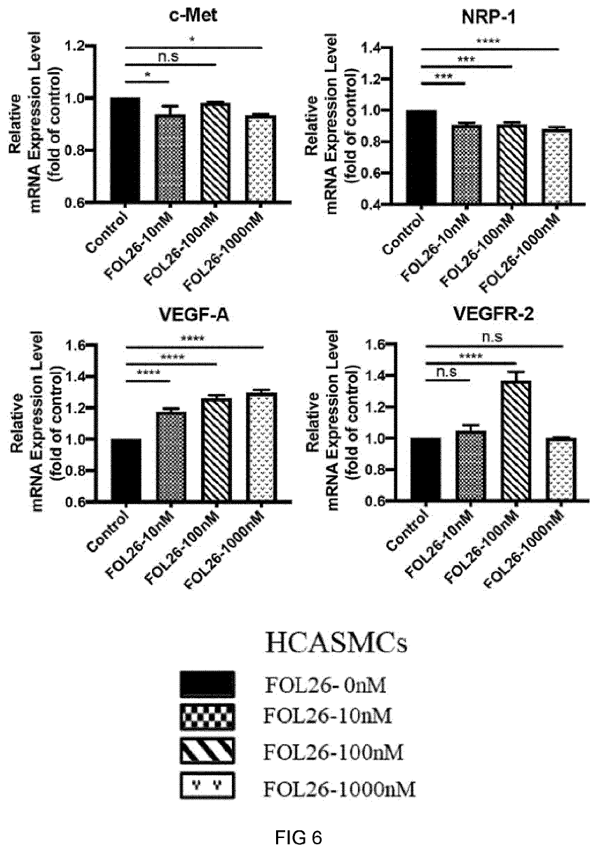

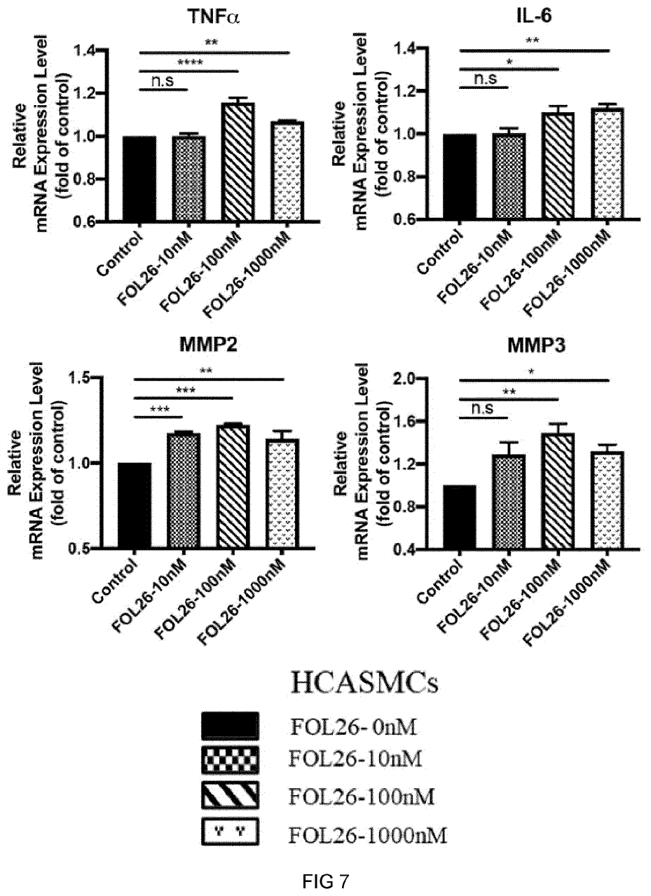

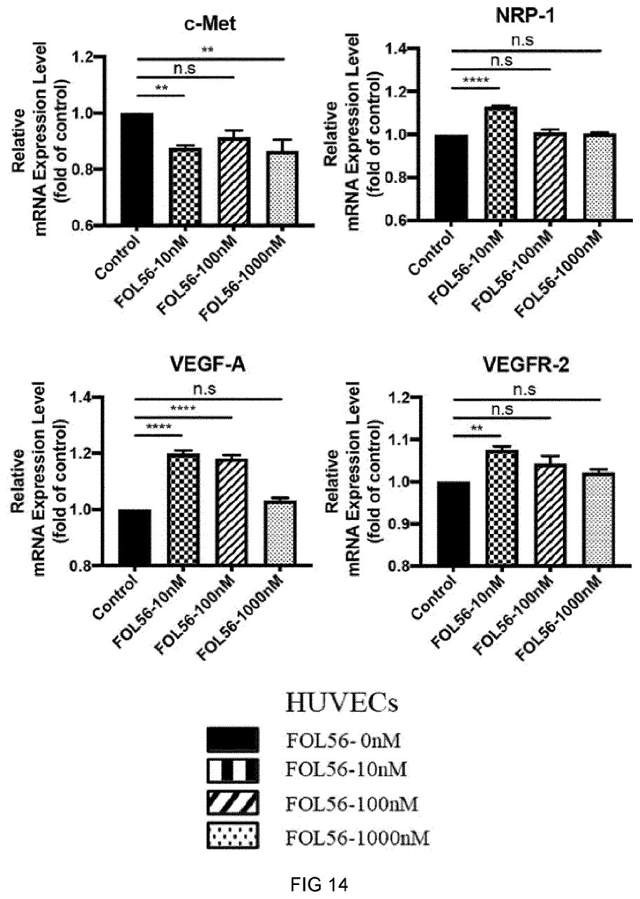

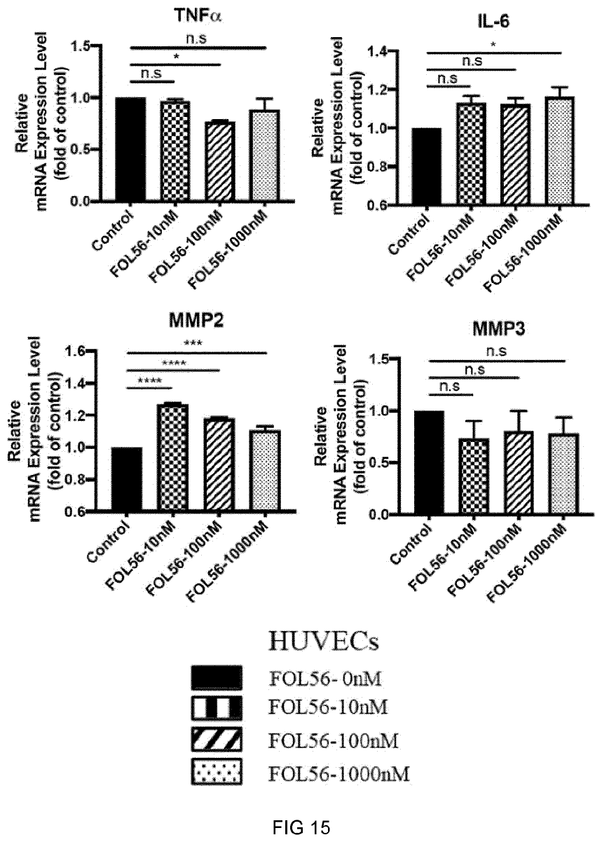

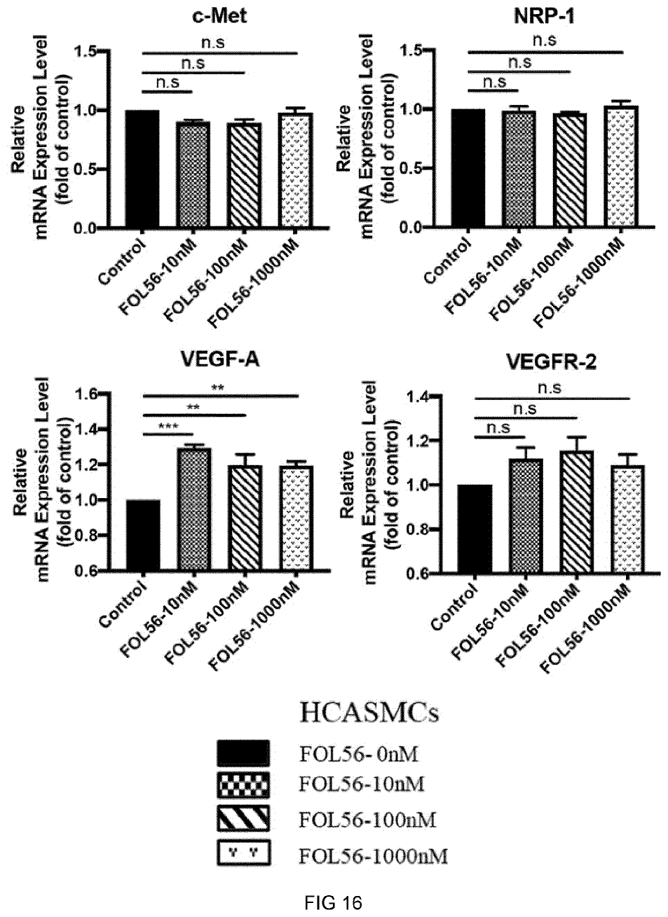

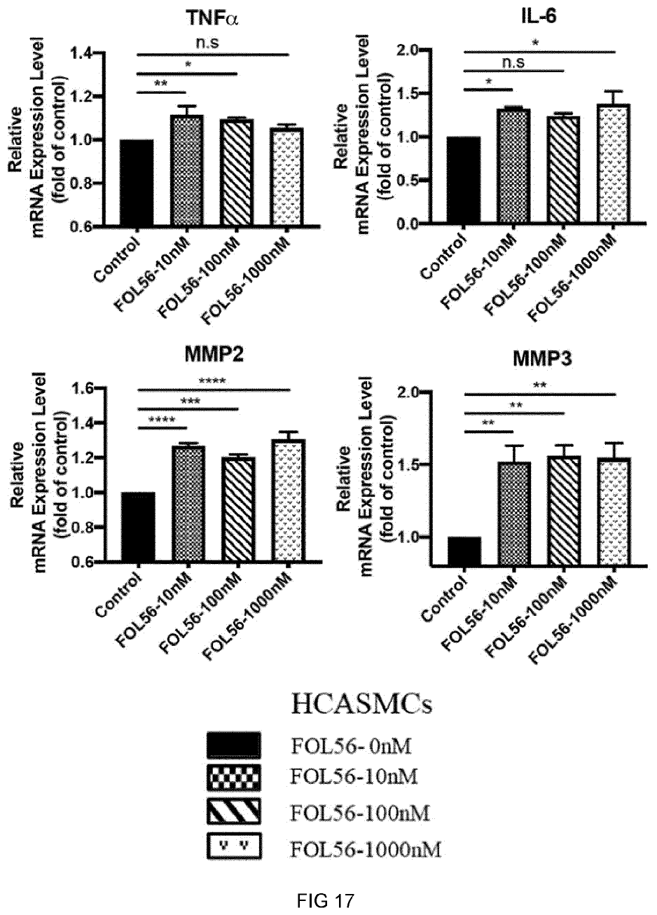

The results are shown in FIGS. 4-7 for FOL26 and FIGS. 14-17 for FOL56. Table 1 shows various important biological mediators in tissue regenerative processes.

TABLE 1
Growth factors and cytokines affecting various
steps in regeneration of tissues.

| | |
|---|---|
| Monocyte chemotaxis | PDGF, FGF, TGF-β |
| Fibroblast migration | PDGF, EGF, FGF, TGF-β, TNF, IL-1 |
| Fibroblast proliferation | PDGF, EGF, FGF, TNF |
| Angiogenesis | VEGF, Ang, FGF |
| Collagen synthesis | TGF-β, PDGF |
| Collagenase secretion | PDGF, EGF, FGF, TNF, TGF-β inhibits |

### Conclusion

Polypeptides of the disclosure induces a milieu inducing tissue regeneration by TNF and IL-6 expression likely to promote fibroblast migration and proliferation, collagen secretion angiogenesis, and induce correct tissue regeneration by inducing cell remodelling through MMP expression.

### Example 6. Effect of FOL-026 Peptide on Endothelial Cell Function and Gene Expression

### Method

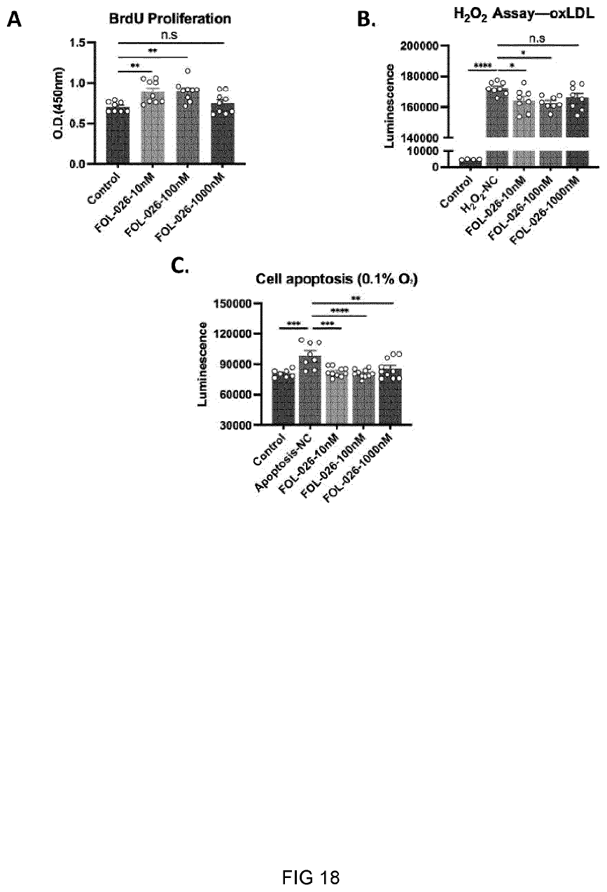

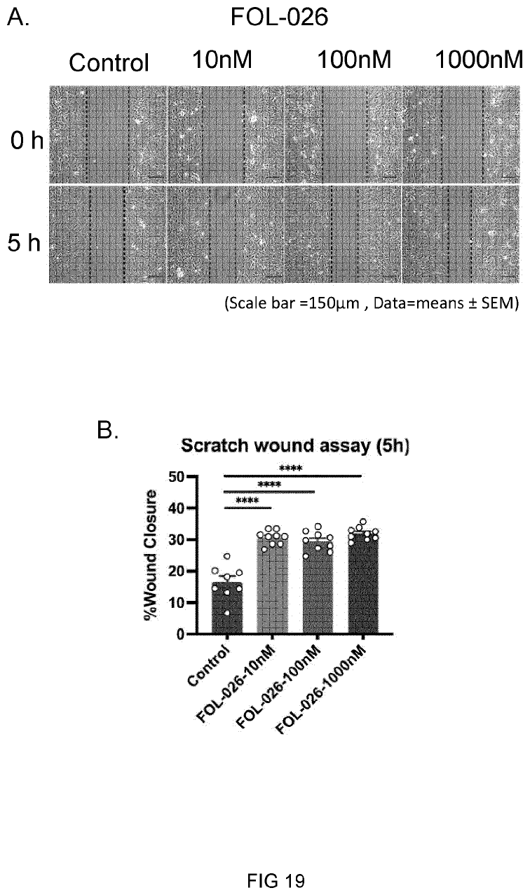

The impact of FOL-026 peptide on human umbilical vascular endothelial cells (HUVECs) proliferation (FIG. 18a) and apoptosis (FIG. 18c) induced with low-oxygen condition was determined by BrdU uptake (n=8-9 per group) and active caspase-3/-7 (n=7-10 per group) separately. The ROS level (FIG. 18b) activated by 50 ug/ml oxidized Low-density Lipoprotein (oxLDL) for 2 hours in cells pre-incubated with FOL-026 peptides was assessed using H2O2 measurement (n=4-8 per group). Representative pictures of endothelial migration into an in vitro scratch injury were shown (FIG. 19a) and the wound closure rate (FIG. 19b) in HUVECs stimulated with increasing concentration of FOL-026 peptide was quantified by image J software (n=8-9 per group). Scale bar=150 um. Data are presented as means±SEM and acquired from 3 to 4 independent replicate tests. *P<0.05, **P<0.01, ****P<0.0001 by one-way ANOVA and Dunnett's post-hoc test.

### Results

The results shown in FIG. 18a indicates that the proliferation of on human umbilical vascular endothelial cells increases with the addition of the FOL-026 peptide. FOL-026 also alleviates the effect of low oxygen induced apoptosis in HUVECs and can reduce the amount of ROS levels induced by oxLDL (FIGS. 18c and 18b).

FIGS. 19a and 19b shows that addition of FOL-026 accelerates HUVEC wound closure.

### Conclusions

The FOL-026 peptide has a stimulatory effect on the proliferation of endothelial cells.

### Example 7. Effect of FOL-026 Peptide on Angiogenesis In Vitro and Vivo

### Method

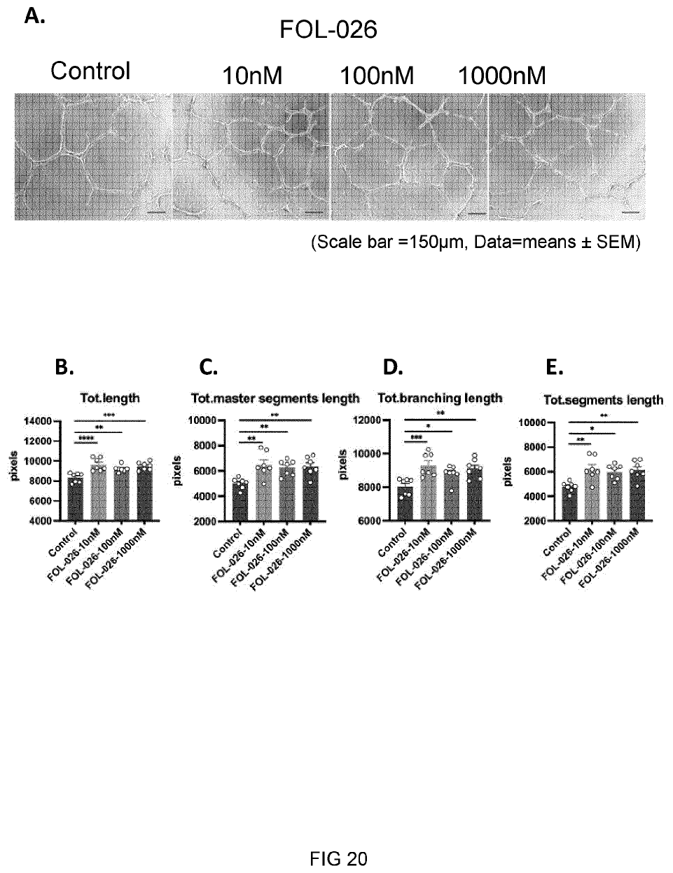

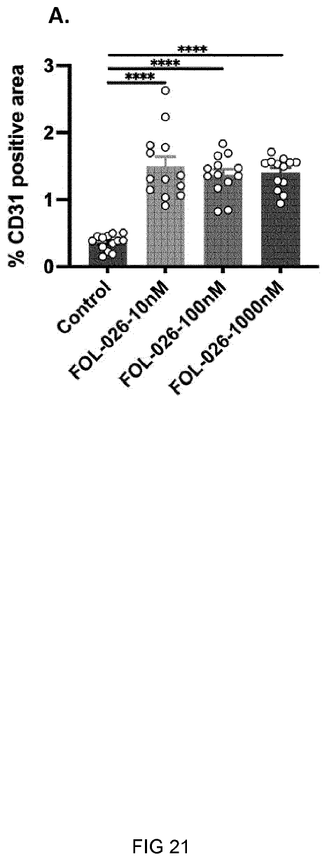

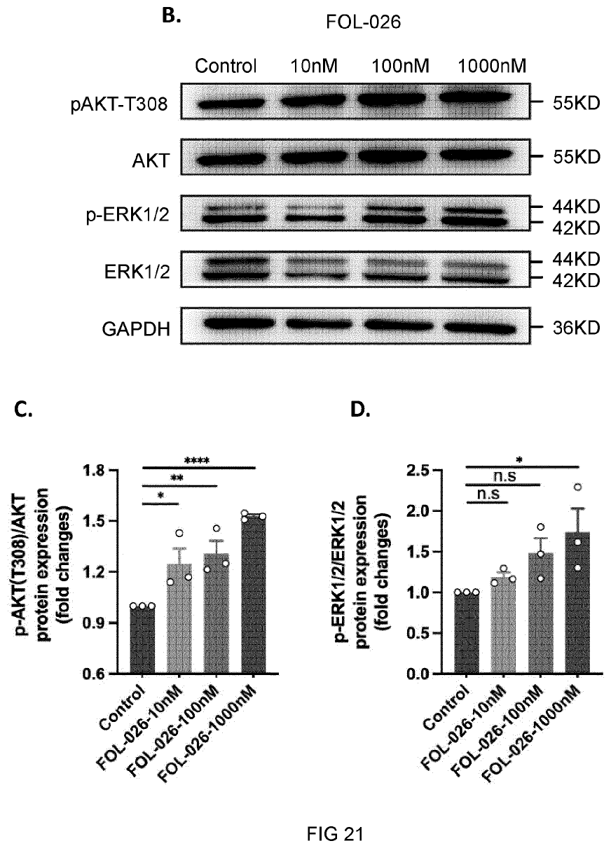

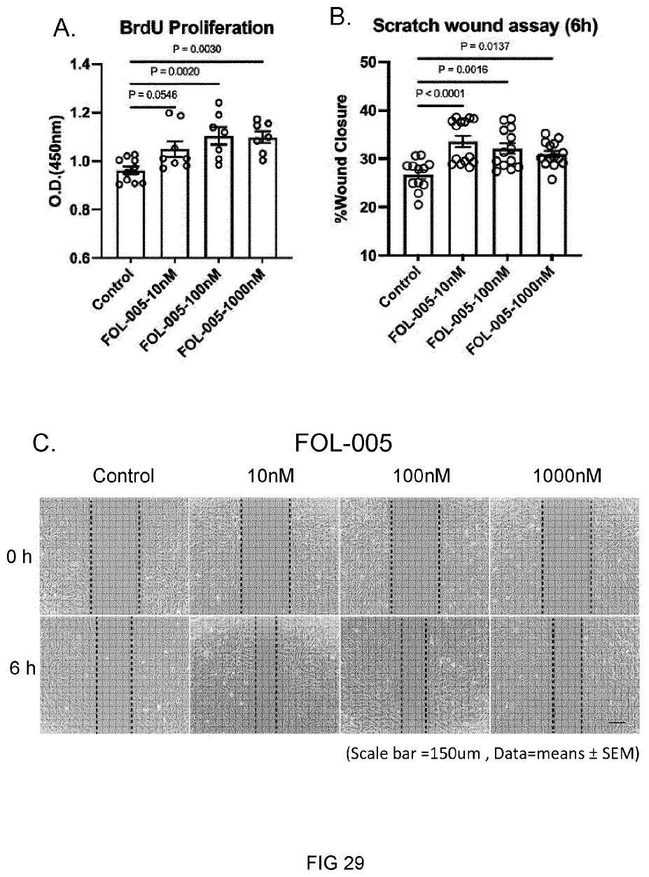

Representative images of tube formation in HUVECs treated with FOL-026 peptides are shown in FIG. 20a. Quantified measurement of total length (FIG. 20b), total master segments length (FIG. 20c), total branching length (FIG. 20d) and total segments length (FIG. 20e) were performed to evaluate angiogenesis in vitro (n=7 per group for FIG. 29 b-e). The quantification of vessel density was assessed with CD31+ positive area (FIG. 21a, n=13 per group). Western blot analysis of phosphorylated AKT (pAKT-T308), AKT, phosphorylated ERK1/2 (p-ERK1/2) and ERK1/2 in endothelial cells stimulated with FOL-26 peptides for 48 hours (FIG. 21b). Quantification of phosphorylation intensities normalized to respective total expressions (FIG. 21c-d, n=3 per group).

Data are presented as means±SEM and acquired from 3 to 4 independent replicate tests. *P<0.05, **P<0.01, ***P<0.001, ****P<0.0001 by one-way ANOVA and Dunnett's post-hoc test.

### Results

FIGS. 20 a-d shows the positive effect FOL-026 has on tube formation. It demonstrates the increased vessel density effect of FOL-026 and how FOL-026 induces upregulation of angiogenesis related genes.

### Conclusion

The FOL-026 peptide affects angiogenesis both in vitro and in vivo.

### Example 8: Effect of NRP-1 Knock-Down on Endothelial Cell Function Induced with FOL-026 Peptide

### Method

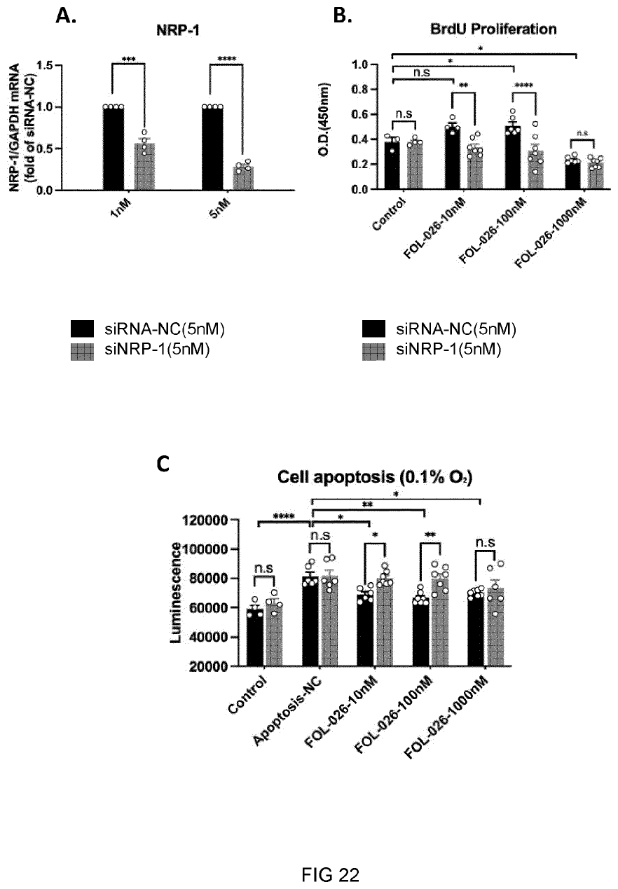

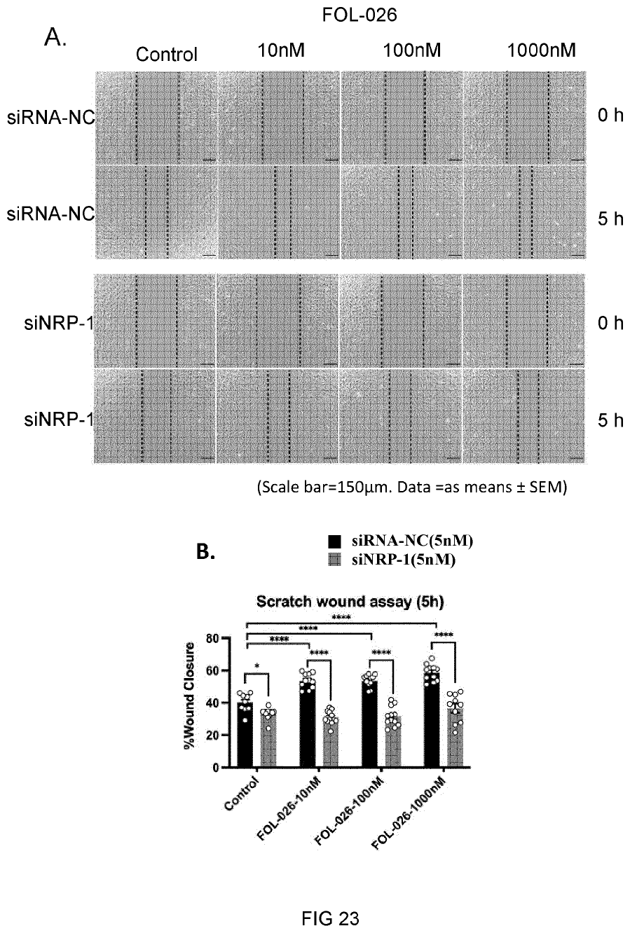

The efficiency of small interfering RNA transfection for 48 hours in HUVECs was confirmed by qRT-PCR (n=4) (FIG. 22a). Effect of FOL-026 peptide stimulation on cell proliferation (FIG. 22b) and cell apoptosis at 0.1% oxygen (FIG. 22c) in HUVECs transfected with siRNA-NC or siNRP-1 was evaluated by BrdU incorporation (n=4-7 per group) and active caspase-3/-7 ((n=4-7 per group). Representative pictures of endothelial cells at 0 and 5 hours after scratch wounding are shown in 23a. The wound closure rate (FIG. 23b) of siRNA-NC/siNRP-1-transfected HUVECs stimulated with increasing concentration of FOL-026 peptide was quantified by image J software (n=9-12 per group). Scale bar=150 um. Data are presented as means±SEM and acquired from 3 to 4 independent replicate tests. *P<0.05, **P<0.01, ***P<0.001, ****P<0.0001 by Student's t test for A, two-way ANOVA followed with Sidak's and Dunnett's multiple comparisons test for B, C and E.

### Results

FIGS. 22a-c shows that reducing the NRP-1 expression blocks the effect of FOL-026 FIGS. 23a-b show that FOL-026 has a positive effect on wound closure in siRNA-NC/siNRP-1-transfected HUVECs.

### Conclusion

FOL-026 stimulates wound closure in HUVECs by binding to NRP-1

### Example 9: Effect of NRP-1 Knock-Down on Tube Formation Induced with FOL-026 Peptide

### Method

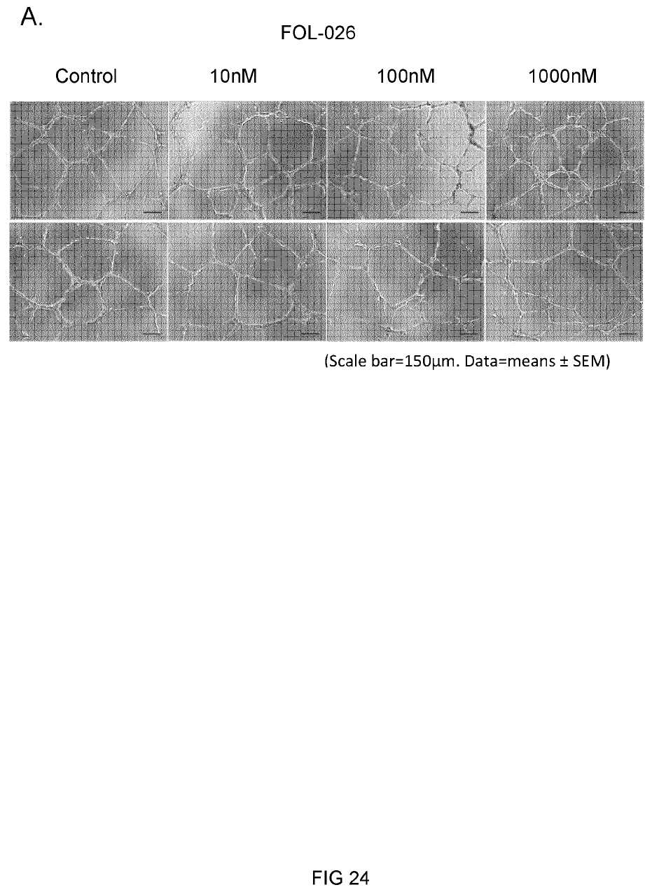

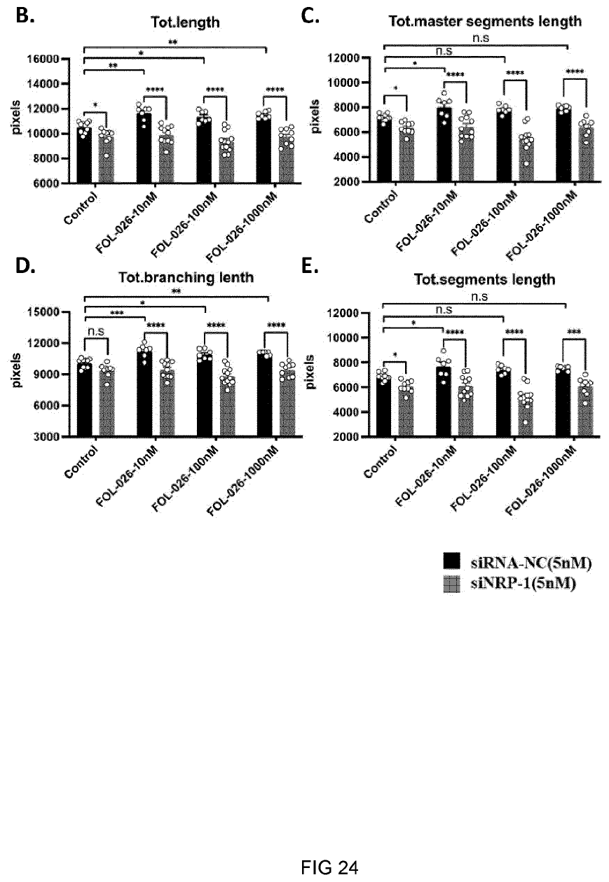

Tube formation with rising dose of FOL-026 peptide treatment in siRNA-NC or siNRP-1 group (FIG. 24a) and total length (FIG. 24b), total master segments length (FIG. 24c), total branching length (FIG. 24d) and total segments length (FIG. 24e) was calculated using Angiogenesis Analyzer plug-in ImageJ (n=7-12 per group). Scale bar=150 um. Data are presented as means±SEM and acquired from 3 to 4 independent replicate tests. *P<0.05, **P<0.01, ***P<0.001, ****P<0.0001 by two-way ANOVA followed with Sidak's and Dunnett's multiple comparisons test.

### Results and Conclusion

The FOL-026 peptide induce tube formation in siRNA-NC and siNRP-1.

### Example 10: Effect of FOL-026 Peptide on Smooth Muscle Cell Function

### Method

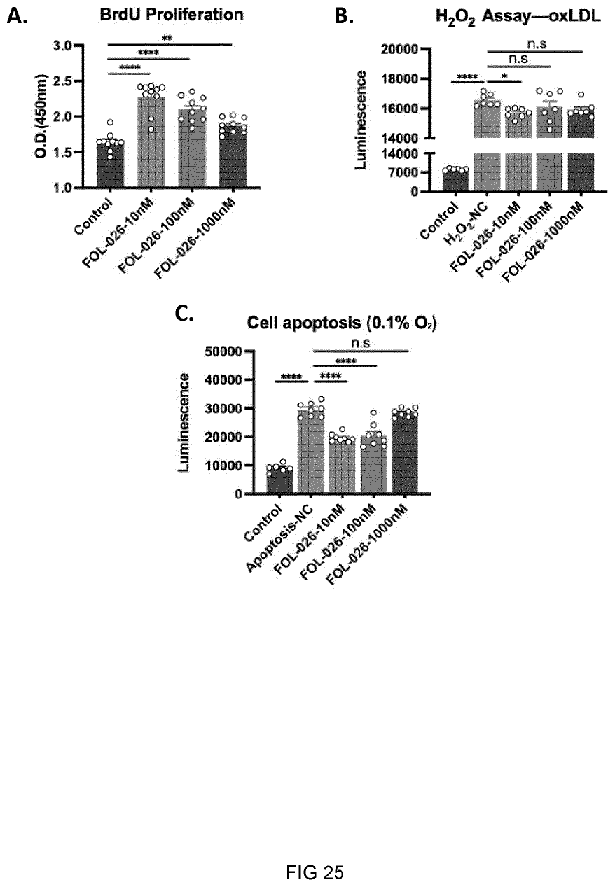

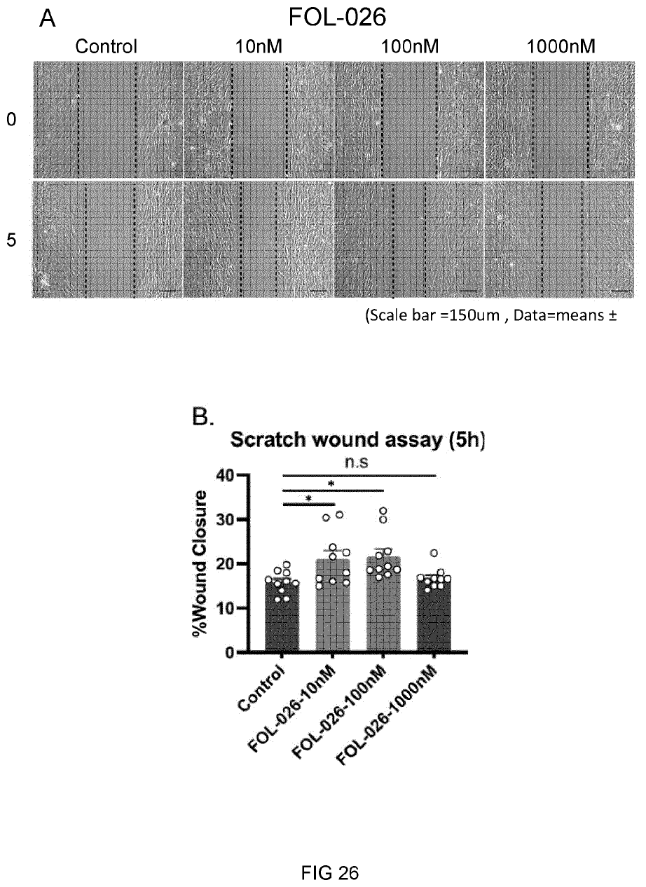

The effect of FOL-026 peptide treatment on smooth muscle cell proliferation (FIG. 25a) and apoptosis (FIG. 25c) induced with low-oxygen condition was evaluated with the uptake of BrdU (n=10 per group) and active caspase-3/-7 (n=6-8 per group) separately. H₂O₂ measurement (FIG. 25b) of smooth muscle cells pre-incubated with FOL-26 peptides after 50 ug/ml oxidized Low-density Lipoprotein (oxLDL) stimulation for 2 hours (n=6-7 per group). Representative images of scratch wound assay in vitro are shown (FIG. 26a) and the ability of wound healing (FIG. 26b) in human coronary smooth muscle cells (HCASMCs) treated with FOL-026 peptides was measured by image J software (n=10 per group). Scale bar=150 um. Data are presented as means±SEM and acquired from 3 to 4 independent replicate tests. *P<0.05, **P<0.01, ***P<0.001, ****P<0.0001 by one-way ANOVA and Dunnett's post-hoc test.

### Results and Conclusion

FOL-026 induces smooth muscle cell proliferation, inhibits apoptosis, reduces ROS formation and accelerates wound closure

### Example 11: Effect of NRP-1 Knock-Down on Smooth Muscle Cell Function Induced with FOL-026 Peptide

### Method

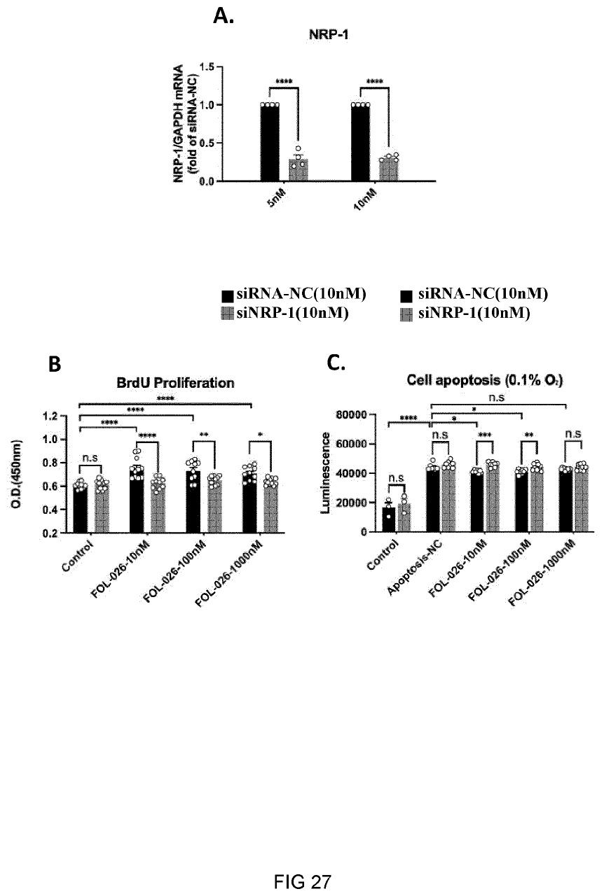

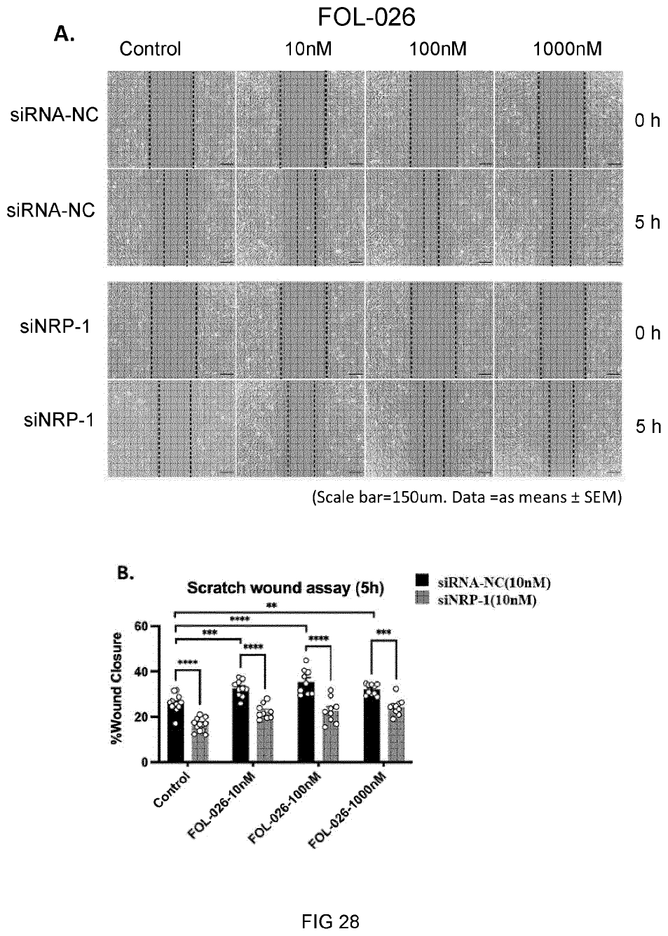

qRT-PCR analysis of NRP-1 mRNA expression in HCASMCs with small interfering RNA transfection for 48 hours (n=4) (FIG. 27a). The assessment of BrdU incorporation (FIG. 27b, n=10-11) and caspase-3/-7 activation (FIG. 27c, n=3-10) in siRNA-NC/siNRP-1-transfected HUVECs treated with indicated concentration of FOL-026 peptide. Representative images of smooth muscle cells at 0 and 5 hours after scratch wounding are shown in FIG. 28a. The wound closure rate (FIG. 28b) of HCASMCs in siRNA-NC and siNRP-1 group stimulated with increasing concentration of FOL-026 peptide was quantified by image J software (n=9-12 per group). Scale bar=150 um. Data are presented as means±SEM and acquired from 3 to 4 independent replicate tests. *P<0.05, **P<0.01, ***P<0.001, ****P<0.0001 by Student's t test for A, two-way ANOVA followed with Sidak's and Dunnett's multiple comparisons test.

### Results and Conclusion

FOL-026 has a positive effect on wound closure in siRNA-NC/siNRP-1-transfected HUVECs.

### Example 12: The FOL-005 Peptide Stimulates Endothelial and Smooth Muscle Cell Proliferation and Wound Healing Ability

### Methods

The endothelial cell proliferation (FIG. 29a) was assessed with BrdU uptake in human umbilical vascular endothelial cells (HUVECs) stimulated with increasing concentration of FOL-005 peptide for 48 hours (n=7-10 per group).

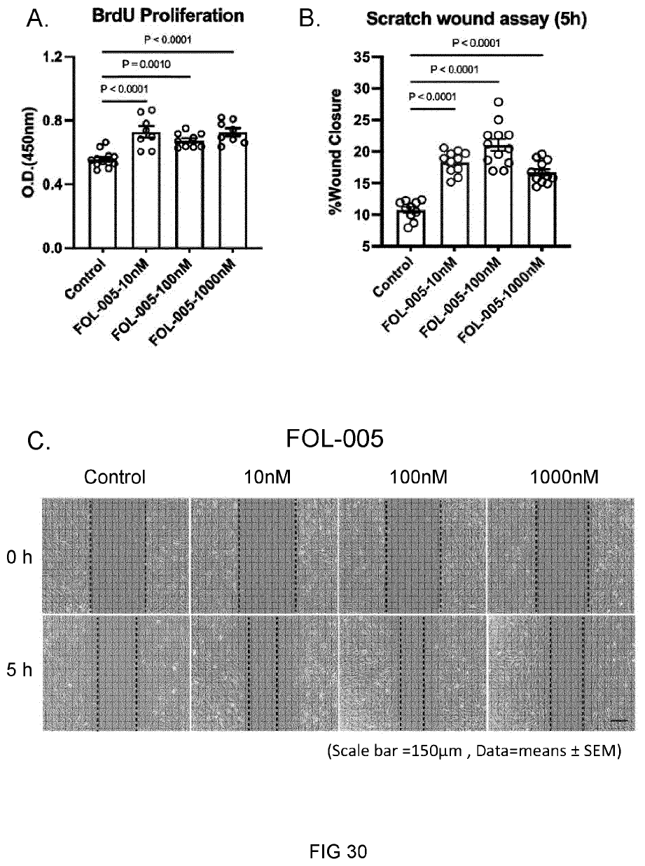

The wound closure rate (FIG. 29b) in HUVECs with indicated dose of FOL-005 treatment was quantified by image J software (n=12-14 per group) and representative images of at 0 and 6 hours after scratch wounding were displayed (FIG. 29c). The effect of FOL-005 peptide on smooth muscle cell proliferation (FIG. 30a) was determined with BrdU incorporation in peptide-treated human coronary smooth muscle cells (HCASMCs). Scratch wound assay (FIG. 30b) of HCASMCs incubated with FOL-005 peptides for 5 hours was measured by image J software (n=10-12 per group) and representative pictures of smooth muscle cell migration into an in vitro scratch injury were shown (FIG. 30c). Scale bar=150 um. Data are presented as means±SEM and acquired from 3 to 4 independent replicate tests. p values were displayed as figure and calculated with One-way ANOVA and Dunnett's post-hoc test.

### Results

The results shown in FIG. 29a indicates that the proliferation of human umbilical vascular endothelial cells (HUVECs) increases with the addition of the FOL-005 peptide. The scratch wound assay in FIGS. 29b and 29c indicates that the FOL-005 peptide accelerate HUVEC wound closure.

The results shown in FIG. 30a indicates that the proliferation of human coronary smooth muscle cells (HCASMCs) increases with the addition of the FOL-005 peptide. The scratch wound assay in FIGS. 30b and 30c indicates that the FOL-005 peptide accelerate HCASMC wound closure.

### Conclusion

The FOL-005 peptide has a positive effect on endothelial and smooth muscle cell proliferation and wound healing ability.

### Example 13: The Effect of FOL-005 Peptide on Tube Formation

### Methods

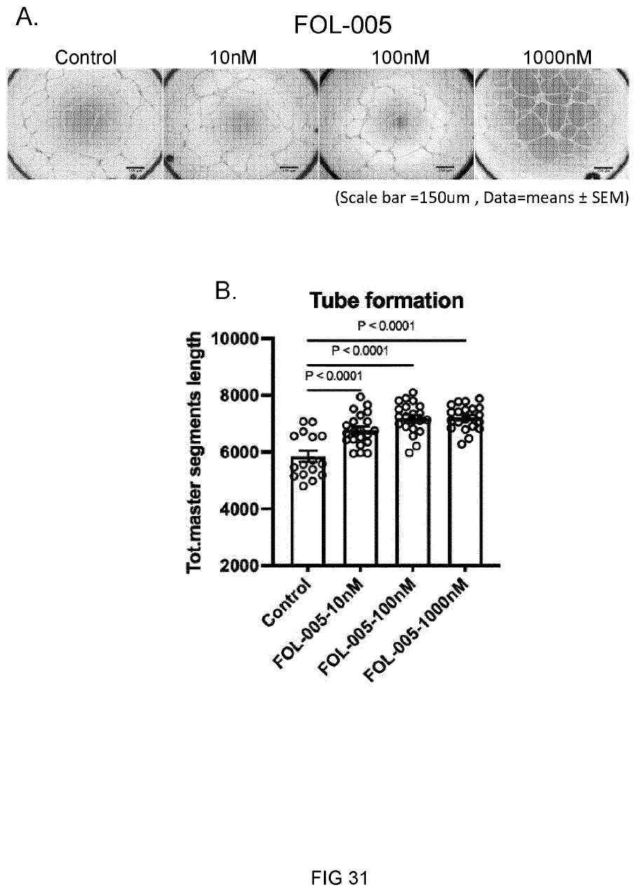

Tube formation in human umbilical vascular endothelial cells (HUVECs) treated with FOL-005 peptides are shown in FIG. 31a. Quantified measurement of total master segments length FIG. 31b was used to evaluate angiogenesis in vitro (n=7 per group). Scale bar=150 um. Data are presented as means±SEM and acquired from 3 to 4 independent replicate tests. p values were displayed as figure and calculated with One-way ANOVA and Dunnett's post-hoc test in B, two-way ANOVA followed with Sidak's and Dunnett's multiple comparisons test.

### Results

The results shown in FIGS. 31a and 31b indicates that addition of the FOL-005 peptide increases the tube formation in human umbilical vascular endothelial cells (HUVECs) Conclusion

FOL-005 peptide addition has a stimulatory effect on endothelial tube formation.

### Sequence Overview

| SEQ ID | | |
|---|---|---|
| NO | Sequence | Notes |
| 1 | X₅X₆SX₇X8YGLR | X₅ is D or G; |
| | | X₆ is I or G; |
| | | X₇ is V or L; |
| | | X₈ is V or A |
| 2 | VDX₂X₃X₄GX₅X₆SX₇X₈YGLR | X₂ is T or V; |
| | | X₃ is Y or P; |
| | | X₄ is D or N; |
| | | X₅ is D or G; |
| | | X₆ is I or G; |
| | | X₇ is V or L; |
| | | X₈ is V or A |
| 3 | VDTYX₄GX₅X₆SX₇X₈YGLR | X₄ is D or N; |
| | | X₅ is D or G; |
| | | X₆ is I or G; |
| | | X₇ is V or L; |
| | | X₈ is V or A |
| 4 | VDTYDGX₅X₆SX₇X₈YGLR | X₅ is D or G; |
| | | X₆ is I or G; |
| | | X₇ is V or L; |
| | | X₈ is V or A |
| 5 | VDTYDGX₅X₆SVVYGLR | X₅ is D or G; |
| | | X₆ is I or G; |
| 6 | VDTYDGGISVVYGLR | FOL-026 |
| 7 | VDTYDGDISVVYGLR | FOL-005 |
| 8 | DTYDGDISVVYGLR | FOL-061 |
| 9 | TYDGDISVVYGLRS | |
| 10 | TYDGDISVVYGLR | |
| 11 | YDGDISVVYGLRS | |
| 12 | YDGDISVVYGLR | |
| 13 | DGDISVVYGLRS | |
| 14 | DGDISVVYGLR | FOL-062 |
| 15 | GDISVVYGLRS | |
| 16 | GDISVVYGLR | FOL-009h |
| 17 | DISVVYGLRS | |
| 18 | DISVVYGLR | |
| 19 | VDVPNGDISLAYGLR | FOL-004 |
| 20 | DVPNGDISLAYGLRS | |
| 21 | DVPNGDISLAYGLR | FOL-007 |
| 22 | VPNGDISLAYGLRS | |
| 23 | VPNGDISLAYGLR | |
| 24 | PNGDISLAYGLRS | |
| 25 | PNGDISLAYGLR | FOL-008 |
| 26 | NGDISLAYGLRS | |
| 27 | NGDISLAYGLR | |
| 28 | GDISLAYGLRS | |
| 29 | GDISLAYGLR | FOL-009 |
| 30 | DISLAYGLRS | |
| 31 | DISLAYGLR | |
| 32 | VDTYDGDGSVVYGLR | FOL-027 |
| 33 | VDVPEGDISLAYGLR | FOL-010 |
| 34 | VDTYDGDISVVYGL | FOL-025 |
| 35 | VDTYDGDISVVYG | FOL-024 |
| 36 | VDTYDGDISVVY | |
| 37 | VDTYDGDISVV | |
| 38 | VDTYDGDISV | |
| 39 | VDTYDGDIS | FOL-019h |
| 40 | VDTYDGRGDSVVYGLR | FOL-002 |
| 41 | VDVPNGDISLAYGL | FOL-016 |
| 42 | VDVPNGDISLAYG | FOL-017 |
| 43 | VDVPNGDISLA | FOL-018 |
| 44 | VDVPNGDIS | FOL-019 |
| 45 | GDPNDGRGDSVVYGLR | FOL-003 |
| 46 | GDPNGDISVVYGLR | FOL-006 |
| 47 | VDVPNGDISLAYRLR | FOL-011 |
| 48 | VDVPEGDISLAYRLR | FOL-012 |
| 49 | VX₁TYDGDISVVYGLR | X₁ is beta D |
| | | FOL-005 (2betaAsp) |
| 50 | VDTYX₄GDISVVYGLR | X₄ is beta D |
| | | FOL-005 (5betaAsp) |
| 51 | VDTYDGX₅ISVVYGLR | X₅ is beta D |
| | | FOL-005 (7betaAsp) |
| 52 | VDTYDGDISVVYGLS | |
| 53 | DTYDGDISVVYGL | |
| 54 | DTYDGDISVVYG | |
| 55 | TYDGDISVVYGL | |
| 56 | DTYDGDISVVY | |
| 57 | TYDGDISVVYG | |
| 58 | YDGDISVVYGL | |
| 59 | DTYDGDISVV | |
| 60 | TYDGDISVVY | |
| 61 | YDGDISVVYG | |
| 62 | DGDISVVYGL | |
| 63 | VDTYDGDIS | |
| 64 | DTYDGDISV | |
| 65 | TYDGDISVV | |
| 66 | YDGDISVVY | |
| 67 | DGDISVVYG | |
| 68 | GDISVVYGL | |
| 69 | ISVVYGLRS | |
| 70 | VDTYDGDI | |
| 71 | DTYDGDIS | |
| 72 | TYDGDISV | |
| 73 | YDGDISVV | |
| 74 | DGDISVVY | |
| 75 | GDISVVYG | |
| 76 | DISVVYGL | |
| 77 | ISVVYGLR | |
| 78 | DVPNGDISLAYGL | |
| 79 | VDVPNGDISLAY | |
| 80 | DVPNGDISLAYG | |
| 81 | VPNGDISLAYGL | |
| 82 | DVPNGDISLAY | |
| 83 | VPNGDISLAYG | |
| 84 | PNGDISLAYGL | |
| 85 | VDVPNGDISL | |
| 86 | DVPNGDISLA | |
| 87 | VPNGDISLAY | |
| 88 | PNGDISLAYG | |
| 89 | NGDISLAYGL | |
| 90 | DVPNGDISL | |
| 91 | VPNGDISLA | |
| 92 | PNGDISLAY | |
| 93 | NGDISLAYG | |
| 94 | GDISLAYGL | |
| 95 | ISLAYGLRS | |
| 96 | VDVPNGDI | |
| 97 | DVPNGDIS | |
| 98 | VPNGDISL | |
| 99 | PNGDISLA | |
| 100 | NGDISLAY | |
| 101 | GDISLAYG | |
| 102 | DISLAYGL | |
| 103 | ISLAYGLR | |
| 104 | VDVPNGRGDSLAYGLR | FOL-001 |
| 105 | X₁₄LX₁₅YGIK | X₁₄ is E or G; |
| | | X₁₅ is S or T |
| 106 | KX₉LAX₁₀X₁₁X₁₂X₁₃IX₁₄LX₁₅YGIK | X₉ is C, P or G; |
| | | X₁₀ is E or G; |
| | | X₁₁ is C, D or l; |
| | | X₁₂ is D, I, S or G; |
| | | X₁₃ is S, D or G; |
| | | X₁₄ is E or G; |
| | | X₁₅ is S or T |
| 107 | KX₉LAX₁₀X₁₁X₁₂X₁₃IX₁₄LSYGIK | X₉ is C, P or G; |
| | | X₁₀ is E or G; |
| | | X₁₁ is C, I or absent; |
| | | X₁₂ is D, G or |
| | | absent; |
| | | X₁₃ is S, G or |
| | | absent; |
| | | X₁₄ is E or G. |
| 108 | KX₉LAX₁₀IX₁₄LSYGIK | X₉ is C, P or G; |
| | | X₁₀ is E or G; |
| | | X₁₄ is E or G. |
| 109 | IELSYGIK | |
| 110 | AEIDSIELSYGIK | FOL-056 |
| 111 | KPLAEIDSIELSYGIK | FOL-014 |
| 112 | KCLAECDSIELSYGIK (Cyclic) | FOL-032 |
| 113 | KPLAEDISIELSYGIK | FOL-037 |
| 114 | KPLAEISDIELSYGIK | FOL-038 |
| 115 | KPLAEIGDIELSYGIK | FOL-039 |
| 116 | KPLAEGDIELSYGIK | FOL-040 |
| 117 | KPLAEIELSYGIK | FOL-041 |
| 118 | KPLAEIDSIELTYGIK | FOL-042 |
| 119 | KPLAEIDGIELSYGIK | FOL-043 |
| 120 | KPLAEIDGIELTYGIK | FOL-044 |
| 121 | KPLAEIGSIELSYGIK | FOL-045 |
| 122 | KGLAEIDSIELSYGIK | FOL-046 |
| 123 | KPLAGIDSIGLSYGIK | FOL-047 |
| 124 | Cyclic KCLAEIDSCELSYGIK | FOL-034 |
| 125 | LAEIDSIELSYGIK | |
| 126 | EIDSIELSYGIK | FOL-057 |
| 127 | IDSIELSYGIK | FOL-058 |
| 128 | DSIELSYGIK | FOL-059 |
| 129 | SIELSYGIK | FOL-060 |
| 130 | CLAEIDSC (Cyclic) | FOL-033 |
| 131 | CFKPLAEIDSIECSYGIK (Cyclic) | FOL-036 |
| 132 | Cyclic CFKPLAEIDSIEC | FOL-035 |
| 133 | KPLAEIDSIELSYGI | |
| 134 | KPLAEIDSIELSYG | |
| 135 | KPLAEIDSIELSY | |
| 136 | KPLAEIDSIELS | |
| 137 | KPLAEIDSIEL | |
| 138 | KPLAEIDSIE | |

### REFERENCES

Gottrup F., A specialized wound-healing center concept: importance of a multidisciplinary department structure and surgical treatment facilities in the treatment of chronic wounds. The American Journal of Surgery. 2004; 187(5):S38-S43.
Kirker K. R., James G. A., In vitro studies evaluating the effects of biofilms on wound-healing cells: a review. APMIS. 2017; 125(4):344-52.
Frykberg R. G., Banks J., Challenges in the Treatment of Chronic Wounds. Adv Wound Care (New Rochelle). 2015; 4(9):560-82.

1. An agent comprising:
a) a peptide selected from the group consisting of:
(i) a peptide comprising or consisting of an amino acid sequence of the general formula:
(SEQ ID NO: 1)
X₅X₆SX₇X₈YGLR
wherein:
X₅ is D or G;
X₆ is I or G;
X₇ is V or L;
X₈ is V or A;
(ii) a peptide comprising or consisting of an amino acid sequence of the general formula:
(SEQ ID NO: 105)
X₁₄LX₁₅YGIK
wherein:
X₁₄ is E or G;
X₁₅ is S or T;
(iii) a peptide comprising or consisting of an amino acid sequence selected from the group consisting of:
(SEQ ID NO: 34)
VDTYDGDISVVYGL,
(SEQ ID NO: 35)
VDTYDGDISVVYG,
(SEQ ID NO: 36)
VDTYDGDISVVY,
(SEQ ID NO: 37)
VDTYDGDISVV,
(SEQ ID NO: 38)
VDTYDGDISV,
(SEQ ID NO: 39)
VDTYDGDIS,
(SEQ ID NO: 40)
VDTYDGRGDSVVYGLR,
(SEQ ID NO: 41)
VDVPNGDISLAYGL,
(SEQ ID NO: 42)
VDVPNGDISLAYG,
(SEQ ID NO: 43)
VDVPNGDISLA,
(SEQ ID NO: 44)
VDVPNGDIS,
(SEQ ID NO: 45)
GDPNDGRGDSVVYGLR,
(SEQ ID NO: 46)
LDGLVRAYDNISPVG,
(SEQ ID NO: 47)
GDPNGDISVVYGLR
(SEQ ID NO: 48)
VDVPNGDISLAYRLR,
(SEQ ID NO: 49)
VDVPEGDISLAYRLR,
(SEQ ID NO: 50)
V(beta-D)TYDGDISVVYGLR,
(SEQ ID NO: 51)
VDTY(beta-D)GDISVVYGLR,
(SEQ ID NO: 52)
VDTYDG(beta-D)ISVVYGLR;
(SEQ ID NO: 130)
CLAEIDSC (Cyclic),
(SEQ ID NO: 131)
CFKPLAEIDSIECSYGIK (Cyclic),
(SEQ ID NO: 132)
Cyclic CFKPLAEIDSIEC,
(SEQ ID NO: 133)
KPLAEIDSIELSYGI,
(SEQ ID NO: 134)
KPLAEIDSIELSYG,
(SEQ ID NO: 135)
KPLAEIDSIELSY,
(SEQ ID NO: 136)
KPLAEIDSIELS,
(SEQ ID NO: 137)
KPLAEIDSIEL,
and
(SEQ ID NO: 138)
KPLAEIDSIE;
b) a polynucleotide encoding upon expression, the peptide of a);
c) a vector comprising the polynucleotide of b); or
d) a cell comprising the polynucleotide of b), or the vector of c),
for use in the treatment of and/or the prevention of a disease or disorder associated with reduced or impaired angiogenesis in a subject.

2. The agent for use according to claim 1, wherein the peptide comprises an amino acid sequence of the general formula:
(SEQ ID NO: 2)
VDX₂X₃X₄GX₅X₆SX₇X₈YGLR
wherein:
X₂ is T or V;
X₃ is Y or P;
X₄ is D or N;
X₅ is D or G;
X₆ is I or G;
X₇ is V or L; and
X₈ is V or A.

3. The agent for use according to any one of the preceding claims, wherein the peptide comprises an amino acid sequence of the general formula:
(SEQ ID NO: 3)
VDTYX₄GX₅X₆SX₇X₈YGLR
wherein:
X₄ is D or N;
X₅ is D or G;
X₆ is I or G;
X₇ is V or L; and
X₈ is V or A.

4. The agent for use according to any one of the preceding claims, wherein the peptide comprises an amino acid sequence of the general formula:
(SEQ ID NO: 4)
VDTYDGZ₇Z₈SZ₁₀Z₁₁YGLR
wherein:
X₅ is D or G;
X₅ is I or G;
X₇ is V or L; and
X₈ is V or A.

5. The agent for use according to any one of the preceding claims, wherein the peptide comprises an amino acid sequence of the general formula:
(SEQ ID NO: 5)
VDTYDGZ₇Z₈SVVYGLR
wherein:
X₅ is D or G; and
X₆ is I or G;

6. The agent for use according to claim 1, wherein the peptide comprises an amino acid sequence of the general formula:
(SEQ ID NO: 106)
KX₉LAX₁₀X₁₁X₁₂X₁₃IX₁₄LX₁₅YGIK
wherein:
X₉ is C, P or G;
X₁₀ is E or G;
X₁₁ is C, D or I;
X₁₂ is D, I, S or G;
X₁₃ is S, D or G;
X₁₄ is E or G; and
X₁, is S or T.

7. The agent for use according to claim 1, wherein the peptide comprises an amino acid sequence of the general formula:
(SEQ ID NO: 107)
KX₉LAX₁₀X₁₁X₁₂X₁₃IX₁₄LSYGIK
wherein:
X₉ is C, P or G;
X₁₀ is E or G;
X₁₁ is C, I or absent;
X₁₂ is D, G or absent;
X₁₃ is S, G or absent; and
X₁₄ is E or G.

8. The agent for use according to claim 1, wherein the peptide comprises an amino acid sequence of the general formula:
(SEQ ID NO: 108)
KX₉LAX₁₀IX₁₄LSYGIK
wherein:
X₉ is C, P or G;
X₁₀ is E or G; and
X₁₄ is E or G.

9. The agent for use according to claim 1, wherein the peptide comprises the amino acid sequence IELSYGIK (SEQ ID NO: 109).

10. The agent for use according to claim 1, with the proviso that if X₁₄ is T, the peptide comprises no more than 25 amino acid residues.

11. The agent for use according to claim 1, wherein the peptide comprises or consists of an amino acid sequence selected from the group consisting of:
(SEQ ID NO: 6)
VDTYDGGISVVYGLR,
(SEQ ID NO: 7)
VDTYDGDISVVYGLR,
(SEQ ID NO: 8)
DTYDGDISVVYGLR,
(SEQ ID NO: 9)
TYDGDISVVYGLRS,
(SEQ ID NO: 10)
TYDGDISVVYGLR,
(SEQ ID NO: 11)
YDGDISVVYGLRS,
(SEQ ID NO: 12)
YDGDISVVYGLR,
(SEQ ID NO: 13)
DGDISVVYGLRS,
(SEQ ID NO: 14)
DGDISVVYGLR,
(SEQ ID NO: 15)
GDISVVYGLRS,
(SEQ ID NO: 16)
GDISVVYGLR,
(SEQ ID NO: 17)
DISVVYGLRS,
(SEQ ID NO: 18)
DISVVYGLR,
(SEQ ID NO: 19)
VDVPNGDISLAYGLR,
(SEQ ID NO: 20)
DVPNGDISLAYGLRS,
(SEQ ID NO: 21)
DVPNGDISLAYGLR,
(SEQ ID NO: 22)
VPNGDISLAYGLRS,
(SEQ ID NO: 23)
VPNGDISLAYGLR,
(SEQ ID NO: 24)
PNGDISLAYGLRS,
(SEQ ID NO: 25)
PNGDISLAYGLR,
(SEQ ID NO: 26)
NGDISLAYGLRS,
(SEQ ID NO: 27)
NGDISLAYGLR,
(SEQ ID NO: 28)
GDISLAYGLRS,
(SEQ ID NO: 29)
GDISLAYGLR,
(SEQ ID NO: 30)
DISLAYGLRS,
(SEQ ID NO: 31)
DISLAYGLR,
(SEQ ID NO: 32)
VDTYDGDGSVVYGLR,
(SEQ ID NO: 33)
VDVPEGDISLAYGLR.
(SEQ ID NO: 110)
AEIDSIELSYGIK,
(SEQ ID NO: 111)
KPLAEIDSIELSYGIK,
(SEQ ID NO: 112)
KCLAECDSIELSYGIK (Cyclic),
(SEQ ID NO: 113)
KPLAEDISIELSYGIK,
(SEQ ID NO: 114)
KPLAEISDIELSYGIK,
(SEQ ID NO: 115)
KPLAEIGDIELSYGIK,
(SEQ ID NO: 116)
KPLAEGDIELSYGIK,
(SEQ ID NO: 117)
KPLAEIELSYGIK,
(SEQ ID NO: 118)
KPLAEIDSIELTYGIK,
(SEQ ID NO: 119)
KPLAEIDGIELSYGIK,
(SEQ ID NO: 120)
KPLAEIDGIELTYGIK,
(SEQ ID NO: 121)
KPLAEIGSIELSYGIK,
(SEQ ID NO: 122)
KGLAEIDSIELSYGIK,
(SEQ ID NO: 123)
KPLAGIDSIGLSYGIK,
(SEQ ID NO: 124)
Cyclic KCLAEIDSCELSYGIK,
(SEQ ID NO: 125)
LAEIDSIELSYGIK,
(SEQ ID NO: 126)
EIDSIELSYGIK,
(SEQ ID NO: 127)
IDSIELSYGIK,
(SEQ ID NO: 128)
DSIELSYGIK,
(SEQ ID NO: 129)
SIELSYGIK,
and
(SEQ ID NO: 109)
IELSYGIK.

12. The agent for use according to claim 1, wherein the peptide comprises or consists of an amino acid sequence VDTYDGGISVVYGLR (SEQ ID NO: 6).

13. The agent for use according to claim 1, wherein the peptide comprises or consists of an amino acid sequence VDTYDGDISVVYGLR (SEQ ID NO: 7).

14. The agent for use according to claim 1, wherein the peptide comprises or consists of an amino acid sequence AEIDSIELSYGIK (SEQ ID NO: 110).

15. The agent for use according to any one of the preceding claims, wherein the peptide comprises no more than 85, such as no more than 80, such as no more than 75, such as no more than 70, such as no more than 65, such as no more than 60, such as nor more than 55, such as no more than 50, such as no more than 55, such as no more than 40 amino acids, such as no more than 35, such as no more than 30, such as no more than 28, such as no more than 26, such as no more than 24, such as no more than 22, such as no more than 20, such as no more than 19, such as no more than 18, such as no more than 17, such as no more than 16, such as no more than 15, such as no more than 14, such as no more than 13, such as no more than 12, such as no more than 11, such as no more than 10 amino acids.

16. The agent for use according to any one of the preceding claims, wherein the peptide comprises at least 2 additional amino acids, such as at least 3, such as at least 4, such as at least 5, such as at least 6, such as at least 7, such as at least 8, such as at least 9, such as at least 10, such as at least 15 or such as at least 20 amino acids conjugated to the N- or C-terminus of the peptide.

17. The agent for use according to any one of the preceding claims, wherein the agent is non-naturally occurring.

18. The agent for use according to any one of the preceding claims, wherein the agent is conjugated to a moiety.

19. The agent for use according to claim 15, wherein the moiety is selected from the group consisting of polyethylene glycol (PEG), monosaccharides, fluorophores, chromophores, radioactive compounds, and cell-penetrating peptides.

20. The agent for use according to any one of the preceding claims, wherein the agent is further modified such as by glycosylation, PEGylation, amidation, esterification, acylation, acetylation and/or alkylation.

21. The agent for use according to any one of the preceding claims, wherein the agent comprises or consists of a tandem repeat comprising two or more repeat units.

22. The agent for use according to claim 21, wherein the repeat unit comprises or consists of the amino acid sequence of any one or more of the sequences as described in the preceding claims.

23. The agent for use according to any of the preceding claims, wherein the agent is fused to another polypeptide.

24. The agent for use according to claim 23, wherein the said polypeptide is selected from the group consisting of glutathione-S-transferase (GST) and protein A.

25. The agent for use according to any of the preceding claims, wherein the agent is fused to a tag.

26. The agent for use according to claim 25, wherein the tag is an oligo-histidine tag.

27. The agent for use according to any of the preceding claims, wherein the agent is cyclic.

28. The agent for use according to any of the preceding claims, wherein the agent is capable of forming at least one intramolecular cysteine bridge.

29. The agent for use according to any one of the preceding claims, wherein the agent is a variant of the peptide, wherein the variant comprises or consists of a sequence wherein any one amino acid has been altered for another proteinogenic or non-proteinogenic amino acid, with the proviso that no more than five amino acids are so altered.

30. The agent for use according to claim 29, wherein the variant comprises or consists of a sequence wherein no more than five amino acids are altered for another proteinogenic or non-proteinogenic amino acid, such as no more than 4 amino acids, such as no more than 3 amino acids, such as no more than 2 amino acids, such as no more than 1 amino acid is altered.

31. The agent for use according to any one of the preceding claims, wherein one or more amino acids are conservatively substituted.

32. The agent for use according to any one of the preceding claims, wherein the peptide comprises or consists of one or more additional amino acids, inserted at the N- and/or C-terminus and/or internally within the sequence.

33. The agent for use according to any one of the preceding claims, wherein the peptide has one additional amino acid.

34. The agent for use according to any of the preceding claims, wherein the agent further comprises a detectable moiety.

35. The agent for use according to any one of the preceding claims, wherein the agent is in a composition.

36. The agent for use according to claim 35, wherein the composition is a pharmaceutical composition.

37. The agent for use according to claim 35, wherein the composition is a cosmetic composition.

38. The agent for use according to any one of the preceding claims, wherein the composition is a coating.

39. The agent for use according to any one of claims 35 to 38, wherein the composition is a coating of an implant.

40. The agent for use according to claim 39, wherein the implant is of a biomaterial.

41. The agent for use according to claim 40, wherein the biomaterial is bone.

42. The agent for use according to claim 39, wherein the implant is a medical device.

43. The agent for use according to claim 42, wherein the medical device is a stent.

44. The agent for use according to any one of the preceding claims, wherein the agent increases angiogenesis in the subject.

45. The agent for use according to any one of the preceding claims, wherein the agent is an angiogenesis inducer.

46. The agent for use according to any one of the preceding claims, wherein the agent is capable of improving myocyte survival in cardiovascular disease.

47. The agent for use according to any one of the preceding claims, wherein the agent is capable of improving neural cell survival in cerebrovascular disease.

48. The agent for use according to any one of the preceding claims, wherein the disease or disorder is selected from the group consisting of:
i. a disease of the circulatory system,
ii. an injury of external cause,
iii. a disease of the immune system,
iv. a disease of the nervous system, and
v. a disease of the musculoskeletal system or connective tissue.

49. The agent for use according to claim 48, wherein the disease of the circulatory system is selected from the group consisting of:
i. an ischaemic heart disease,
ii. a cerebrovascular disease,
iii. a disease of coronary artery,
iv. a disease of the arteries, arterioles, or capillaries,
v. a symptom, sign, or clinical finding of the circulatory system,
vi. a disease of the myocardium or cardiac chambers, and
vii. an endocrine, nutritional or metabolic disease.

50. The agent for use according to claim 49, wherein the ischaemic heart disease is selected from the group consisting of:
i. acute ischaemic heart disease, such as myocardial infarction, such as acute myocardial infarction and unspecified acute ischaemic heart disease (acute coronary syndrome),
ii. chronic ischaemic heart disease, such as other specific chronic ischaemic heart disease (cardiovascular arteriosclerosis),
iii. angina pectoris, such as unstable angina pectoris, and
iv. obstructive arteriosclerosis.

51. The agent for use according to claim 50, wherein the myocardial infarction is ST elevation myocardial infarction (STEMI) or non-ST elevation myocardial infarction (NSTEMI).

52. The agent for use according to claim 51, wherein the STEMI or NSTEMI presents with subsequent certain current complications, such as within a 28 day period.

53. The agent for use according to claim 48, wherein the disease of the circulatory system is cardiovascular sclerosis.

54. The agent for use according to claim 48, wherein the disease of the circulatory system is systemic sclerosis or associated with systemic sclerosis.

55. The agent for use according to claim 49, wherein the cerebrovascular disease is selected from the group consisting of:
i. cerebral ischaemia, such as cerebral ischaemic stroke (stroke), such as cerebral infarction, and
ii. asymptomatic stenosis of intracranial or extracranial artery (cerebral arteriosclerosis).

56. The agent for use according to claim 55, wherein the cerebral ischaemic stroke is associated with a patient history of transient ischaemic attack (TIA) or cerebral infarction without residual deficits.

57. The agent for use according to any one of claims 55 or 56, wherein the cerebral ischaemic stroke is associated with a patient history of traumatic brain injury or sequelae of cerebrovascular disease.

58. The agent for use according to claim 49, wherein the cerebrovascular disease is selected from the group consisting of:
i. nontraumatic subarachnoid haemorrhage,
ii. nontraumatic intracerebral haemorrhage,
iii. other and unspecified nontraumatic intracranial haemorrhage,
iv. cerebral infarction,
v. occlusion and stenosis of precerebral arteries not resulting in cerebral infarction,
vi. occlusion and stenosis of cerebral arteries, not resulting in cerebral infarction,
vii. other cerebrovascular diseases,
viii. cerebrovascular disorder associated with other diseases (cerebrovascular disorders classified elsewhere), and
ix. sequelae of cerebrovascular disease.

59. The agent for use according to claim 49, wherein the disease of the arteries, arterioles, or capillaries is selected from the group consisting of:
i. atherosclerosis,
ii. aortic aneurysm and dissection,
iii. an other aneurysm,
iv. an other peripheral vascular disease,
v. arterial embolism and thrombosis,
vi. atheroembolism,
vii. septic arterial embolism,
viii. an other disorder of arteries and arterioles,
ix. a disease of the capillaries, and
x. a disorder of the arteries, arterioles, or capillaries associated with another disease (a disorder of the arteries, arterioles, or capillaries in a disease classified elsewhere).

60. The agent for use according to claim 49, wherein the disease of the arteries, arterioles, or capillaries is chronic arterial occlusive disease, such as atherosclerotic chronic arterial occlusive disease or vascular sclerosis.

61. The agent for use according to claim 49, wherein the symptom, sign, or clinical finding of the circulatory system is a symptom or sign involving the circulatory system, such as an abnormal blood-pressure reading without diagnosis, such as cardiac arrest.

62. The agent for use according to claim 49, wherein the disease of the myocardium or cardiac chambers is cardiomyopathy, such as dilated cardiomyopathy.

63. The agent for use according to claim 48, wherein the disease of the immune system is non-organ specific systemic autoimmune disorder, such as vasculitis, such as thromboangiitis obliterans (Buerger's disease).

64. The agent for use according to claim 48, wherein the disease of the nervous system is selected from the group consisting of:
i. a movement disorder, such as parkinsonism, such as Parkinson's disease,
ii. multiple sclerosis or other white matter disorders, such as multiple sclerosis, and
iii. disorders with neurocognitive impairment as a major feature, such as Alzheimer's disease.

65. The agent for use according to claim 48, wherein the disease of the musculoskeletal system or connective tissue is a condition associated with the spine, such as herniated disc.

66. The agent for use according to claim 48, wherein the disease of the circulatory system, the disease of the immune system, the disease of the nervous system, or the disease of the musculoskeletal system or connective tissue is or is associated with diabetes mellitus.

67. The agent for use according to claim 66, wherein the diabetes mellitus is selected from Type 1 diabetes mellitus and Type 2 diabetes mellitus.

68. The agent for use according to any one of the preceding claims, wherein the subject is suffering from diabetes mellitus.

69. The agent for use according to any one of the preceding claims, wherein the subject is a mammal.

70. The agent for use according to claim 69, wherein the mammal is a human.

71. A method for treating or preventing a disease or disorder in a subject, wherein the disease or disorder is selected from the group consisting of:
i. a disease of the circulatory system,
ii. an injury of external cause,
iii. a disease of the immune system,
iv. a disease of the nervous system, and
v. a disease of the musculoskeletal system or connective tissue,
the method comprising administering a therapeutically effective amount of an agent as defined in any one of claims 1 to 47 to an subject in need thereof.

72. Use of an agent as defined in any one of claims 1 to 47 for the manufacture of a medicament for the treatment or prevention of a disease or disorder selected from the group consisting of:
i. a disease of the circulatory system,
ii. an injury of external cause,
iii. a disease of the immune system,
iv. a disease of the nervous system, and
v. a disease of the musculoskeletal system or connective tissue.

73. An agent comprising:
a) a peptide selected from the group consisting of:
(i) a peptide comprising or consisting of an amino acid sequence of the general formula:
(SEQ ID NO: 1)
X₅X₆SX₇X₈YGLR
wherein:
X₅ is D or G;
X₈ is I or G;
X₇ is V or L;
X₈ is V or A;
(ii) a peptide comprising or consisting of an amino acid sequence of the general formula:
(SEQ ID NO: 105)
X₁₄LX₁₅YGIK
wherein:
X₁₄ is E or G;
X₁₅ is S or T;
(iii) a peptide comprising or consisting of an amino acid sequence selected from the group consisting of:
(SEQ ID NO: 34)
VDTYDGDISVVYGL,
(SEQ ID NO: 35)
VDTYDGDISVVYG,
(SEQ ID NO: 36)
VDTYDGDISVVY,
(SEQ ID NO: 37)
VDTYDGDISVV,
(SEQ ID NO: 38)
VDTYDGDISV,
(SEQ ID NO: 39)
VDTYDGDIS,
(SEQ ID NO: 40)
VDTYDGRGDSVVYGLR,
(SEQ ID NO: 41)
VDVPNGDISLAYGL,
(SEQ ID NO: 42)
VDVPNGDISLAYG,
(SEQ ID NO: 43)
VDVPNGDISLA,
(SEQ ID NO: 44)
VDVPNGDIS,
(SEQ ID NO: 45)
GDPNDGRGDSVVYGLR,
(SEQ ID NO: 46)
LDGLVRAYDNISPVG,
(SEQ ID NO: 47)
GDPNGDISVVYGLR
(SEQ ID NO: 48)
VDVPNGDISLAYRLR,
(SEQ ID NO: 49)
VDVPEGDISLAYRLR,
(SEQ ID NO: 50)
V(beta-D)TYDGDISVVYGLR,
(SEQ ID NO: 51)
VDTY(beta-D)GDISVVYGLR,
(SEQ ID NO: 52)
VDTYDG(beta-D)ISVVYGLR;
(SEQ ID NO: 130)
CLAEIDSC (Cyclic),
(SEQ ID NO: 131)
CFKPLAEIDSIECSYGIK (Cyclic),
(SEQ ID NO: 132)
Cyclic CFKPLAEIDSIEC,
(SEQ ID NO: 133)
KPLAEIDSIELSYGI,
(SEQ ID NO: 134)
KPLAEIDSIELSYG,
(SEQ ID NO: 135)
KPLAEIDSIELSY,
(SEQ ID NO: 136)
KPLAEIDSIELS,
(SEQ ID NO: 137)
KPLAEIDSIEL,
and
(SEQ ID NO: 138)
KPLAEIDSIE;
b) a polynucleotide encoding upon expression, the peptide of a);
c) a vector comprising the polynucleotide of b); or
d) a cell comprising the polynucleotide of b), or the vector of c)
for use in improving vascularisation post surgery in a subject.

74. The agent for use claim 73, wherein the surgery is surgery of the cardiovascular system.

75. The agent for use according to any one of claims 73 or 74, wherein the surgery of the cardiovascular system is a vascular graft.

76. The agent for use according to any one of claims 73 to 75, wherein the subject has undergone transplant.

77. The agent for use according to claim 76, wherein the transplant is of an organ, of a tissue, or of a cell.

78. The agent for use according to claim 77, wherein the transplant of a cell is of bone marrow cells.

79. The agent for use according to claim 77, wherein the transplant of an organ is of a heart or a cardiovascular tissue.

80. A method for inducing vascularization, said method comprising administering the agent as defined in any one of claims 1 to 47 to a subject.

81. An implant comprising a peptide selected from the group consisting of:
(i) a peptide comprising or consisting of an amino acid sequence of the general formula:
(SEQ ID NO: 1)
X₅X₆SX₇X₈YGLR
wherein:
X₅ is D or G;
X₆ is I or G;
X₇ is V or L;
X₈ is V or A;
(ii) a peptide comprising or consisting of an amino acid sequence of the general formula:
(SEQ ID NO: 105)
X₁₄LX₁₅YGIK
wherein:
X₁₄ is E or G;
X₁₅ is S or T;
(iii) a peptide comprising or consisting of an amino acid sequence selected from the group consisting of:
(SEQ ID NO: 34)
VDTYDGDISVVYGL,
(SEQ ID NO: 35)
VDTYDGDISVVYG,
(SEQ ID NO: 36)
VDTYDGDISVVY,
(SEQ ID NO: 37)
VDTYDGDISVV,
(SEQ ID NO: 38)
VDTYDGDISV,
(SEQ ID NO: 39)
VDTYDGDIS,
(SEQ ID NO: 40)
VDTYDGRGDSVVYGLR,
(SEQ ID NO: 41)
VDVPNGDISLAYGL,
(SEQ ID NO: 42)
VDVPNGDISLAYG,
(SEQ ID NO: 43)
VDVPNGDISLA,
(SEQ ID NO: 44)
VDVPNGDIS,
(SEQ ID NO: 45)
GDPNDGRGDSVVYGLR,
(SEQ ID NO: 46)
LDGLVRAYDNISPVG,
(SEQ ID NO: 47)
GDPNGDISVVYGLR
(SEQ ID NO: 48)
VDVPNGDISLAYRLR,
(SEQ ID NO: 49)
VDVPEGDISLAYRLR,
(SEQ ID NO: 50)
V(beta-D)TYDGDISVVYGLR,
(SEQ ID NO: 51)
VDTY(beta-D)GDISVVYGLR,
(SEQ ID NO: 52)
VDTYDG(beta-D)ISVVYGLR;
(SEQ ID NO: 130)
CLAEIDSC (Cyclic),
(SEQ ID NO: 131)
CFKPLAEIDSIECSYGIK (Cyclic),
(SEQ ID NO: 132)
Cyclic CFKPLAEIDSIEC,
(SEQ ID NO: 133)
KPLAEIDSIELSYGI,
(SEQ ID NO: 134)
KPLAEIDSIELSYG,
(SEQ ID NO: 135)
KPLAEIDSIELSY,
(SEQ ID NO: 136)
KPLAEIDSIELS,
(SEQ ID NO: 137)
KPLAEIDSIEL,
and
(SEQ ID NO: 138)
KPLAEIDSIE.

82. The implant according to claim 81, wherein the implant is coated with a composition comprising an agent according to any one of the preceding claims.

83. The implant according to claim 81, wherein the implant is of a biomaterial.

84. The implant according to claim 83, wherein the biomaterial is bone.

85. The implant according to claim 81, wherein the implant is a medical device.

86. The implant according to claim 85, wherein the medical device is a stent.
